SEQUENCE LISTING (18 sequences)

SEQ ID NO: 1 - DNA, 10 bp, artificial
  promoter region recognition motif for AP-1
  misc_feature (1)..(2): n is a, c, g, or t
  misc_feature (5)..(5): n is a, c, g, or t
  misc_feature (10)..(10): n is a, c, g, or t
nntknstcan

SEQ ID NO: 2 - DNA, 10 bp, artificial
  promoter region recognition motif for ETS
  misc_feature (1)..(1): n is a, c, g, or t
  misc_feature (10)..(10): n is a, c, g, or t
nvmggawryn

SEQ ID NO: 3 - DNA, 12 bp, artificial
  promoter region recognition motif for IRF
  misc_feature (1)..(6): n is a, c, g, or t
  misc_feature (8)..(12): n is a, c, g, or t
nnnnnnvnnnnn

SEQ ID NO: 4 - DNA, 20 bp, artificial
  promoter region recognition motif for CTCF
  misc_feature (1)..(4): n is a, c, g, or t
  misc_feature (13)..(13): n is a, c, g, or t
  misc_feature (18)..(20): n is a, c, g, or t
nnnnydmcmyctnstggnnn

SEQ ID NO: 5 - DNA, 10 bp, artificial
  promoter region recognition motif for ETS
vdbaggwwrb

SEQ ID NO: 6 - DNA, 20 bp, artificial
  promoter region recognition motif for CTCF
  misc_feature (1)..(4): n is a, c, g, or t
  misc_feature (13)..(13): n is a, c, g, or t
  misc_feature (18)..(20): n is a, c, g, or t
nnnnydmcmyctnstggnnn

SEQ ID NO: 7 - DNA, 10 bp, artificial
  promoter region recognition motif for Runx
  misc_feature (1)..(1): n is a, c, g, or t
  misc_feature (10)..(10): n is a, c, g, or t
nhraccrcan

SEQ ID NO: 8 - DNA, 12 bp, artificial
  promoter region recognition motif for Nrf
  misc_feature (1)..(2): n is a, c, g, or t
nnrsgcvbgcgc

SEQ ID NO: 9 - DNA, 10 bp, artificial
  promoter region recognition motif for ETS
  misc_feature (1)..(1): n is a, c, g, or t
  misc_feature (9)..(10): n is a, c, g, or t
nvmggawrnn

SEQ ID NO: 10 - DNA, 10 bp, artificial
  promoter region recognition motif for Runx
  misc_feature (9)..(10): n is a, c, g, or t
hrrccdchnn

SEQ ID NO: 11 - DNA, 20 bp, artificial
  promoter region recognition motif for CTCF
  misc_feature (1)..(4): n is a, c, g, or t
  misc_feature (7)..(7): n is a, c, g, or t
  misc_feature (13)..(13): n is a, c, g, or t
  misc_feature (15)..(15): n is a, c, g, or t
  misc_feature (17)..(20): n is a, c, g, or t
nnnnydncmyctnsngnnnn

SEQ ID NO: 12 - DNA, 10 bp, artificial
  promoter region recognition motif for AP-1
  misc_feature (1)..(2): n is a, c, g, or t
  misc_feature (6)..(6): n is a, c, g, or t
  misc_feature (8)..(8): n is a, c, g, or t
  misc_feature (10)..(10): n is a, c, g, or t
nntkdnwnan

SEQ ID NO: 13 - DNA, 10 bp, artificial
  promoter region recognition motif for AP-1
  misc_feature (1)..(2): n is a, c, g, or t
  misc_feature (10)..(10): n is a, c, g, or t
nntkastman

SEQ ID NO: 14 - DNA, 20 bp, artificial
  promoter region recognition motif for CTCF
  misc_feature (1)..(4): n is a, c, g, or t
  misc_feature (13)..(13): n is a, c, g, or t
  misc_feature (17)..(20): n is a, c, g, or t
nnnnydmcmyctnstgnnnn

SEQ ID NO: 15 - DNA, 10 bp, artificial
  promoter region recognition motif for ETS
  misc_feature (1)..(1): n is a, c, g, or t
  misc_feature (10)..(10): n is a, c, g, or t
nvmggawryn

SEQ ID NO: 16 - DNA, 12 bp, artificial
  promoter region recognition motif for IRF
  misc_feature (1)..(2): n is a, c, g, or t
  misc_feature (7)..(8): n is a, c, g, or t
nnttyynnttyy

SEQ ID NO: 17 - DNA, 18 bp, artificial
  synthetic oligonucleotide primer
acgagaaagtggagatcg

SEQ ID NO: 18 - DNA, 15 bp, artificial
  synthetic oligonucleotide primer
gccttctggaaaaca

(19) United States
(12) Patent Application Publication
(10) Pub. No.: US 2019/0269705 A1
(43) Pub. Date: Sep. 5, 2019

(54) METHODS AND MATERIALS FOR TREATING GRAFT VERSUS HOST DISEASE

(71) Applicants: Regents of the University of Minnesota, Minneapolis, MN (US); Vanderbilt University, Nashville, TN (US)

(72) Inventors: Bruce R. Blazar, Golden Valley, MN (US); Katelyn Paz, Minneapolis, MN (US); Jeffrey Rathmell, Nashville, TN (US); Marc Johnson, Nashville, TN (US)

(21) Appl. No.: 16/201,074

(22) Filed: Nov. 27, 2018

Related U.S. Application Data
(60) Provisional application No. 62/590,898, filed on Nov. 27, 2017.

(51) Int. Cl. A61K 31/655; A61P 37/06

(52) U.S. Cl. CPC A61K 31/655; A61P 37/06

(57) ABSTRACT

This document provides methods and materials for treating or preventing GVHD. For example, methods and materials for using a glutaminolysis inhibitor to treat or prevent GVDH are provided.

### CROSS-REFERENCE TO RELATED APPLICATIONS

This application claims the benefit of U.S. Patent Application Ser. No. 62/590,898, filed on Nov. 27, 2017. The disclosure of the prior application is considered part of (and is incorporated by reference in) the disclosure of this application.

### STATEMENT REGARDING FEDERAL FUNDING

This invention was made with government support under CA142106 awarded by the National Institutes of Health. The government has certain rights in the invention.

### BACKGROUND

### 1. Technical Field

This document relates to methods and materials for treating or preventing graft-versus-host-disease (GVHD). For example, this document provides methods and materials for using an inhibitor of glutaminolysis to treat or prevent GVDH.

### 2. Background Information

Currently, therapies for GVHD are limited, and typically treat the symptoms as opposed to the actual disease. Accordingly, novel therapies for GVHD would be beneficial.

### SUMMARY

This document provides methods and materials for treating or preventing GVHD. For example, this document provides methods and materials for using a glutaminolysis inhibitor to treat or prevent GVDH. In some cases, the methods and materials described herein can reduce morbidity and/or mortality in subjects who undergo allogeneic hematopoietic stem-cell transplantation.

As demonstrated herein, glutaminolysis is required by donor T cells to induce cGVHD, and 6-Diazo-5-Oxo-L-Norleucine (DON) can inhibit glutaminolysis. Having the ability to inhibit glutaminolysis provides a unique and unrealized opportunity to treat or prevent GVDH.

In general, one aspect of this document features a method for treating or preventing GVHD in a subject. The method includes, or consists essentially of, administering a therapeutically effective amount of a glutaminolysis inhibitor to a subject. The glutaminolysis inhibitor can be DON. The DON can be administered to the subject at a dose of about 0.5 mg to about 50 mg of the DON per kilogram (kg) of the subject (e.g., at a dose of about 1.6 mg of the DON per kg of the subject). The glutaminolysis inhibitor can be administered to the subject at least once a day. The glutaminolysis inhibitor can be administered intraperitoneally. The subject can have received a hematopoietic stem cell transplant (e.g., an allogeneic hematopoietic stem-cell transplant or a bone marrow transplant). The administering can occur prior to the subject receiving the hematopoietic stem cell transplant. The administering can occur coincidentally with the subject receiving the hematopoietic stem cell transplant. The administering can occur after the subject has received the hematopoietic stem cell transplant. The GVHD can be treated in the subject when the GVHD or one or more symptoms associated with the GVHD is reversed, alleviated or inhibited. The GVHD can be prevented in the subject when the GVHD or one or more symptoms associated with GVHD is avoided or precluded. The GVHD can be chronic GVHD. The GVHD can be acute GVHD.

In another aspect, this document features a method for treating or preventing GVHD in a subject. The method includes, or consists essentially of, contacting donor T cells with a therapeutically effective amount of a glutaminolysis inhibitor. The glutaminolysis inhibitor can be DON. The donor T cells can be hematopoietic stem cells. The donor T cells can be contacted with the glutaminolysis inhibitor ex vivo.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic diagram showing that the conversion of glutamine to alpha-ketoglutarate is a primary way for carbon to enter the TCA cycle.

FIGS. 2A-D show that activated T cells use glutamine and glutamate, and rely on pyruvate transport. Naïve CD4 cells were isolated from WT mice and A) maintained in IL-7 (N), stimulated on CD3/CD28 (S), or stimulated and given rapamycin for 24 hours (S/R). Metabolites extracted for mass spectrometry and presented as fold change from naïve. B) Oxygen Consumption Rate (OCR) (left) assayed from naïve CD4 cells from WT mice stimulated for 3 days on CD3/CD28, injected with Vehicle, CB839, UK5099, or combination UK5099+ CB839. OCR at timepoint 200 min (right). (C-D) Abundance of metabolites (left) and fractional labeling (right) of stimulated CD4+ T cells in the presence of CB839 and 13C-glucose. Mean +/− Standard Deviation shown from n=3 experiments.

FIGS. 3A-F look at changes in glycolysis metabolite levels. A) Relative expression of glutamine pathway genes, data from ImmGen. B) Ratio of glutamate:glutamine metabolite levels in IL-7 (naïve, N), CD3/CD28 (stimulated, S), or CD3/CD28 plus rapamycin (S/R) in wild type CD4+ T cells. (C-F) Additional intracellular metabolite abundance (left) and fraction labeled from ¹³C-glucose (right) extracted from wild type CD4s treated with vehicle or CB839 for C) Amino acids Serine, alanine, and glycine. D) Glycolytic intermediates G6P, F 16BP. E) Lactate and Pyruvate. F) Nucleotide precursor N-carbamoyl L-aspartate.

FIGS. 4A-E show that Th1 and Th17 cells differ in their use of glutaminolysis. Naïve CD4+ cells were isolated from WT mice and A) maintained in IL-7 (N), differentiated into Th1 (1), Th2 (2), Th17 (17), or Treg (R) and metabolites extracted for mass spectrometry and presented as fold change from naïve. B) Cytokine production from Th1 (top) and Th17 (bottom) differentiated T cells in the presence (left) or absence (right) of glutamine. C) Proliferation of Th1 and Th17 cells with or without glutamine after 4 days of differentiation. D) Metabolite heatmap of wild type T cells skewed into Th1 and Th17 cells in the presence or absence of 500 nM CB839. Red=upregulated, green=downregulated. E) Partial principle component analysis of the metabolites from D.

FIGS. 5A-D examines glutamate and glutamine. A) Ratio of intracellular metabolites glutamate:glutamine from CD4+ T cells in Th1, Th2, Th17, and Treg skewing conditions. B) GLS expression from RNA-Seq from FIG. 2D. C) GLS2 expression from RNA-Seq from FIG. 2D on the same scale as GLS expression (left) and in smaller scale. P values are determined from RNA-Seq analysis. D) Uptake (positive numbers) and secretion (negative numbers) of CB839 treated wild type CD4+ T cells in Th1 and Th17 skewing conditions over 5 days as measured by Nuclear Magnetic Resonance (NMR) (average of 3 experiments).

FIGS. 6A-D show that GLS1 is important for activation of T cells but not maintenance or development. (A-C) Flow cytometry staining of splenocytes isolated from wild type (WT) or GLS1^{FL/FL}+CD4-CRE (GLS KO) presented as percent of total. D) Cell size, proliferation, and surface marker expression of CD4+ T cells isolated from WT or GLS KO mice and stimulated on CD3/CD28 for 3 days. B and D shows means +/− Standard Deviation representative of n=3 experiments.

FIGS. 7A-E examine T cells from GLS^{fl/fl}CD4-Cre mice which lack expression of GLS. (A-C) CD4+ and CD8+ T cells isolated from wild type (WT) and GLS1 knockout (GLS KO) T cells. A) PCR of genomic DNA outside of deletion (exons 9 and 12, left) and inside deletion (Exons 10 and 11, right), from two KO and two WT animals. C) FACS plots of baseline cell size, and markers of activation in naïve GLS KO and WT T cells, representative of 3 experiments. D) Cell size (FSC by flow cytometry) in activated CD4+ T cells from WT and GLS KO animals. E) pS6 expression in 3 day activated CD8+ T cells on CD3/CD28 (left) and average signal from n=3 replicates (right).

FIGS. 8A-I show that effector responses are enhanced in activated T cells deficient for GLS1. (A-F) Cytokine production of naïve CD4+ T cells activated over 5 days, then stimulated with PMA and ionomycin for 4 hours. A) Wild type and GLS KO T cells, B) Average percent total IFNγ + producers (left), percent double positive IFNγ +IL2+ producers (middle), and the median fluorescence intensity (MFI) (right) of IFNγ + cells in A. C) Tbet protein expression in WT (black), GLS KO (red), and isotype control (grey filled). Representative of n=2 experiments. D-F) Same as in A, but wild type CD4+ T cells were activated in the presence of CB839. G) Cytokine production of naïve CD8+ T cells from WT and GLS KO T cells activated on CD3/CD28+ IL2 over 4 days. H) Granzyme B expression in activated CTLs from G (left) and average MFI (right) of GranB+ cells. I) Live cell counts measured by propidium iodide stain.

FIGS. 9A-E examine GLS-deficient T cells. A) Foxp3 expression in CD4+ Th0 skewing conditions (αCD3 and feeder layer of splenocytes) after 5 days in the presence of vehicle or CB839. B) Viability (left) and cell count (right) of GLS KO and WT CD8+ T cells stimulated over 4 days on αCD3/CD28 and IL-2. C) Viability at day 3 (left) and day 5 (right) of CD4+ T cells from wild type C57BL6 mice in Th0 skewing conditions (αCD3 and splenocytes feeder layer) over 5 days in the presence of vehicle or CB839. D) Granzyme B expression of CD8+ T cells stimulated for 4 days with αCD3/CD28 and IL-2, in the presence of vehicle or CB839. E) Extracellular Acidification Rate (ECAR) as a measure of instantaneous T cell activation with αCD3/CD28 injection in the presence of vehicle or CB839.

FIGS. 10A-E examine T cell effector subsets in GLS-deficient T cells. A) DCFDA (total cellular reactive oxygen species) expression at Day 5 by flow cytometry. B) Average MFI, representative of 3 experiments. (C-E) CD4+ cells treated with vehicle or CB839 in Th1, Th17, and Treg skewing conditions C) CD279 (PD-1) expression at Day 5. D) Average MFI at Day 5 of KLRG1, CD279 (PD-1), and CD127 (IL-7 Receptor) (n=3, representative of 3 experiments). E) Foxp3+ expression by flow cytometry in wild type CD4+ T cells in Th17 skewing conditions plus CB839 (green) or vehicle (Black) compared to control IgG (grey filled).

FIGS. 11A-M show that differential cytokine production in GLS1 deficient Th1 and Th17 cells. (A-D) Naïve CD4+ T cells from WT and GLS KO T cells differentiated into Th1, Th17, and Treg cells. A) Cell size at day 3 and day 5 of Th1 KO cells and Th17 KO cells. B) Cell size at day 3 and day 5 of CB839-treated Th1 cells and CB839-treated Th17 cells. C) ³H-2-deoxyglucose uptake in Th1 and Th17 differentiation media after 5 days (left), and Extracellular Acidification Rate (ECAR) of Th1 and Th17 differentiated T cells after 5 days (right). D) Proliferation of Th1 and Th17 cells in WT (black) and GLS KO (red). E) Differentiation of Th1, Th17, and Treg cells in WT (black) and GLS KO (green). (F-M) Cytokine production in GLS KO T cells over 5 days of skewing. F) Cytokine production in WT and GLS KO T cells under Th1 skewing conditions and Th17 skewing conditions. G) Average percent change from WT of cytokine production (n=5 experiments) in Th1 and Th17 over 5 days of skewing. H) Transcription factor expression of Th1, Th17 and Treg in WT cells (black) or GLS KO T cells (red) over 5 days. I) Average percent change from WT of transcription factors (n=5 experiments, Foxp3 n=3) in Th1, Th17, and Treg over 5 days of skewing. J) Cytokine production in cells treated with vehicle or CB839. K) Average percent change of cytokine production in CB839-treated Th1, Th17, and Treg cells versus vehicle (n=7 experiments). L) Transcription factor expression of Th1, Th17 and Treg in Wild type cells treated with vehicle (black) or CB839 (green) over 5 days. M) Average percent change of transcription factors in CB839-treated Th1, Th17, and Treg cells versus vehicle (n=9 experiments, Foxp3 n=3).

FIGS. 12A-E examine gene expression and chromatin accessibility in GLS-deficient T cells. CD4+ T cells from wild type animals were differentiated over 5 days in Th1 and Th17 skewing media with or without CB839. A) Intracellular 2-oxoglutarate metabolite levels (normalized to vehicle of each subset. B) 2-Hydroxyglutarate metabolite levels as in A. C) Venn diagram of ATAC-Seq total changed peaks (open or closed) across differentiated Th1 (green) and Th17 (grey) cells treated with CB839. D) Ingenuity pathway analysis from FIG. 6D from Th1 cells in Cell Survival and Inflammatory response (left) and Lipid Metabolism (right) (green—downregulated, red, upregulated, as compared to vehicle treated). E) Motif analysis of the promoter regions with significantly changed peaks in Th1 and Th17 cells (more open, top, more closed, bottom). Sequences shown include Th1 more open with transcription factor AP-1 (SEQ ID NO:1); Th1 more open with ETS (SEQ ID NO:2); Th1 more open with IRF (SEQ ID NO:3); Th1 more open with CTCF (SEQ ID NO:4); Th1 more closed with ETS (SEQ ID NO:5); Th1 more closed with CTCF (SEQ ID NO:6); Th1 more closed with Runx (SEQ ID NO:7); Th1 more closed with Nrf (SEQ ID NO:8); Th17 more open with ETS (SEQ ID NO:9); Th17 more open with Runx (SEQ ID NO:10); Th17 more open with CTCF (SEQ ID NO:11); Th17 more open with AP-1 (SEQ ID NO:12); Th17 more closed with AP-1 (SEQ ID NO:13); Th17 more closed with CTCF (SEQ ID NO:14); Th17 more closed with ETS (SEQ ID NO:15); and Th17 more closed with IRF (SEQ ID NO:16).

FIGS. 13A-H show that GLS1 inhibition affects epigenetic landscape of Th1 and Th17 cells, leading to differential transcriptional and mTORC1 pathway signaling. (A-H) Wild type CD4+ T cells differentiated in Th1 and Th17 skewing conditions in the presence of vehicle or CB839. A) Top 200 modified genes from RNA-Seq compared to vehicle (Log2Fold>0.5, P<0.05) in Th1 (left) and Th17 (right). Particular genes of interest highlighted. B) Numbers of genes with more open (blue circles) and more closed (orange circles) chromatin peaks with CB839 as determined by ATAC-Seq. C and D) Cytokine production in Th1 (C) and Th17 (D) skewing conditions dosed with vehicle, CB839, or CB839 + dimethyl-oxoglutarate, a cell-permeable α-KG analog (representative of n=2 experiments). E) Average H3K4 trimethylation expression (left) and H3K27 trimethylation expression (right) in Th1 and Th17 cells (n=3/group, representative of 4 experiments). F) Example ATAC-Seq traces of IFNγ in Th1 and HIF1∝ in Th17 skewing conditions (left) and expression fold change with P value of RNA-Seq (right) (representative of n=3 traces/group). G) phospo-S6 expression after 5 days in Th1 or Th17 skewing conditions. H) Cytokine production of CD4+ T cells in Th1 skewing conditions in the presence of vehicle (top) or CB839 (bottom), and Rapamycin or No IL2 after 3-day split.

FIGS. 14A-B examines signaling pathways in in GLS-deficient T cells. A) Normalized message counts from RNA-Seq described in FIG. 6F, highlighting mTOR pathway targets. B) mTOR target protein expression by western blot from wild type CD4+ T cells differentiated in Th1 or Th17 skewing media with or without CB839 for 5 days. Actin control. Representative of n=2 experiments.

FIGS. 15A-G show that GLS KO T cells protect from Graft Versus Host Disease, fail to eliminate B cell leukemias. Temporary ex vivo CB839 treatment enhances T cell persistence in Vaccinia challenge and CAR T cell treatment. (A-D) Chronic Graft versus Host Disease (cGvHD) induced in C57BL6 animals with donor bone marrow and either GLS wild type (WT) or GLS^{fl/fl}CD4-CRE (GLS KO) T cells. A) Bodyweights measured pre- and post-transplant. B) Hematoxylin and eosin (H&E) stained lung sections focusing on bronchioles. C) Average histopathological scores from sections from B (n=5 animals, representative of two experiments). D) Percent cytokine producers from peripheral lymph node cells stimulated with PMA/ionomycin for 5 hours. (E and F) CAR T cell experiments. E) CD19+ B leukemic B cells per uL of blood at day 14 (left) and Day 28 (right) from C57BL6 mice injected with T cells isolated GLS WT or GLS KO spleens and transfected with Chimeric Antigen Receptor (CAR) (m19-28-Z) or control (m19-delta-Z). G) In vivo CAR T cell counts from transfected wild type T cells dosed with vehicle (GFP) or CB839-treated CAR T cells ex vivo and injected into recipient C57BL6 mice at week 4, and H) B cell counts from these same animals at week 4. Vaccinia viral response in wild type C57BL6 animals. Wild type CD8+ T cells were isolated and activated in vitro with hgp100₂₅₋₃₃-VV dosed with vehicle or CB839.

FIGS. 16A-E show cGVHD lung function readouts at day 49 in B10.BR animals. A) Lung function (read out of Bronchiolitis Obliterans) in recipient mice injected with T cell depleted bone marrow and either WT CD4+ or GLS KO CD4+ T cells from spleen. B) Chimeric Antigen Receptor (CAR) in blood of recipient mice injected with T cell depleted bone marrow and either WT or GLS^{fl/fl}CD4-CRE (GLS KO) T cells from spleen at day 14 (left) and day 28 (right). C) CAR in lymph nodes of recipient mice injected with T cell depleted bone marrow and either WT CD4+ or GLS KO CD4+ T cells from spleen at day 42. D) CD19+ B cells in blood 4 weeks after injection of T cells activated and transfected with m19-28-Z or control m19-delta-Z with CB839 (green) or without (black). E) pmel transgenic Ly45.1+ CD8+ T cells in blood of recipient animals, 7 days after initial ex vivo activation with or without CB839 and IV injection.

FIG. 17 contains graphs showing that GLS deficiency improves lung function.

FIG. 18 contains graphs showing that GLS deficiency alters lymphocyte numbers and percentages.

FIGS. 19A-D show that activated T cells rely on both glucose and glutamine to sustain cell metabolism. A) Metabolites extracted for mass spectrometry and presented as fold change from naïve in T cells stimulated for 16 hours (S) or naïve (N) conditions (*p<0.05, one-way ANOVA). B) Oxygen Consumption Rate (OCR) assayed from naïve CD4 cells from WT mice stimulated for 3 days on αCD3/CD28, injected with drug described (top). OCR at timepoint 200 min (bottom, ***p<0.001, one-way ANOVA). C-D) Abundance of metabolites (left, **p<0.01, unpaired t-test) and fractional labeling (right, *p<0.05, one-way ANOVA) of stimulated CD4+ T cells in the presence of CB839 and 13C-glucose for glutaminolytic intermediates (C) and TCA intermediates (D). Mean +/− Standard Deviation shown from n=3 replicates. Also see FIG. 20.

FIGS. 20A-F show that conversion of glutamine to glutamate contributes to T cell metabolism. A) Relative expression of glutamine pathway genes, data from Immgen (immgen.org). B) Relative ratio of glutamate:glutamine metabolite levels normalized to IL-7 (naïve, N) αCD3/CD28 (stimulated, S) normalized to naïve in wild type CD4+ T cells. (C-F) Additional intracellular metabolite abundance (left) and fraction labeled from 13Cglucose (right). C) Amino acids Serine, alanine, and glycine. D) Glycolytic intermediates G6P, F16BP. E) Lactate and Pyruvate. F) Nucleotide precursor Ncarbamoyl L-aspartate (average of n=3 replicates/group). Means +/− Std dev, (total abundance, left, ***p<0.001, student's t test; fractional labeling, right, ***p<0.001, one way ANOVA).

FIGS. 21A-F show that Th1 and Th17 cells differ in their use of glutaminolysis and GLS-deficiency is distinct from glutamine deficiency. A) Metabolite fold change from naïve in wild type CD4+ cells maintained in IL-7 (N), or differentiated for 5 days into Th1 (1), Th17 (17), or Treg (R) cells (*p<0.05, one-way ANOVA). B) Cytokine production from Th1 (top) and Th17 (bottom) differentiated T cells in the presence of glutamine (left), absence of glutamine (middle), or presence of GLS1 inhibitor CB839 (right) (representative of n=3 replicates). C) Proliferation of Cell Trace Violet (CTV) labeled T cells stimulated and differentiated in Th1 or Th17 conditions with (black lines) or without (red lines) glutamine after 3 and 5 days of culture. D) Same as in (C), but with vehicle (black lines) or CB839 (green lines) (representative of n=3 replicates). E) Foxp3 expression in CD4 T cells activated in Th1 or Th17 skewing conditions in glutamine deficient (red, left) conditions or in the presence of CB839 (green, right) (representative of n=3 replicates). F) Heat map (left) and principle component analysis (right) of metabolites from Th1 and Th17 cells with or without CB839 (n=3 replicates/group). Also see FIG. 22 and Table 2.

FIGS. 22A-G show glutamine and the role that GLS plays in Th1 and Th17 cell metabolism. A) Relative ratio of intracellular metabolites glutamate:glutamine from CD4+ T cells in Th1, Th17, and Treg skewing conditions normalized to naïve (average n=3 replicates/group). B) Immunoblot of GLS protein (top) and actin control (bottom) in T cells after five days in Th1 and Th17 skewing conditions. C-E) Normalized counts of message from RNA-Seq. C) Gls enzyme RNA expression from RNA-Seq from FIG. 21D. Gls2 expression from RNA-Seq from FIG. 21D on the same scale as Gls expression (left) and in smaller scale (right). For all RNA-Seq expression data, P values are determined from RNA-Seq analysis, all groups run in triplicate. D) Glud1, Got1, and Got2 expression as in (C). E) Pcx RNA expression as in (C) (All p values from defSeq2 program, n=3 replicates/group). F) Uptake (positive numbers) and secretion (negative numbers) of metabolites in CB839 treated wild type CD4+ T cells in Th1 and Th17 skewing conditions as measured by Nuclear Magnetic Resonance (NMR) (average of 3 replicates, ***P<0.001, unpaired t-test). G) Fluorescence of DCFDA dye by flow cytometry, representative histograms (left) and average of n=3 replicates (right, ***p<0.001, student's t-test) of vehicle or CB839-treated T cells in Th1 and Th17 skewing conditions.

FIGS. 23A-M show that glutaminase (GLS) is dispensable for T cell homeostasis, but constrains development of a Th1 -like phenotype. A) Immunoblot (left) and genomic DNA (right) in isolated Pan T cells (CD4+ and CD8+ ) from GLSfl/fl CRE+(GLS KO) and littermate wild type controls (WT). B) Cell counts (left) and percent of total splenocytes (right) from WT and GLS KO animals. No significance vs wild type, one-way ANOVA (n=3 animals/group). C and D) Flow cytometry analysis of T cell activation markers and cell size of CD4+ T cells (C) freshly isolated from WT and GLS KO T spleens or (D) activation markers and proliferation of WT and GLS KO CD4+ T cells activated on αCD3/CD28 over 48 hours (representative of n=3 replicates). E) Flow cytometry analysis of CD44 in CB839- or vehicle-treated T cells activated on αCD3/CD28 at day five (representative of n=3 replicates). F-K) Naive CD4+ T cells activated without cytokines over three days, split with IL-2, then stimulated to measure cytokines on day five. F) Cytokine production of wild type and GLS KO T cells. G) Average percent total IFNγ +producers (left), percent double positive IFNγ +IL2+ producers (middle), and the median fluorescence intensity (MFI) (right) of all IFNγ +cells in (F) (***p<0.001, unpaired t-test). H) Tbet protein expression in WT, GLS KO, and isotype control T cells. Representative of n=2 experiments. I-K) Same as in F-H, except with GLS inhibitor CB839 and vehicle. L and M) CD8+ T cells from WT or GLS KO animals activated on αCD3/CD28 + IL2 for five days. L) Expression of CD8+granzyme B protein at day 5, left, and average of granzyme B MFI signal, right (**p<0.01, student's t test, n=3 replicates/group). M) Tbet protein expression in WT, GLS KO, and isotype control (representative of n=2 experiments). Also see FIG. 24.

FIGS. 24A-N show that GLS-deficiency does not alter resting T cell phenotype but enhances Th1 and CD8+ T cell differentiation and cytokine production. A) Extracellular

Acidification Rate (ECAR) of naïve CD4+ T cells treated with vehicle or CB839 as measured by Seahorse (n=4 replicates/group). B) Average MFI of forward scatter (FSC) in activated CD8+ WT and GLS KO T cells (***p<0.001, student's t test, replicates of n=3/group). C) Viability by propidium iodide staining at day 3 and day 5 of WT T cells in activation condition with no cytokines (* * *p<0.001, student's t test, average of n=3 replicates). D-F) 2W peptide immunization of WT and GLS KO. D) Percent 2W-MHC II tetramer+ and CD44+ T cells by flow cytometry in both spleen and inguinal lymph nodes eight days after immunization with 2W antigen+CFA (right) or PBS control (left) in WT and GLS KO animals. E) Average count of CD44+ Tetramer+T cells as in (D) (p>0.05, student's t-test). F) IFNγ protein expression by flow from CD44+ MHC II tetramer+ T cells isolated from WT and GLS KO spleen and lymph nodes. G) Homeostatic proliferation of WT and GLS KO CD4/CD8+ T cells stained with cell trace violet (CTV) and injected into RAG1 KO recipient mice after five days (representative of n=5 replicates/group). H) Cell counts of CD8+ T cells from WT and GLS KO animals activated on αCD3/CD28+ IL2 for five days (**p<0.01, student's t-test). (I-O) CD8+ T cells activated αCD3/CD28+ IL2 for five days in the presence of CB839 or vehicle. I) Representative FACs plots of granzyme B producing cells. J) Perforin MFI (left) or granzyme B MFI (right) (***p<0.001, student's t-test). K) Representative Tbet expression. L) Average transcription factor expression (***p<0.001, student's t-test, n=3 replicates). M) Ki67 expression. N) Percent Lag3+ and PD1+ T cells as in (I). (*p<0.01, **p<0.01, student's t test, average of n=3 replicates).

FIGS. 25A-K show that GLS specifies Th1 and Th17 differentiation and metabolism. A-D) Naive CD4+ T cells from WT and GLS KO T cells differentiated in Th1, Th17, or Treg skewing media over five days. (A) IFNγ and IL2 production in Th1 skewing conditions (top) and IL-17 production in Th17 skewing conditions (bottom) (representative of n=3 replicates/group). B) Average percent change cytokine producers in Th1 and Th17 cells from WT (*p<0.05, paired t-test, average of n=5 experiments). C) Transcription factor expression of Th1, Th17, and Treg cells in WT (black) and GLS KO (red) (representative of n=3 replicates/group). D) Average percent change from WT of transcription factors (n=5 experiments, Foxp3 n=3 experiments, **p<0.01, one-sample t test,) in GLS KO T cells. (E-K) WT CD4+ T cells differentiated in Th1 or Th17 conditions in the presence of vehicle or CB839 over five days. E) Percent of Th1 cells producing IFNγ, IL2, and TNFα at day 5 (**p<0.01, unpaired student T test, n=3 replicates/group. NS=no stim). F) Median Fluorescence Intensity of inhibitory receptors (***p<0.001, two-way ANOVA). G) Fold change of metabolites from T cells differentiated in Th1 and Th17 conditions in the presence of CB839 relative to vehicle by mass spectrometry over five days. H) 3H-2-deoxyglucose uptake in Th1 and Th17 skewed T cells at day 3 (left) and day 5 (right) (***p<0.001, student's t test, n=3 replicates/group). I) Extracellular Acidification Rate (ECAR) of Th1 and Th17 skewed T cells at day 5 as in (H) (**p<0.01, student's t test). J) Fold change of Tbet (Th1 ) or RORyt (Th17) protein levels and (K) cell size in CB839-treated cells normalized to vehicle from same experiment as (G). Also see FIG. 26.

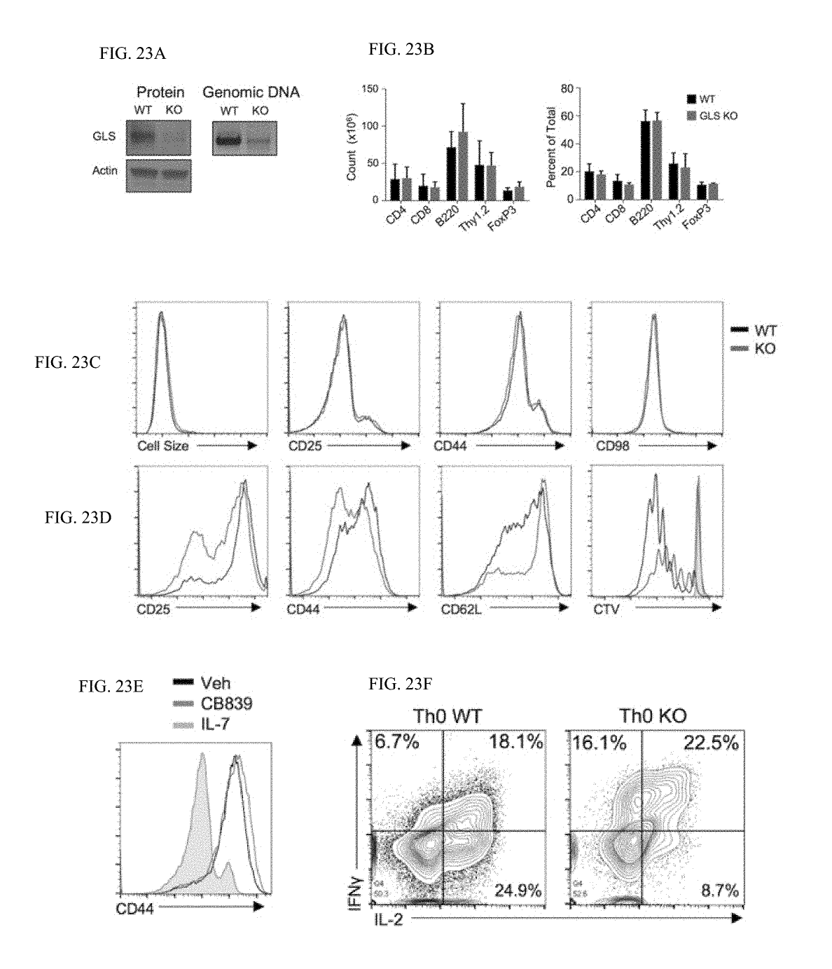

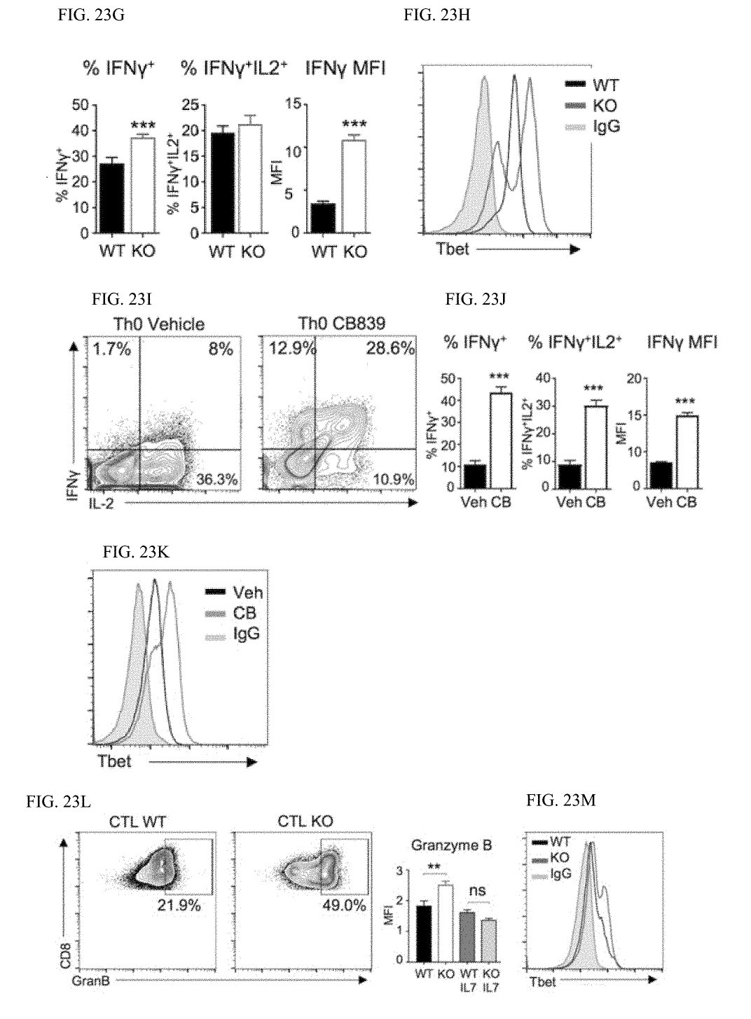

FIGS. 26A-I show that naïve CD4+ T cells from WT differentiated in Th1, Th17, or Treg skewing media over five days in the presence of CB839 or vehicle as in FIG. 25A. A) IFNγ and IL2 production in Th1 skewing conditions (top) and IL-17 production in Th17 skewing conditions (bottom) (representative of n=3 replicates/group). B) Percent change cytokine producers in Th1 and Th17 cells from vehicle (Th1, Th17 n=9 experiments, ***p<0.001, student T test). C) Transcription factor expression in wild type cells treated with Vehicle or CB839 (Tbet and RORyt, n=9 experiments, Foxp3 n=3 experiments). D) Average percent change from WT of transcription factor expression (Th1, Th17 n=7 experiments, Treg n=3 experiments, ***p<0.001, one-sample T test). E) Representative Klrg1 protein expression (F) average Klrg1 and CD279 expression (***p<0.001, student's t-test). (G-H) Metabolites in glycolysis (H) and Tricarboxylic Acid cycle (I) as in FIG. 23I-J (average of 3 replicates/group fold change from vehicle). I) Total RNA extracted from cells as in (A) at day 3 and day 5 (representative of n=2 experiments).

FIGS. 27A-K show that Th17 and Th1 cells differentially rely on GLS-mediated ROS neutralization and production of α-ketoglutarate to maintain chromatin (A-D) WT CD4+ T cells differentiated in Th1 or Th17 conditions in the presence of vehicle or CB839 over five days. A) Cytokine production in Th1 (top) and Th17 (bottom) skewing conditions dosed as indicated (representative of n=3 replicates). B) Average IFNγ +only producers (left) and average IFNγ +IL2+ producers (right) as in (A). C) Average protein expression of Tbet as in (A). D) Average IL-17A producers in Th17 skewing media (left) and average RORyt expression (right) (***p<0.001, one-way ANOVA, n=3 replicates/group). E-F) Global H3K27 trimethylation normalized to total H3 by flow cytometry. E) Average H3K27 trimethylation expression at Day 3. F) Same as (E), but at Day 5 (***p<0.001, student's t test, n=3 replicates/group). G) Average cytokine producers of skewed CD4+ T cells in the presence of CB839, JMJD3 inhibitor GSKJ4, or CB839+ GSKJ4 (CB+J4) at day 5 (**p<0.01, one-way ANOVA, n=3 replicates/group). H-I) WT CD4+ T cells differentiated in Th17 conditions as indicated. H) Percent IL17A+producers (left) and protein expression of RORyt (right). I) Average expression of H3K27me3 normalized to total H3 as in (H) (**p<0.01, one-way ANOVA, n=3/group). J) Number of loci with more (blue circles) and less accessible (orange circles) chromatin peaks with CB839 as determined by ATAC-Seq (n=3 replicates/group). K) Example ATAC-Seq traces of IFNγ in Th1 and IL17 gene locus in Th17 skewing conditions (representative of n=3 traces/group). Also see FIG. 28.

FIGS. 28A-H show that GLS deficiency differentially affects Th1 and Th17 T cells and modifies epigenetic landscape. A-B) Metabolite levels normalized to vehicle of each subset (A) Intracellular α-ketoglutarate metabolite levels and (B) 2-Hydroxyglutarate metabolite levels as in A (** P<0.01, unpaired t-test). C) MFI of H3K4me3 in Th1 and Th17 cells (***P<0.001, Two-way ANOVA, n=3 replicates/group). D) Percent total IFNγ+ producers in Th1 skewing conditions (***p<0.001, one-way ANOVA). E) MFI of H3K27me3 in Th1 skewing conditions (***p<0.001, one-way ANOVA). F) Venn diagram of ATAC-Seq total changed peaks (either open or closed). G) Ingenuity pathway analysis of altered ATACseq peaks from promoter regions in Th1 cells for Cell Survival and Inflammatory response (green—downregulated, red, upregulated, relative to vehicle treated). H) Motif analysis of the promoter regions with significantly changed peaks in Th1 and Th17 cells. Sequences shown include Th1 more open with transcription factor AP-1 (SEQ ID NO:1); Th1 more open with ETS (SEQ ID NO:2); Th1 more open with IRF (SEQ ID NO:3); Th1 more open with CTCF (SEQ ID NO:4); Th1 more closed with ETS (SEQ ID NO:5); Th1 more closed with CTCF (SEQ ID NO:6); Th1 more closed with Runx (SEQ ID NO:7); Th1 more closed with Nrf (SEQ ID NO:8); Th17 more open with ETS (SEQ ID NO:9); Th17 more open with Runx (SEQ ID NO:10); Th17 more open with CTCF (SEQ ID NO:11); Th17 more open with AP-1 (SEQ ID NO:12); Th17 more closed with AP-1 (SEQ ID NO:13); Th17 more closed with CTCF (SEQ ID NO:14); Th17 more closed with ETS (SEQ ID NO:15); and Th17 more closed with IRF (SEQ ID NO:16).

FIGS. 29A-H show that GLS inhibition alters gene expression to sensitize Th1 cells to IL2 activation of mTORC1. A) Top 200 modified genes from RNA-Seq compared to vehicle (Log2Fold>0.5, p<0.05) in Th1 (left) and Th17 (right) (n=3 replicates/group). B and C) Phospho-S6 expression on day 5 in Th1 and Th17 conditions as indicated with or without CB839 or IL2 2 at concentrations shown (ng/mL) at day 3 (***p<0.001, student's t test, n=3 replicates). D) Cytokine production in Th1 skewing conditions in the presence of vehicle (top) or CB839 (bottom) after five days, under no IL2 conditions or with IL2+ mTOR inhibitor rapamycin added on day 3 (representative of n=3 replicates). E) Phospho-S6 protein expression (left), average pS6 MFI (middle), percent IFNγ+IL2+ or IL2+ cells (right) in CD4 T cells in Th1 skewing conditions and infected with control- or PIK3IP1-expressing retrovirus with CB839-treatment. (middle: *p<0.05, student's t-test, right: *p<0.05, two-way ANOVA, n=3 replicates/group). F) Protein expression of phospho-S6 (left) and IFNγ (right) in activated Cas9-transgenic CD4+ T cells transduced with retrovirus containing control guide RNA, or guide RNAs targeting PIK31P1. G and H) Wild type CD4+ T cells activated and treated with PIK3IP1 antibody or IgG control antibody over 3 days. Protein expression of phospho-S6 (left), and average MFI of pS6 (right, *p<0.05, one-way ANOVA). H) Protein expression of activation markers of control- or PIK3IP1 antibody-treated T cells upon stimulation (no CB839) (representative of n=3 replicates). Also see FIG. 30 and Table 3.

FIGS. 30A-I show that Th1 cells are sensitive to mTOR signaling in GLS deficiency. A) Left: Percent IFNγ +producers in Th1 skewing conditions treated with or without CB839 and indicated levels of IL-2 (ng/mL). Right: Tbet protein expression as in left. (***p<0.001, student's t-test). B) Myc protein expression in WT and GLS KO CD4+ T cells in Th1 and Th17 skewing conditions (representative of n=3 replicates). C) MFI of H3K27me3 normalized to total H3 of CD4+ T cells in Th1 skewing conditions with indicated IL2 with or without CB839 (***p<0.001, one-way ANOVA, n=3 replicates/group). D and E) phospho-S6 protein expression measured by flow cytometry (D) in IL2 and IL2 depleted conditions with or without rapamycin (**p<0.01, one-way ANOVA compared to vehicle of each group, n=3 replicates/group) or (E) pS6 expression in Th0 (left) and CD8+ CTL cells (right) (***p<0.001 student's t-test, n=3 replicates/group). F) Normalized message counts from RNA-Seq described in FIG. 29A, highlighting PI3K/Akt/mTOR pathway targets (***p<0.001, p values obtained from defSeq2 program). G) PIK3IP1 protein expression in Wild Type CD4+ T cells in Th1 skewing conditions in the presence of CB839 infected with PIK3IP1 expression plasmid (representative of n=3 replicates). H) PIK3IP1 protein expression in CAS9-expressing CD4+ T cells in Th1 skewing conditions with guide RNAs targeting PIK3IP1 (CRISPR KO). I) Percent naïve cells in control or PIK3IP1 antibody-treated activated T cells (left) and CD25 expression (right) (*p<0.05, student's t-test, n=3 replicates/group).

FIGS. 31A-J show that GLS is essential for T cell-mediated inflammation but transient inhibition can augment T cell responses. A-C) Airway inflammation in cGvHD following transfer of WT or GLS KO T cells. A) Hematoxylin and eosin stained lung sections focusing on bronchioles. B) Average histopathological scores from sections from (A) (*p<0.05, unpaired T test, n=5 animals/group). C) Percent cytokine producers from peripheral lymph node cells stimulated with PMA/ionomycin for 5 hours from GvHD mice (BM: n=8, WT n=5, KO n=7, *** p<0.001, unpaired T test). D) Bodyweights from T cell adoptive transfer Inflammatory Bowel Disease (IBD) model in which RAG1 KO mice injected with WT and GLS KO naïve CD4+ T cells and induced for IBD with piroxicam (# p<0.05, p<0.01, two-way ANOVA, WT n=6, GLS KO n=8, data presented as mean±Standard Error (SEM)). E-F) T cells from WT and GLS KO infected to express CAR T cell constructs and injected into recipient mice. CAR—(no infection), A=m19-delta-ζ, 28-ζ=m19-28-ζ. E) CD19+ B cells per μL of blood at day 14 (left) and Day 28 (right). F) Same as in E, but at day 42 (***p<0.001, one-way ANOVA). G) CAR-T cell numbers on day 28 following transfer of CAR-T cells treated with vehicle or CB839 prior to transfer to recipient mice (WT no CAR: n=2 animals, all others n=5-6 animals, ***p<0.001, one-way ANOVA). H) Numbers of CD19+ Eμ-Myc lymphoma cells after 48 hours culture with indicated ratios of CAR-T cells (*p<0.05, student's t-test, average of n=3 replicates). I) Counts of total CD8+ cells in response to hgp10025-33-expressing vaccinia virus collected from tail vein after indicated time (*p<0.05, two-way ANOVA, n=5/group). (J) Counts of total CD8+ T cells in spleen (left) and lymph node (right) after 38 days and re-challenge with hgp10025-33-expressing vaccinia virus (Vehicle n=5 animals, GLS inhibitor n=4 animals, *p<0.05, two-way ANOVA). Also see FIG. 32.

FIGS. 32A-E show that GLS is essential in vivo for inflammation but transient GLS inhibition does not prevent CAR-T cell-mediated. A-B) cGVHD in C57BL6 animals as in FIG. 31A. A) Bodyweights of recipient mice injected with T cell depleted bone marrow and either WT CD4+ or GLS KO CD4+ T cells from spleen. n=9 animals/group (**p<0.01, one-way ANOVA). B) Lung physiology measurements (read out of Bronchiole Obliterans) from (A) (***p<0.001, one-way ANOVA). C) Percent of CD4+ T cells (left), CD4+ counts, IL17+ counts, IL4+, and IFNγ+ counts in WT and GLS KO mice immunized with PBS or house dust mite antigen (HDM) over 1 days (*p<0.05, student's t test). D) Percent IFNγ+, IFNy MFI, or percent IL17+, and IL17A MFI in mesenteric lymph nodes collected from RAG1 KO mice injected with wild type or GLS KO naïve CD4 T cells in IBD (*p<0.05, student's t test). E) Frequency of CD19+ B cells in blood 4 weeks after injection of T cells activated and infected with CAR T cell construct 28-ζ or control delta-ζ with or without GLS inhibitor (***p<0.001, one-way ANOVA).

FIG. 33 contains graphs showing that metformin (Met), 6-Diazo-5-Oxo-L-Norleucine (DON), and combinations of Met+DON improved pulmonary function.

FIG. 34 contains graphs showing that DON treatment reduces lymphocytes.

FIG. 35 contains graphs showing that DON treatment reduced GC B cells and increased TFR frequencies.

### DETAILED DESCRIPTION

Provided herein methods and materials for treating or preventing GVHD. For example, this document provides methods and materials for using one or more glutaminolysis inhibitors to treat or prevent GVHD. In some cases, the methods and materials described herein can reduce morbidity and/or mortality in subjects who undergo allogeneic hematopoietic stem-cell transplantation.

When treating and/or preventing GVHD as described herein, the GVHD can be any type of GVHD. GVHD can be acute graft versus host disease (aGvHD). GVHD can be chronic graft versus host disease (cGvHD). GVHD, and particularly, cGVHD, is a significant cause of morbidity and mortality after hematopoietic stem cell transplantation, particularly after allogeneic hematopoietic stem cell transplantation.

When treating and/or preventing GVHD as described herein, the GVHD can be associated with any appropriate transplant. Examples of transplants that GVHD can be associated with include, without limitation, organ (e.g., heart, lung, kidney, and liver) transplants, tissue (e.g., skin, cornea, and blood vessels) transplants, and cell (e.g., bone marrow and blood) transplants. A transplant can include an allograft. A transplant can include an autograft. A transplant can include a xenograft.

In some cases, the methods and materials described herein can be used to reduce or eliminate one or more symptoms of GVHD. cGVHD can occur in the skin (e.g., rash, raised, or discolored areas, skin thickening or tightening), liver (e.g., abdominal swelling, yellow discoloration of the skin and/or eyes, and abnormal blood test results), eyes (e.g., dry eyes or vision changes), gastrointestinal tract (e.g., mouth, esophagus, stomach, intestines) (e.g., dry mouth, white patches inside the mouth, pain or sensitivity, difficulty swallowing, pain with swallowing, or weight loss), lungs (e.g., shortness of breath or changes on chest X-rays), neuromuscular system (e.g., fatigue, muscle weakness, or pain), or genitourinary tract (e.g., increased frequency of urination, burning or bleeding with urination, vaginal dryness/tightening, or penile dysfunction), which can result in individuals presenting with a wide variety of additional symptoms. For example, one or more glutaminolysis inhibitors can be administered to a subject identified as having GVHD, or identified as being likely to develop GVHD, to reduce one or more symptoms of GVHD.

In some cases, the methods provided herein can include identifying a subject (e.g., a mammal) as having GVHD. Any appropriate method can be used to identify a subject having GVHD. For example, cGVHD is most often diagnosed by the presence of a skin rash or by changes in the eyes or mouth. cGVHD can cause damage in the glands that produce tears in the eyes and saliva in the mouth, resulting in dry eyes or a dry mouth, and individuals can have mouth ulcers, skin rashes, or liver inflammation. Examples of methods that can be used to identify a subject having GVHD include, without limitation, physical examination (e.g., for observation of certain symptoms such as fever, skin rash, skin redness, skin itchiness, yellow discoloration of the skin, yellow discoloration of the eyes, dryness of the eyes, irritation of the eyes, nausea, vomiting, diarrhea, and abdominal cramping), biopsy (e.g., biopsy of the transplanted tissue), and/or laboratory tests (e.g., liver enzyme panels).

In some cases, the methods provided herein also can include identifying a subject (e.g., a mammal) as being at risk of developing GVHD. Any appropriate method can be used to identify a subject for risk of developing GVHD. For example, hematopoietic stem cell transplant (e.g., from a blood or bone marrow) from one individual to another, referred to as an allogeneic transplant (e.g., allogeneic hematopoietic stem cell transplant), can result in the recipient developing GVHD. Older individuals, individuals who have received a peripheral blood transplant (instead of a bone marrow transplant), and individuals who have received a transplant from a mismatched or unrelated donor have a greater risk of developing GVHD. In addition, individuals who have had aGVHD have a greater risk of developing cGVHD. cGVHD can appear at any time after allogeneic transplant, from several months to several years after transplant. Typically, cGVHD begins later after transplant and lasts longer than aGVHD. Examples of methods that can be used to identify a subject as being at risk of developing GVHD include, without limitation, identifying a subject as having an HLA (human leukocyte antigen) mismatch (e.g., an HLA match in which there are differences between the donor and the recipient subject), identifying a female subject as having recently been pregnant, and/or identifying a subject as being of advanced age.

In some cases, the methods and materials described herein can be used to treat or prevent one or more complications associated with GVHD. For example, cGVHD also can result in formation of scar tissue in the skin (e.g., cutaneous sclerosis), and joints, and damage to air passages in the lungs, resulting in bronchiolitis obliterans (BO) syndrome and/or fibrosis. cGVHD also results in a significantly increased risk of the subject developing infections. Following a blood or bone marrow stem cell transplant, individuals (also referred to as recipient subjects) can be administered one or more immunosuppressants (e.g., prophylactically) to lower the risk of developing GVHD. In addition, treatment options once a subject has been diagnosed with GVHD generally include administration of one or more immunosuppressants (e.g., a long-term immunosuppressive regimen). While immunosuppressants decrease the ability of donor T cells to initiate and maintain an immune response against the recipient, fungal, bacterial and viral infections are significant risks with any type of immunosuppressant regimen. Examples of complications associated with GVHD include, without limitation, BO syndrome, fibrosis, and infection. For example, one or more glutaminolysis inhibitors can be administered to a subject identified as having GVHD, or identified as being likely to develop GVHD, to treat or prevent infections (e.g., fungal bacterial, and/or viral infections).

In some cases, the methods and materials described herein can be used to improve pulmonary function in a subject. Pulmonary function can be assessed using any appropriate method. Examples of respiratory mechanics that can be measured to evaluate pulmonary function include, without limitation, compliance, elastance, resistance, oxygen consumption rate (OCR), and extracellular acidification rate (ECAR). Examples of methods that can be used to evaluate pulmonary function include, without limitation, spirometry, and lung volume measurement (e.g., body plethysmography and/or diffusion capacity). For example, one or more glutaminolysis inhibitors can be administered to a subject identified as having GVHD, or identified as being likely to develop GVHD, to reduce resistance, elastance, and/or compliance. For example, one or more glutaminolysis inhibitors can be administered to a subject identified as having GVHD, or identified as being likely to develop GVHD, to increase OCR and/or ECAR.

In some cases, the methods and materials described herein can be used to alter (e.g., increase or decrease) the number of lymphocytes in a subject. The lymphocyte can be any type of T cell. For example, the lymphocyte can be a T cell or a B cell. In cases where a lymphocyte is a T cell, the T cell can be any appropriate kind of T cell (e.g., T helper (Tₕ; e.g., CD4⁻) cells, effector T (T_{eff}) cells, and regulatory T (T_{reg}) cells such as follicular regulatory T (T_{FR}) cells and follicular helper T (T_{FH}) cells). In cases where a lymphocyte is a T cell, the methods and materials described herein can be used to alter the number of T cells in a subject and/or the frequency of T cells (e.g., the percentage of a particular type of T cell within the T cell population) in a subject. For example, one or more glutaminolysis inhibitors can be administered to a subject identified as having GVHD, or identified as being likely to develop GVHD, to decrease the percentage of Tₕ cells and/or the percentage of T_{reg} cells in a subject. For example, one or more glutaminolysis inhibitors can be administered to a subject identified as having GVHD, or identified as being likely to develop GVHD, to increase the percentage of T_{FR} and/or the percentage of T_{FH} cells within the Tₕ population a subject. In cases where a lymphocyte is a B cell, the B cell can be any appropriate kind of B cell (e.g., germinal center (GC) B cells). In cases where a lymphocyte is a B cell, the methods and materials described herein can be used to alter (e.g., decrease) the number of B cells in a subject and/or the frequency of B cells (e.g., the percentage of a particular type of B cell within the B cell population) in a subject. For example, one or more glutaminolysis inhibitors can be administered to a subject identified as having GVHD, or identified as being likely to develop GVHD, to decrease the percentage of GC B cells in a subject.

Any type of subject having GVHD or at risk for developing GVHD can be treated as described herein. In some cases, a subject can be a mammal. Examples of mammals that can be treated with one or more glutaminolysis inhibitors described herein (e.g., DON, CB839, and BPTES) include, without limitation, humans, non-human primates (e.g., monkeys), dogs, cats, horses, cows, pigs, sheep, rabbits, mice, and rats. For example, humans having GVHD or at risk of developing GVHD can be treated with one or more glutaminolysis inhibitors as described herein.

Once identified as having GVHD or as being at risk for developing GVHD, a subject (e.g., a mammal) can be administered or instructed to self-administer one or more (e.g., one, two, three, four, five, or more) catecholamine synthesis inhibitors described herein (e.g., natriuretic peptides and/or tyrosine hydroxylase inhibitors).

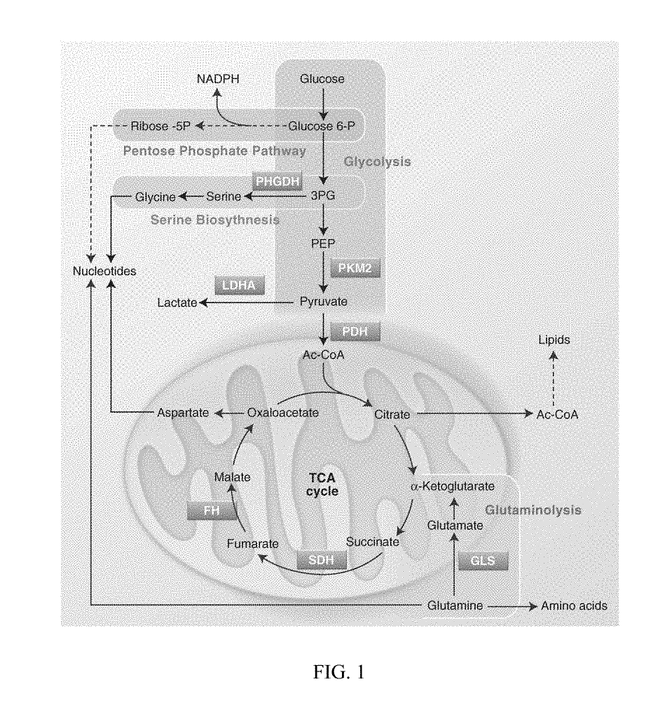

As used herein, a “glutaminolysis inhibitor” can be any agent that can disrupt (e.g., reduce or eliminate) the conversion of glutamine to alpha (α)-ketoglutarate (see, e.g., FIG. 1). For example, a glutaminolysis inhibitor can reduce or eliminate the amount of carbon that enters the tricarboxylic acid (TCA) cycle. In some cases, a glutaminolysis inhibitor can inhibit the conversion of glutamine to glutamate. In some cases, a glutaminolysis inhibitor can inhibit the conversion of glutamate to a-ketoglutarate. For example, a glutaminolysis inhibitor can inhibit an enzyme that catalyzes the conversion of glutamine to a-ketoglutarate. A glutaminolysis inhibitor can inhibit polypeptide expression or polypeptide activity of an enzyme that catalyzes the conversion of glutamine to α-ketoglutarate. A glutaminolysis inhibitor can be a small molecule. A glutaminolysis inhibitor can be a nucleic acid molecule designed to induce RNA interference (e.g., a siRNA molecule or a shRNA molecule), antisense molecules, and miRNAs). Examples of enzymes that catalyze the conversion of glutamine to α-ketoglutarate include, without limitation, inhibitors of glutaminase (GLS), glutamate dehydrogenase (G1DH), glutamate pyruvate transaminase (GPT; also called alanine transaminase (ALT)), and glutamate oxaloacetate transaminases (GOTs; such as GOT1 and GOT2). In some cases, a glutaminolysis inhibitor can inhibit polypeptide expression or polypeptide activity of GLS. Examples of compounds that can inhibit GLS include, without limitation, DON, CB839, and BPTES. For example, a glutaminolysis inhibitor can be DON.

In some cases, an inhibitor of GLS polypeptide expression or polypeptide activity can be readily designed based upon the nucleic acid and/or polypeptide sequences of GLS. Examples of GLS nucleic acids include, without limitation, the human GLS sequences set forth in National Center for Biotechnology Information (NCBI) GenBank® Accession Nos. AF110330 (Version AF110330.1), AF110331 (Version AF110331.1), and AF327434 (Version AF327434.1). Examples of GLS polypeptides include, without limitation, the human GLS polypeptides having the amino acid sequence set forth in NCBI GenBank® Accession Nos: AAF21934 (Version AAF21934.1), AAG47842 (Version AAG47842.1), and AAF21933 (Version AAF21933.1).

This disclosure describes methods of treating or preventing graft-versus-host disease (GVHD) in a subject by administering one or more glutaminolysis inhibitors described herein (e.g., DON, CB839, and BPTES) to the subject. One or more glutaminolysis inhibitors can be administered to a subject prior to the subject receiving a transplant. Additionally or alternatively, one or more glutaminolysis inhibitors can be administered to the subject concurrently with the transplant and/or at any time after they have received a transplant. As used herein, “transplant” typically refers to a blood or a bone marrow transplant such as, for example, an allogeneic blood or bone marrow transplant. Also additionally or alternatively, donor cells (e.g., donor T cells) can be contacted with one or more glutaminolysis inhibitors ex vivo prior to transplantation into the recipient.

One or more glutaminolysis inhibitors described herein (e.g., DON, CB839, and BPTES) can be formulated with a pharmaceutically acceptable carrier for delivery to an individual in a therapeutically-effective amount. The particular formulation and the therapeutically-effective amount are dependent upon a variety of factors including, but not limited to, the route of administration, the dosage and dosage interval of the one or more glutaminolysis inhibitors, the sex, age, and weight of the subject being treated, and the severity of the GVHD.

As used herein, “pharmaceutically acceptable carrier” is intended to include any and all excipients, solvents, dispersion media, coatings, antibacterial and anti-fungal agents, isotonic and absorption delaying agents, and the like, compatible with administration. The use of such media and agents for pharmaceutically acceptable carriers is well known in the art. Except insofar as any conventional media or agent is incompatible with a compound, use thereof is contemplated.

Pharmaceutically acceptable carriers are well known in the art. See, for example Remington: The Science and Practice of Pharmacy, University of the Sciences in Philadelphia, Ed., 21^{st} Edition, 2005, Lippincott Williams & Wilkins; and The Pharmacological Basis of Therapeutics, Goodman and Gilman, Eds., 12^{th} Ed., 2001, McGraw-Hill Co. Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, N.Y.

A pharmaceutical composition that includes a compound as described herein is typically formulated to be compatible with its intended route of administration. Suitable routes of administration include, for example, oral, rectal, topical, nasal, pulmonary, ocular, intestinal, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

For intravenous injection, for example, the composition may be formulated as an aqueous solution using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, optionally containing penetration enhancers, which are known in the art. For oral administration, a compound can be formulated in liquid or solid dosage forms, and also formulation as an instant release or controlled/sustained release formulations. Suitable dosage forms for oral ingestion by an individual include tablets, pills, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which can include fillers, disintegrants, binders (dry and wet), dissolution retardants, lubricants, glidants, anti-adherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (e.g., dextrose, sucrose, lactose), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. When a capsule is preferred over a tablet, the drug powder, suspension, or solution thereof can be delivered in a compatible hard or soft shell capsule.

One or more glutaminolysis inhibitors described herein (e.g., DON, CB839, and BPTES) can be administered locally or systemically. One or more glutaminolysis inhibitors described herein can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and the use of complexing agents. For administration by inhalation (e.g., via the mouth or nose), compounds can be delivered in the form of a solution, suspension, emulsion, or semisolid aerosol from pressurized packs, or a nebuliser, usually with the use of a propellant, e.g., halogenated carbons.

Compounds described herein also can be formulated for parenteral administration (e.g., by injection). Such formulations are usually sterile and, can be provided in unit dosage forms, e.g., in ampoules, syringes, injection pens, or in multi-dose containers, the latter usually containing a preservative. The formulations may take such forms as suspensions, solutions, or emulsions in oily or aqueous vehicles, and may contain other agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Polymers such as poly(lactic acid), poly(glycolic acid), or copolymers thereof, can serve as controlled or sustained release matrices, in addition to others well known in the art. Other delivery systems may be provided in the form of implants or pumps.

One or more glutaminolysis inhibitors described herein (e.g., DON, CB839, and BPTES) can be administered at least once a day (e.g., at least twice a day, at least three times a day, or more) to a subject suffering from GVHD or at risk of developing GVHD. For example, one or more glutaminolysis inhibitors can be administered to a subject for a short period of time (e.g., for one or a few days, for one or a few weeks), or one or more glutaminolysis inhibitors can be administered chronically (e.g., for several weeks, months or years) to a subject suffering from GVHD or at risk of developing GVHD.

One or more glutaminolysis inhibitors described herein (e.g., DON, CB839, and BPTES) can be administered in a therapeutically effective amount to a subject suffering from GVHD. Typically, a therapeutically effective amount is an amount that imparts beneficial effects without inducing any adverse effects. Toxicity and therapeutic efficacy of the one or more glutaminolysis inhibitors can be determined by standard pharmaceutical procedures in cell cultures or experimental animals, e.g., by determining the LD₅₀ (the dose lethal to 50% of the population), the ED₅₀ (the dose therapeutically effective in 50% of the population), and/or the LD₅₀/ED₅₀ ratio (the therapeutic index, expressed as the dose ratio of toxic to therapeutic effects).

One or more glutaminolysis inhibitors described herein (e.g., DON, CB839, and BPTES) can be administered to the subject at a dose of from about 0.5 mg to about 50 mg (e.g., from about 0.6 mg to about 50 mg, from about 0.8 mg to about 50 mg, from about 1 mg to about 50 mg, from about 1.2 mg to about 50 mg, from about 1.5 mg to about 50 mg, from about 2.5 mg to about 50 mg, from about 5 mg to about 50 mg, from about 10 mg to about 50 mg, from about 25 mg to about 50 mg, from about 35 mg to about 50 mg, from about 45 mg to about 50 mg, from about 0.5 mg to about 40 mg, from about 0.5 mg to about 30 mg, from about 0.5 mg to about 20 mg, from about 0.5 mg to about 10 mg, from about 0.5 mg to about 8 mg, from about 0.5 mg to about 5 mg, from about 0.5 mg to about 2.5 mg, from about 0.5 mg to about 1.3 mg, from about 0.5 mg to about 1 mg, from about 0.7 mg to about 40 mg, from about 1 mg to about 30 mg, from about 1.2 mg to about 20 mg, from about 1.3 mg to about 10 mg, from about 1.4 mg to about 5 mg, or from about 1.5 mg to about 3 mg) of the one or more glutaminolysis inhibitors per kilogram (kg) of the subject. For example, DON can be administered to the subject at a dose of about 1.6 mg/kg of the subject.

As used herein, “treating” refers to reversing, alleviating, or inhibiting the progression of GVHD, or one or more symptoms associated with GVHD and “preventing” refers to avoiding or precluding the development of GVHD or one or more of the symptoms associated with GVHD. It would be understood that the particular therapeutic endpoint(s) that determines whether or not treatment has been achieved (e.g., whether or not a patient has been treated) will depend upon how the GVHD manifests itself (e.g., the tissue or organs affected, the severity or acuteness of the disease, or the coexistence of more than one disease) in each subject. For examples of therapeutic and clinical guidelines for GVHD, see, for example, Lee et al. (2015, Biol. Blood Marrow Transplant., 21:984-999); Jagasia et al. (2015, Biol. Blood Marrow Transplant., 21:389-401); and Miklos et al. (2017, Blood, doi: 10.1182/blood-2017-07-793786).

Briefly, clinical cGVHD can involve not only classical acute GVHD (aGVHD) epithelial target tissues (e.g., GI tract, liver, skin, lung) but any other organ system including, without limitation, oral, esophageal, musculoskeletal, joint, fascial, hair and nails, ocular, lymphohematopoietic system and genital tissues. Eight organ systems (i.e., skin, mouth, eyes, gastrointestinal tract, liver, lungs, genital tract and fasciae/joints) evaluated for diagnosis are scored (range 0-3) for individual organ system severity and summed to calculate global cGVHD severity. Primary efficacy endpoints are best overall cGVHD response rate, which is defined as the proportion of all subjects who achieve a complete response (CR) or partial response (PR) (based on the 2014 NIH Consensus Panel). All subjects who have at least one response assessment are considered response-evaluable. Secondary efficacy end points include sustained response of ≥20 weeks, changes in corticosteroid requirement over time, and change in the Lee cGVHD Symptom Scale (self-reported). A decrease by ≥7 points is considered clinically meaningful and relates to improved quality of life.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Example 1

### Glutaminolysis and T Cell Responses

The tricarboxylic acid (TCA) cycle (also known as the citric acid cycle (CAC) or the Krebs cycle) that uses carbon as a source to generate biosynthetic precursors that are necessary for cells to proliferate. Carbon can enter the TCA cycle through the glutaminolysis (the conversion of glutamine to alpha-ketoglutarate) or through glucose-derived acetyl-CoA.

### Materials and Methods

### Mice

Mice were obtained from the Jackson laboratory or as described elsewhere (Young et al., 2011 PLoS One 6(8):e23205). GLS^{fl/fl} animals were obtained as embryonic stem cells from the KOMP and crossed to FLP transgenic animals to delete the Neo cassette. These progeny were then crossed with CD4-CRE transgenic mice to develop the GLS^{fl/fl} CD4-CRE (GLS KO). In all cases comparing wild type to GLS KO, sex-matched and age-matched littermates were used. All procedures were performed under appropriate IACUC-approved protocols.

### T Cell In Vitro Activation and Skew Experiments

T cell skew and activation: All T cell cultures were grown in RPMI 1640 supplemented with glutamine, HEPES, BME, and Pen/Strep unless otherwise noted. Naïve CD4 T cells were isolated from wild type animals (WT) and GLS^{fl/fl} CD4-CRE+ mice (GLS KO) and activated over various time points via anti-CD3/anti-CD28 antibodies plate bound. Non-stim CD4 samples were maintained using 10 ng/mL IL-7. For skewed experiments, naïve CD4 T cells from WT or KO animals were plated with subset-specific cytokines and stimulated with feeder layer of irradiated splenocytes. After 3 days, cells were split with fresh media and stimulated with 1:1500 IL-2 for a further 2 days. For intracellular cytokine stains, cells were re-stimulated using PMA/ionomycin in the presence of GolgiPlug (BD, Cat #: 555029) for 4 hours, then fixed and stained for intracellular subset-specific cytokines using BD Bioscience fix/perm kit (Cat #: 554714). For all other intracellular or intranuclear stains such as transcription factor, pS6, C-MYC, H3K4me3, H3K27me3, and total H3 protein, cells were removed from media, stained for surface markers, fixed, then stained for intracellular proteins using ebioscience fix/perm kit (Cat #s: 00-5223-56, 00-5123-43). Cell proliferation was assessed by staining naïve CD4+ cells with Cell Trace Violet proliferative dye (Invitrogen, Cat #: c34557).

### Sequencing Experiments

ATAC-Seq: Crude nuclei pellets were isolated as described elsewhere (see, e.g., Buenrostro et al., 2013 Nat Meth. 10(12):1213-1218) with modifications. Briefly, naïve CD4 T cells were skewed to Th1 and Th17 subsets in vitro with vehicle or in the presence of 500 nM CB839. At Day 5, T cells were re-isolated for CD4+ cells using Miltenyi CD4+ negative selection kit (Cat #: 130-104-454). 1×10⁵ cells were removed for nuclei extraction in ATAC-Seq lysing buffer. Cells were exposed to Tn5+ adaptor proteins from Nextera DNA for 30 min at 37° C. and immediately placed on ice. Transposed eluate was amplified via PCR using Nextera DNA preparation kit (Illumina, Cat #: FC-121-1030), NEBNext High-fidelity 2× PCR mix (New England Labs, Cat #: M0541), and multiplexed (Illumina, Cat #: FC-121-1011). Samples were purified using Zymo DNA cleanup kit (Cat #: D4011). QC of samples was run on bioanalyzer before being sent for sequencing. RNA-Seq: Th1 and Th17 cells skewed with or without CB839 were isolated as previously described and total RNA extracted (Qiagen RNEasy Mini kit, Cat #: 74104). RNA was sent to VANTAGE core at Vanderbilt University and sequenced on HiSeq 2500. n=3 for each sample was analyzed. Samples were analyzed using the R program DESeq2. GSEA was performed using MSigDB.

qPCR

T cells were isolated and purified as previously described. RNA was isolated using Qiagen RNEasy mini kits. RNA was converted to cDNA via high-capacity cDNA reverse transcription kit. PCX1 and PCX2 genes were designed using PrimerBank (pga.mgh.harvard.edu/primerbank/). qPCR run via SYBRGreen and Bio-Rad qPCR CFX96 Touch. mRNA levels were analyzed by calculating delta-delta CT from vehicle controls.

### Metabolic Assays

Glucose uptake assays were performed as described elsewhere (see, e.g., Macintyre et al., 2014 Cell Metab. 20(1):61-72). Naïve CD4+ T cells were differentiated into Th1 and Th17 cells, in triplicate, in the presence or absence of CB839 over 5 days and spun down after reisolation using CD4 kit as previously described. Cells were washed 2× in PBS, counted, then rested in 1 mL Kreb's Ringers HEPES (KRH) for 10 minutes. Cells were spun and resuspended to 5×10⁵ cells/50 uL KRH for glucose uptake assay. Briefly, cells were suspended in an oil bubble layered in KRH, and ³H-2-deoxyglucose was added to this bubble. Cells incubated for 10 minutes at 37° C. Immediately after incubation, reaction was quenched with 200 μM phloretin (Calbiochem, Cat #: 524488). Cells were spun, washed, and then resuspended in scint fluid for counting on Beckman-Coulter scintillation counter. Pathway analysis of altered metabolites was performed using Metaboanalyst 3.0 (metaboanalyst. ca/faces/home.xhtml).

### Seahorse

Experiments were carried out on Agilent Seahorse XF96 bioanalyzer (Agilent). Briefly, wild type CD4+ cells were isolated as previous and activated for 3 days on CD3/CD28 as previously described, or skewed to Th1 and Th17 subsets as described above. T cells were spun onto XF96 Cell-Tak (BD Bioscience, Cat #: 354240) coated plates and rested in Seahorse XF RPMI 1640 media supplemented with glutamine, sodium pyruvate, and glucose. For immediate metabolic response, CB839 and UK5099 were injected separately or in combination, and OCR and ECAR measured.

### Mass Spectrometry

¹³C-Glucose Activation: CD4 cells were activated on 5 μg/mL αCD3/CD28 for 3 days. At day 3, cells were pooled, washed 3× in PBS, and re-stimulated in presence of 1 μM CB839 or Vehicle (DMSO) and 11 mM ¹³C glucose (Cambridge Isotope Labs, Cat #: CLM-1396-1). Cells were incubated for 24 hours at 37° C., then scraped and combined in triplicate. Cells were rinsed with 0.9% saline and metabolites were extracted in methanol. Metabolites measured by LC-High-Resolution Mass Spectrometer (LC-HRMS) using a Q-exactive machine. The time-dependent glucose labeling pattern was modeled as with the following equation:

$$\frac{[ {X}^{*} ]}{{X}^{T}} = 1 - {e}^{- \frac{{f}_{X}}{{X}^{T}} t}$$

In which ^{[X*] is the concentration of labeled glucose, X}^{T} is the total concentration (both labeled and unlabeled) of glucose, f_{X} is the glucose production flux. This model was fit to glucose MIDs using the fit function in MATLAB to determine relative glucose production fluxes. Relative glucose pool sizes were estimated from MS signal intensities.

Differentiation: CD4 cells were isolated as previously described and differentiated in subset-specific medium (in triplicate) for 3 days, split at day 3 with new media and IL-2, then allowed to incubate a further 2 days. At day 5, wells were combined, cells washed lx in MACS buffer, re-isolated for CD4 via AutoMACS Pro automated magnetic separator (Miltenyi, Cat #: 130-092-545). Metabolites from Th1 and Th17 cells were extracted as described above.

### Statistical Analysis

Statistical analyses were performed with Prism software (GraphPad) using the student T-test, one-way ANOVA unless otherwise noted. Longitudinal data was analyzed by two-way ANOVA followed by Tukey's test and followed up with one-way ANOVA or T-test as specified. Statistically significant results are indicated (*p<0.05) and n.s. indicates select non-significant data. Error bars show mean±Standard Deviation unless otherwise indicated. RNA-Seq data were analyzed by DESeq2 in R.

### Results

### GLS and Glutaminolysis Contribute to T Cell Metabolism Upon Activation

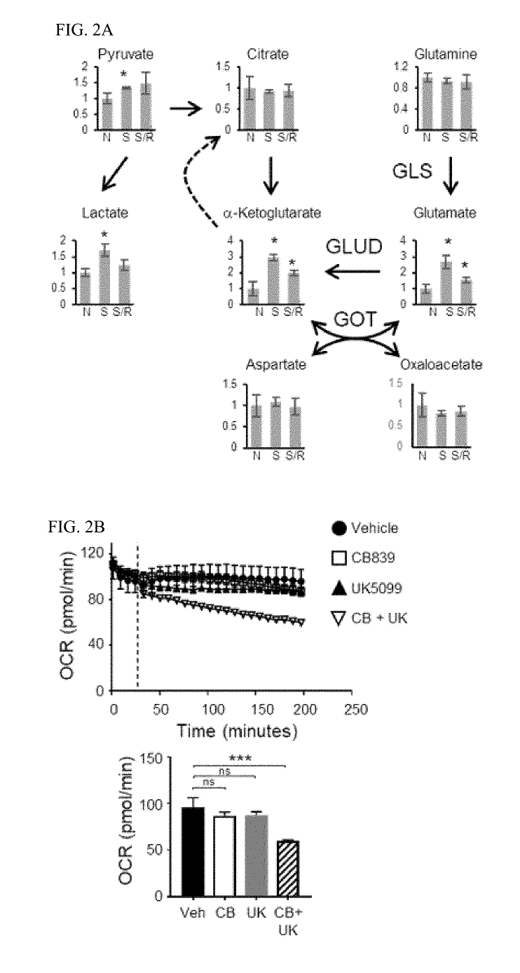

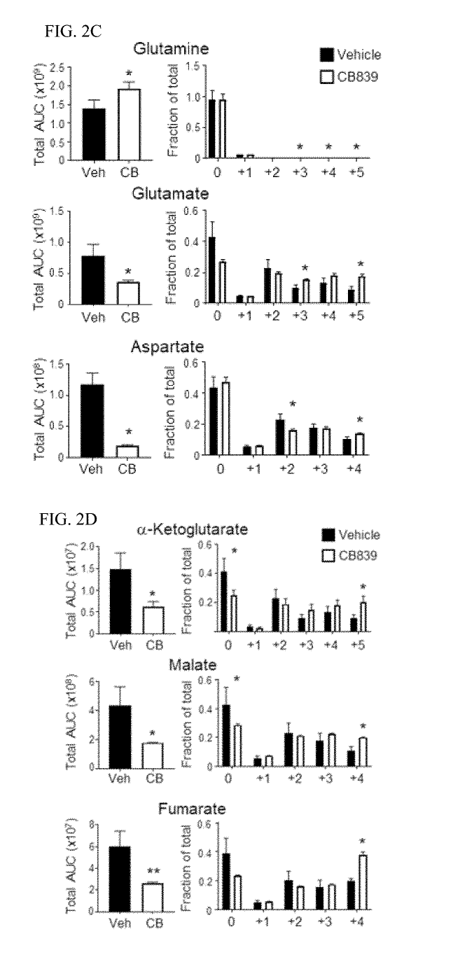

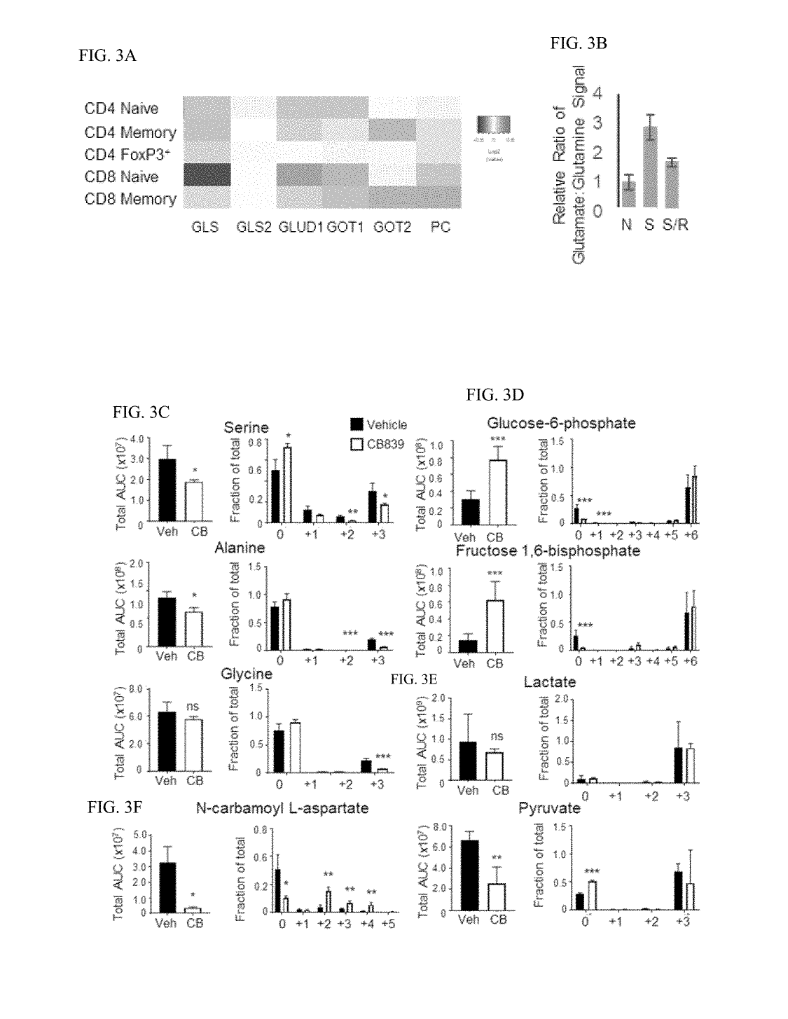

T cells have significant metabolic requirements during activation and proliferation that are met in part by glucose and glutamine. To determine the relative roles of glucose and glutamine, metabolites were measured following activation of CD4 T cells. In addition to increased lactate, glutamate and α-KG levels increased, suggesting elevated glutamine metabolism (FIG. 2A). Glutamate is primarily generated from glutamine by GLS or from α-KG and aspartate by GOT1, both of which are expressed in CD4 and CD8 T cells (FIG. 3A). The increased levels of both α-KG and glutamate and high ratio of signal from glutamine to glutamate (FIG. 3B), however, suggested GLS as a primary source of glutamate and α-KG production. To test this and determine the relative roles of GLS and glycolysis as fuels for mitochondrial metabolism, oxygen consumption of T cells stimulated overnight and treated with mitochondrial pyruvate carrier (UK5099) or GLS (CB839) inhibitors was measured. While neither UK5099 nor CB839 were sufficient on their own to reduce T cell respiration, the combination led to a progressive decrease in oxygen consumption (FIG. 2B). These data support an integrated metabolism in which stimulated T cells use glycolysis and GLS-dependent glutaminolysis.

To directly determine how inhibition of GLS affects glucose metabolism, CD4 T cells were stimulated in uniformly labeled ¹³C-glucose with or without CB839 and glucose derived carbons were traced. Inhibition of GLS led to increased intracellular glutamine and decreased glutamate (FIG. 2C). Aspartate levels also decreased significantly. Representation of glucose-derived ¹³C was increased in both glutamate and aspartate, demonstrating decreased overall levels, but a greater fraction of glucose contribution to synthesis of these amino acids. Additionally, the increased m+5 labelling in α-KG and glutamate implies that pyruvate conversion to oxaloacetate by pyruvate carboxylase was active. Serine and alanine abundance also decreased while glycine was unchanged with a decreased portion derived from glucose (FIG. 3C). TCA intermediates were also reduced in overall abundance, yet with increased fractional labeling with glucose-derived ¹³C (FIG. 2D). Glycolytic intermediates were more abundant upon GLS-inhibition, signifying elevated glycolysis (FIG. 3D). However, lactate levels and ¹³C-labeling were unchanged and pyruvate abundance decreased (FIG. 3E). Anabolic pathways were also affected, as the nucleotide precursor N-carbamoyl-aspartate was decreased (FIG. 3F). Altogether, these data suggest glucose metabolism was increased and a greater fraction entered the TCA cycle when GLS was impaired.

### CD4 T Cell Subsets Have Distinct Programs of Glutamine Metabolism

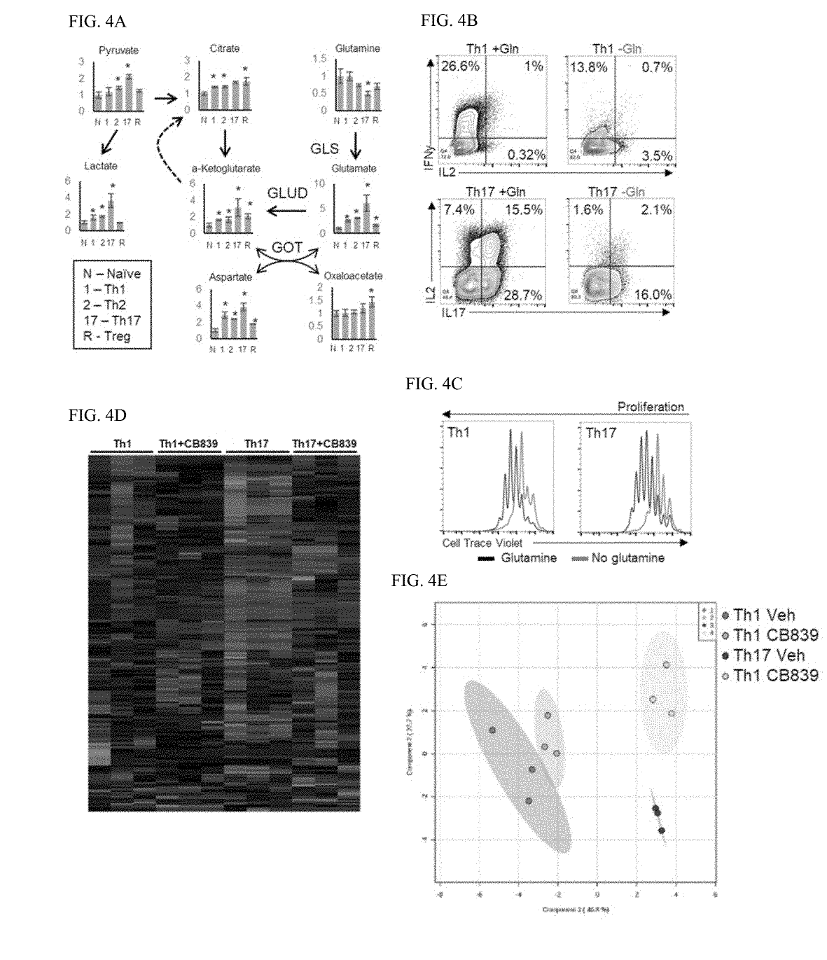

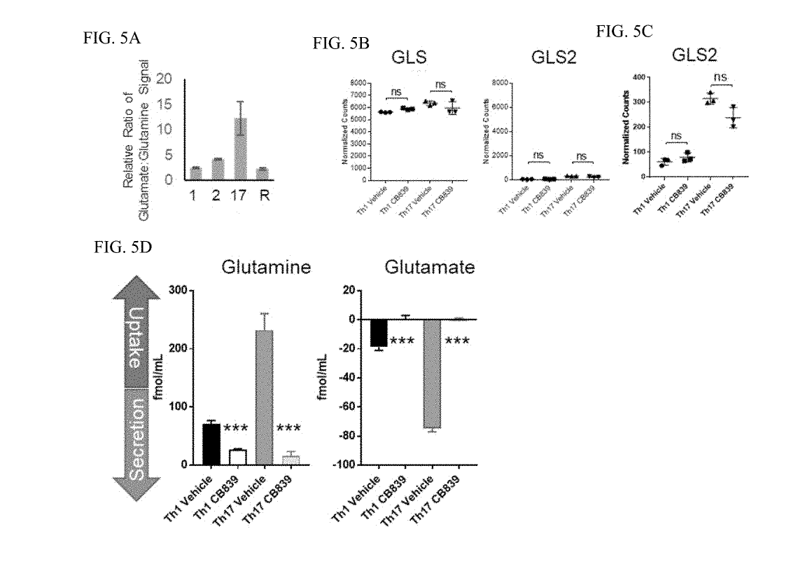

Distinct cytokines lead activated T cells to induce specific metabolic programs. To test if CD4 T cell subsets had different patterns of glutamine usage, metabolic data from in vitro differentiated Th1, Th2, Th17, and Treg cells were examined. T cells differentiated into Teff (Th1, Th2, and Th17) cells showed a strong increase in glutamate and α-KG. This increase was most pronounced in Th17 cells (FIG. 4A), where the ratio of the signal from glutamate to glutamine was highest (FIG. 5A). To test the role of glutamine in T cell subsets Th1 and Th17 cells were activated and differentiated in the presence or absence of glutamine. Both Th1 and Th17 required glutamine, as glutamine-deficiency markedly reduced Th1 production of interferon-gamma (IFNγ ) and Th17 production of IL-17 (FIG. 4B). Similarly, both Th1 and Th17 cells showed reduced proliferation in glutamine-deficient media (FIG. 4C).

Because both Th1 and Th17 cells required glutamine but had distinct profiles of glutamine, glutamate, and α-KG, the role of GLS in these subsets was examined. Th1 and Th17 cells were differentiated in vitro in the absence or presence of CB839 and subjected to metabolomics analyses. Th1 and Th17 cells had distinct metabolic profiles (FIGS. 4D, 4E). While programs of intracellular metabolites shifted in both Th1 and Th17 cells upon GLS-inhibition, this change was more pronounced in Th17 than in Th1 cells. GLS transcription remained unchanged (FIG. 5B). No compensation by GLS2 at the RNA level was found in CB839-treated Th1 or Th17 cells, and indeed GLS expression was almost 20-fold higher than GLS2 (FIG. 5C). Uptake and secretion of nutrients also showed distinctions between Th1 and Th17 cells, with higher basal glutamine uptake and glutamate secretion by Th17 that was dependent on GLS (FIG. 5D). Pathway analysis of metabolites that were altered upon CB839 treatment showed changes in key metabolic pathways, including alanine, aspartate, and glutamate metabolism. While Th17 cells were more strongly affected by GLS inhibition, fewer metabolic pathways were impacted than CB839-treated Th1 cells (Table 1). Thus, although both Th1 and Th17 cells require glutamine, GLS plays a differential role in the metabolisms of each.

TABLE 1

| | Total | Hits | Raw p | Hits Discovered |
|---|---|---|---|---|
| Th1 Pathway | | | | |
| Alanine, aspartate and | 24 | 8 | 5.34E−08 | Fumaric Acid; Pyruvic Acid; Ureidosuccininc Acid; L-Asperic Acid; |
| glutamate metabolism | | | | Argininosuccinic acid; L-glutamate acid; L-glutamine; Oxoglutamate acid |
| citrate cycle (TCA cycle) | 20 | 5 | 0.0001074 | Oxoglutamate acid; L-ratric acid; Pyruvic Acid; Fumaric acid; |
| | | | | Phosphoantpy [symbol] |
| D-Glutamine and D-glucamate | 5 | 3 | 0.000058541 | L-Glutamate acid; L-glutamine; oxoglutamate acid |
| metabolism | | | | |
| Pyrimidine metabolism | 41 | 6 | 0.00048197 | L-Glutamine; 4,5-Dihydroacidic acid; Dihydro [symbol] ; Cytidine monophosphate; |
| | | | | Cytidine; Ureidosuccininc acid |
| Arginine and proline metabolism | 44 | 6 | 0.00071444 | L-Glutamine; L-Aspartic acid; Arg [symbol] acid; L-Glutamine acid; |
| | | | | L-4-Hydro [symbol] [symbol] hyde; Fumaric acid |
| Histidine metabolism | 15 | 3 | 0.0060105 | L-Glutamine acid; [symbol] acid; L-aspartic acid |
| Butanoate metabolism | 22 | 3 | 0.007935 | Oxoglutamine acid; Pyruvic acid; 2-Hydroxy [symbol] |
| Pyruvate metabolism | 23 | 3 | 0.020258 | Pho [symbol] acid; Pyruvic acid; L- [symbol] acid; |
| Hitrogen metabolism | 9 | 2 | 0.021285 | L-Glucamate acid; L-Glutamine; |
| cysteine and methionine | 27 | 3 | 0.031146 | 5′-Methylthicadenosinte; 2-Aminoacrylic acid; Pyruvic acid; |
| metabolism | | | | |
| Th17 Pathway | | | | |
| Pentose phosphate pathway | 19 | 5 | 0.00433 | Deoxyribose 5-phosphate; D-Ribulose 5-phosphate; Sedohepholose |
| | | | | 7-phosphate; 6-Phosphoglutamaic acid; D-Erythrose 4-phosphate |
| Alanine, aspartate and | 24 | 5 | 0.01245 | Argininosuccinic acid; L-Alanine; Ureidosuccininc acid; |
| glutamate metabolism | | | | Succininc acid; Glucosamine G-phosphate |
| Phenylalanine, tyrosine and | 4 | 2 | 0.02017 | L-Phenylalamine; L-Tyrosine |
| tryptophan biosynthesis | | | | |
| Purine metabolism | 38 | 8 | 0.04882 | Xanthime; D-Ribulose 5-phosphate; ADP; Deoxyimosine; Hypoxanthine; |
| | | | | Guanosine triclorophosphate; Guanosine; Adenosine diphosphate ribose |

[symbol]

indicates data missing or illegible when filed

### GLS Deficiency Has Little Effect on Resting T Cells and Modulates Activation

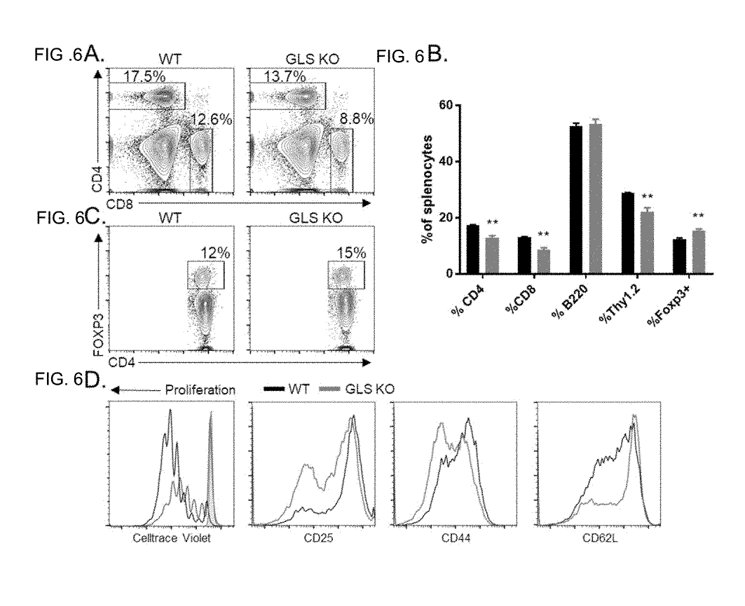

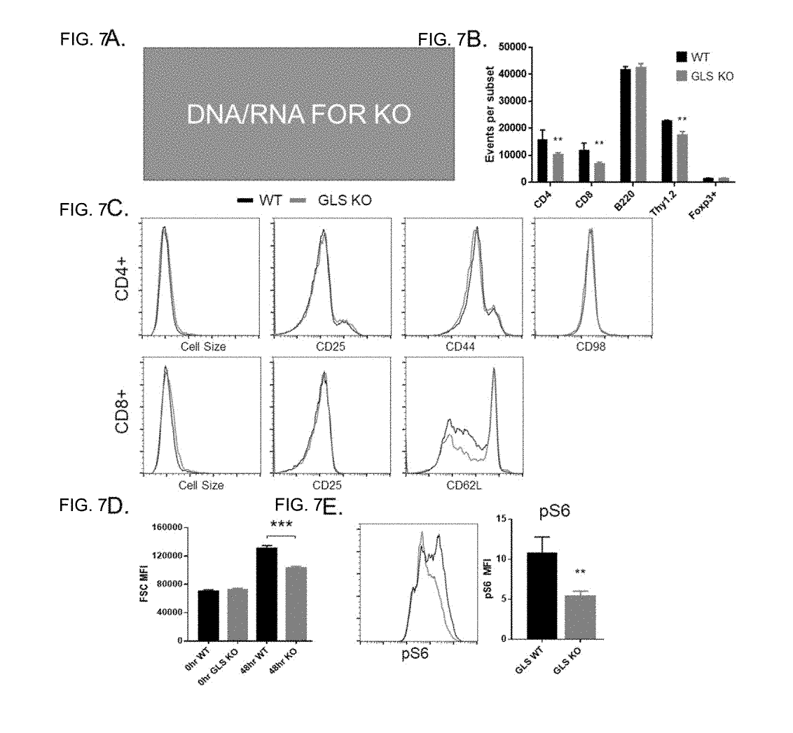

To further explore the role of GLS, a GLS^{fl/fl} model was generated and crossed to CD4-Cre to specifically delete Gls in T cells. Although GLS^{fl/fl} CD4-Cre T cells lacked expression of GLS (FIG. 6A), resting CD4 and CD8 T cell were only modestly reduced in frequency and number (FIG. 6A, FIG. 7B). Treg, in contrast, were modestly increased (FIGS. 6B, 6C, FIG. 7B), suggesting an independence of Treg from GLS. Despite decreased numbers, GLS^{fl/fl} CD4-Cre T cells had normal cell size and activation marker phenotypes (FIG. 7C). Upon activation, however, GLS-deficient T cells failed to efficiently undergo blastogenesis and increase in cell size (FIG. 7D), proliferate, induce CD25 and CD44, or downregulate CD62L (FIG. 6D). mTORC1 is a key regulator of T cell activation and anabolic metabolism and GLS^{fl/fl} CD4-Cre T cells had reduced phosphorylation of the mTORC1 downstream substrate, S6 (FIG. 7E). These results point to GLS1 as not essential in T cell homeostasis, but important for activation of effector T cells.

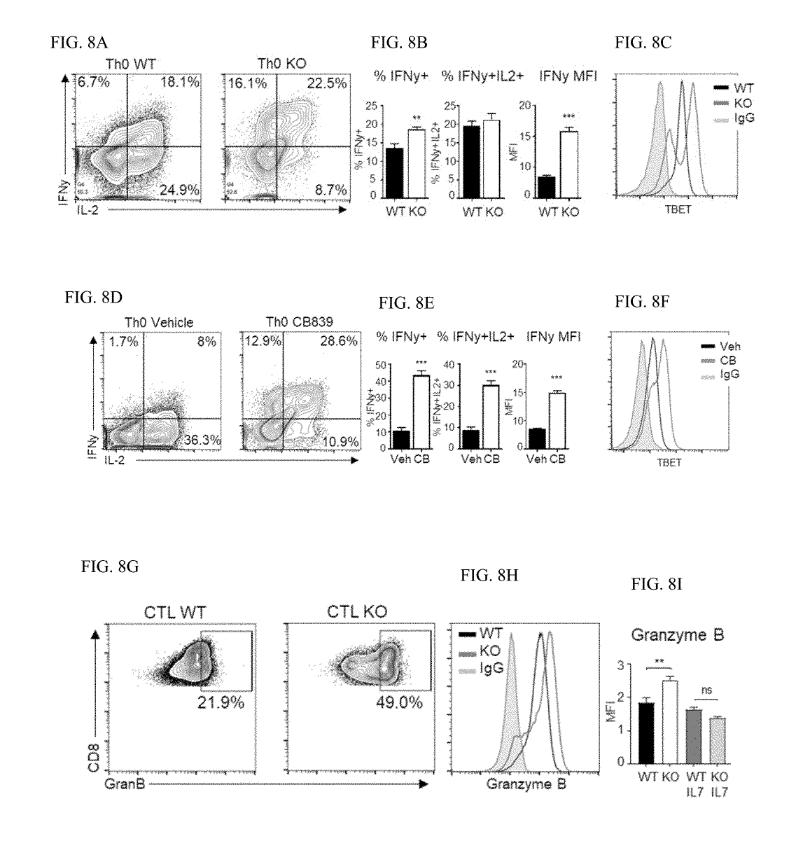

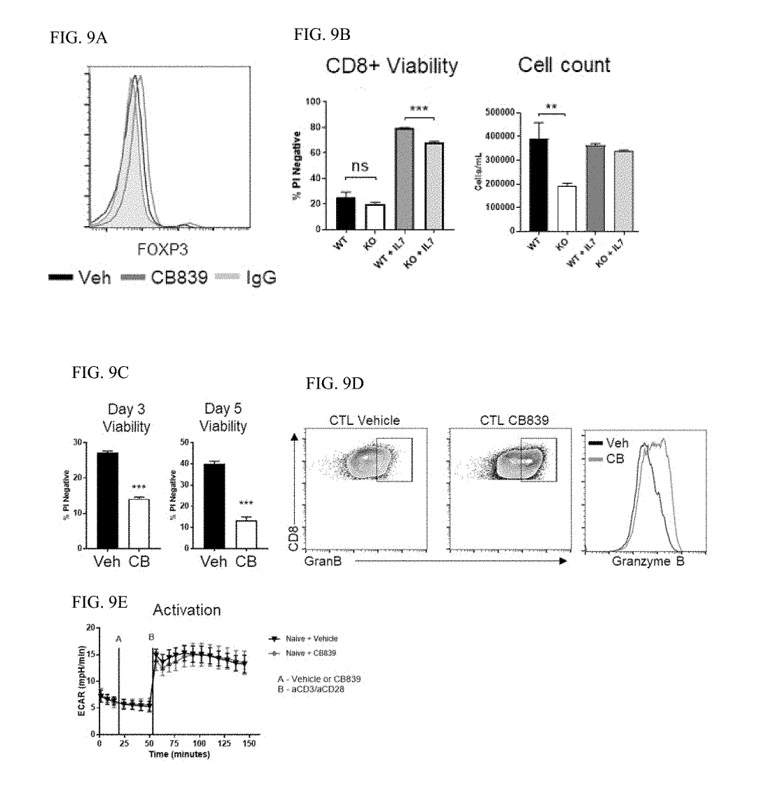

Decreased activation marker expression and proliferation in GLS-deficient T cells suggested impaired function and cytokine secretion. Control and GLS^{fl/fl} CD4Cre T cells were therefore activated and cultured in IL2 to examine cytokine production. Surprisingly, a greater frequency of activated GLS^{fl/fl} CD4-Cre T cells produced IFNγ than control T cells (FIGS. 8A, 8B). In addition, GLS-deficient cells that expressed IFNγ did so to a greater level than IFNγ-producing control T cells. IFNγ expression is regulated in part by the transcription factor, Tbet, which was also found to be elevated in GLS^{fl/fl} CD4-Cre T cells (FIG. 8C). The effects of CB839 on CD4 T cell cytokine production were next tested and yielded similar results, as GLS-inhibition led to greater expression of IFNγ (FIGS. 8D, 8E) and Tbet (FIG. 8F). Because glutamine withdrawal has been shown to promote Treg differentiation²³, CD4+ T cells were stimulated in the presence of IL-2 and CB839 to assess if T cells were preferentially becoming Treg. However, Foxp3 expression was unchanged (FIG. 9A).

Because Th1 and CD8+ (Cytotoxic Lymphocytes, CTLs) cells are both driven by Tbet³⁰, CD8 T cell induction of Granzyme B was assessed. Similar to GLS-deficient CD4 cells, GLS^{fl/fl} CD4-Cre CD8 T cells proliferated less well than controls. Although viability was unchanged, fewer GLS-deficient T cells accumulated upon stimulation (FIG. 9B). GLS^{fl/fl} CD4-Cre CD8 T cells had increased expression of the effector protein Granzyme B (FIGS. 8G, 8H, 8I). Similarly, CD8 T cells treated with GLS inhibitor also had increased levels of Granzyme B (FIG. 9D). Early events in T cell activation, however, did not to be affected by CB839 (FIG. 9E). Together, these data show that GLS-deficiency in CD4 or CD8 cells does not interfere with initial events, but ultimately decreases activation while promoting expression of effector programs.

### GLS Plays Differential Roles in T Cell Effector Subsets

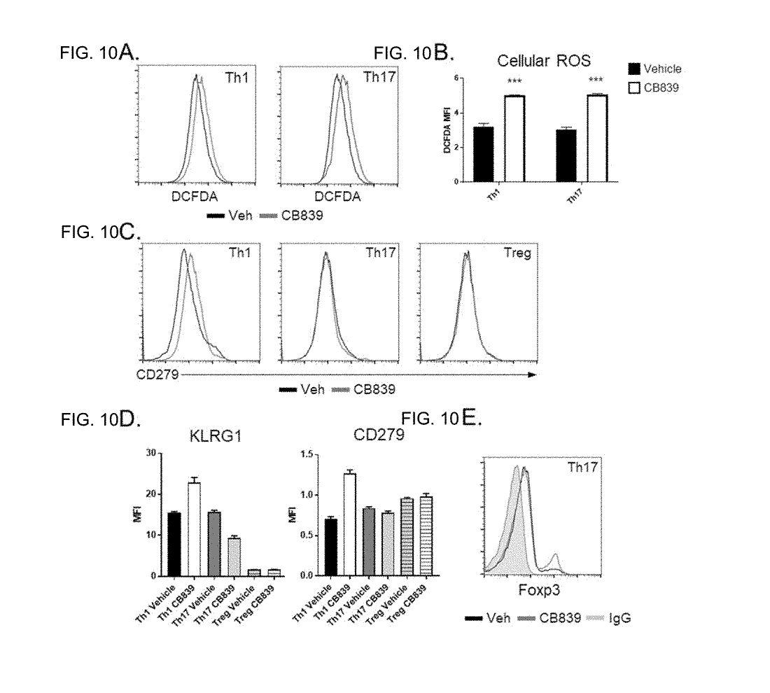

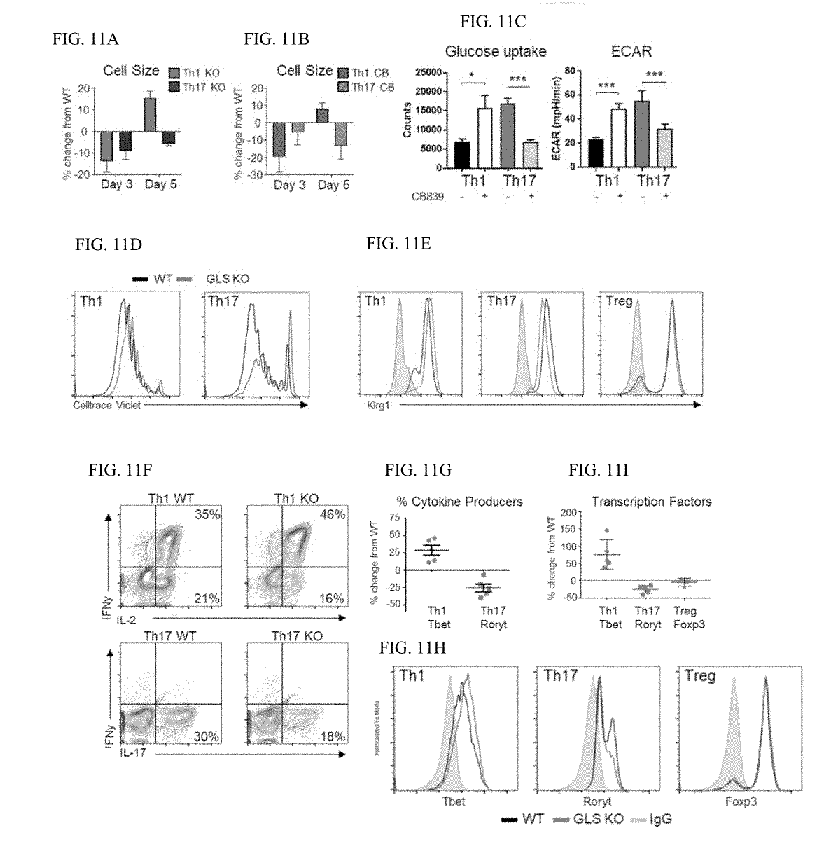

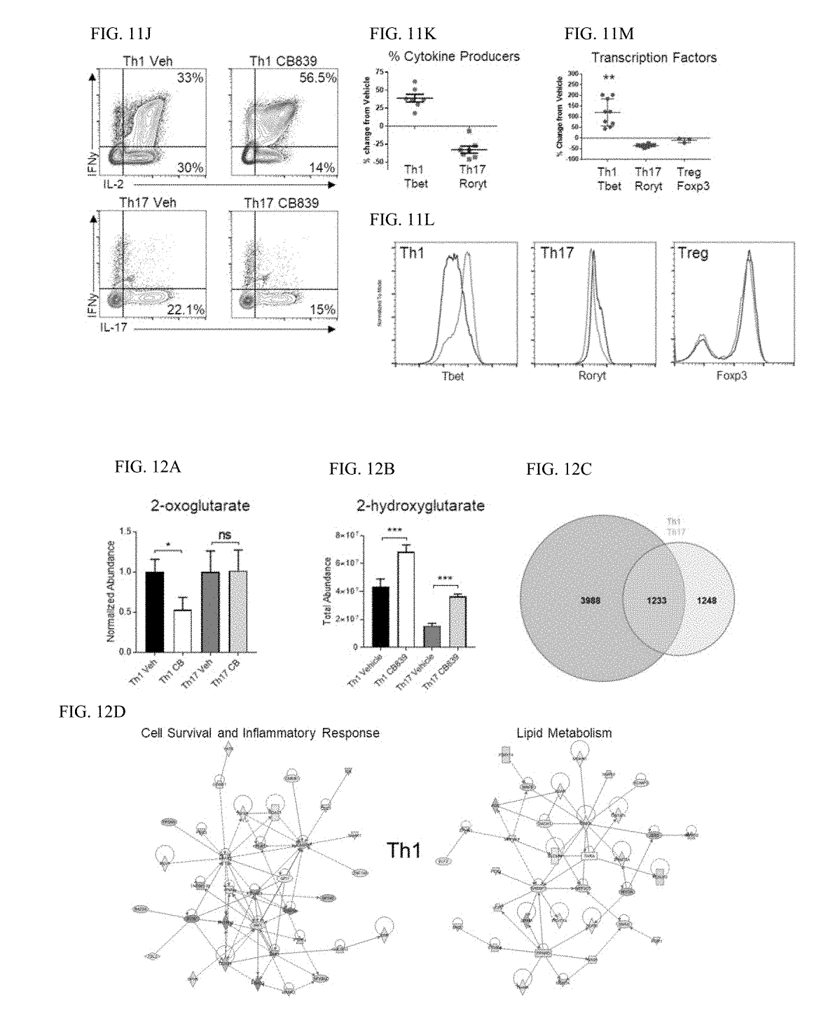

Because GLS null or inhibited T cells showed increased effector functions upon stimulation, it was possible that GLS-deficiency affected T cell differentiation. To test this, control and GLS^{fl/fl} CD4-Cre or GLS-inhibited CD4 T cells were differentiated in vitro into Th1, Th17, and Treg subsets. GLS may contribute to cellular redox regulation through generation of glutamate for glutathione synthesis and both Th1 and Th17 cells were found to have increased ROS when treated with CD839 (FIG. 10A, B). However, while GLS-deficient T cells in Th1 and Th17 conditions each showed an initial smaller gain in cell size than activated control T cells, Th1 and Th17 cells diverged at later time points and Th1 cells were larger and Th17 cells were remained smaller than controls (FIGS. 11A, 11B). Consistent with these findings and different metabolic responses of Th1 and Th17 to GLS inhibition (FIG. 4D), Th1 and Th17 cells treated with GLS inhibitor had opposite responses in key measurements of glucose metabolism (FIG. 11C). CB839 increased glucose uptake and media acidification on day 5 in Th1 cells, while these were decreased in Th17 cells. Also, while T cell proliferation was suppressed in both Th1 and Th17, GLS-deficient Th17 cells were more strongly affected (FIG. 11D). Th1 and Th17 cells also appeared to differentiate differently, as CB839-treated Th1 cells had increased expression of KLRG1 and PD1 while these markers were decreased or unchanged in Th17 (FIG. 11E, FIGS. 10C, 10D).

The ability of Th1, Th17, and Treg to produce effector cytokines and differentiate was next directly assessed. Similar to T cell activated in only IL2 (FIG. 8), a greater percentage of GLS^{fl/fl} CD4-Cre T cells expressed IFNγ when differentiated in Th1 conditions (FIGS. 11F, 11G). A decreased percentage of GLS-deficient T cells expressed IL17 when stimulated in Th17 conditions. Expression of effector molecules and differentiation in Th1, Th17, and Treg are regulated by Tbet, RORγt, and FoxP3, respectively. Consistent with cytokine expression, GLS-deficient T cells showed increased Tbet under Th1 conditions and decreased RORγt under Th17 conditions (FIGS. 11H, 11I). In contrast, FoxP3 expression was unchanged in the absence of GLS. Similar results were obtained when GLS was acutely inhibited with CB839, as Th1, Th17, and Treg differentiation were increased, decreased, or unchanged, respectively (FIG. 11J-M). While possible that GLS-deficient Th17 cells that failed to express RORγt and IL17 instead differentiated into an alternate subset, neither IFNγ nor FoxP3 were significantly elevated in GLS-deficient T cells stimulated in Th17 conditions (FIGS. 11F, 11J, FIG. 10D).

### GLS Affects Gene Expression and Chromatin Accessibility

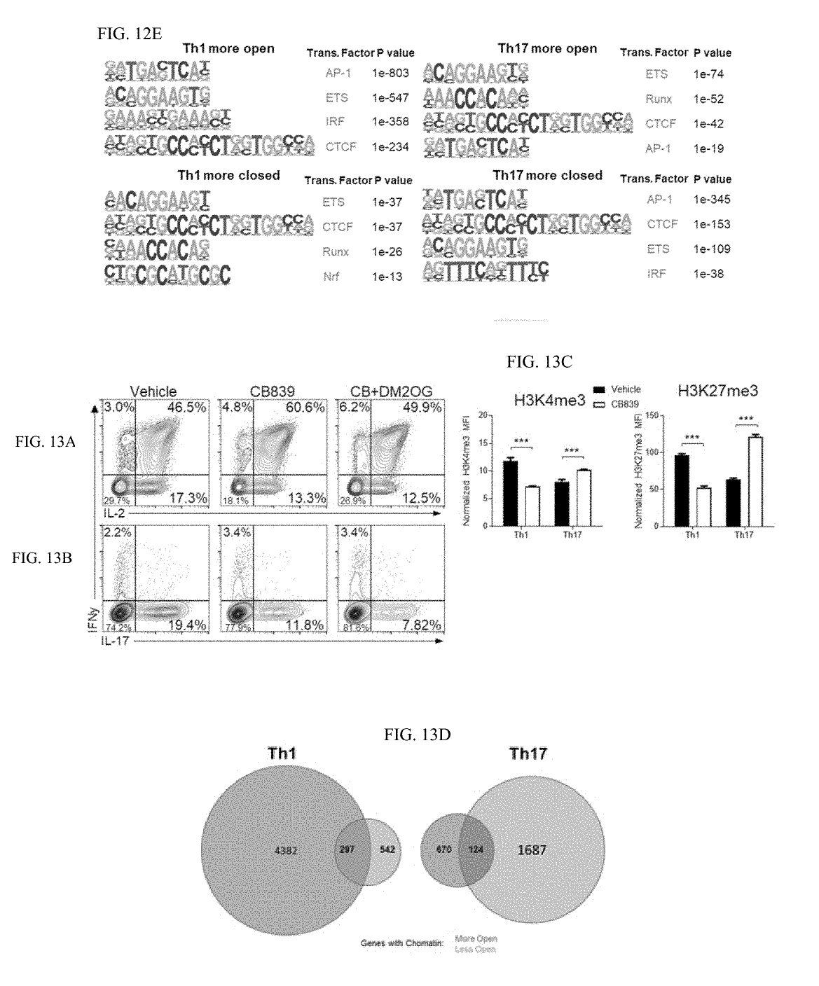

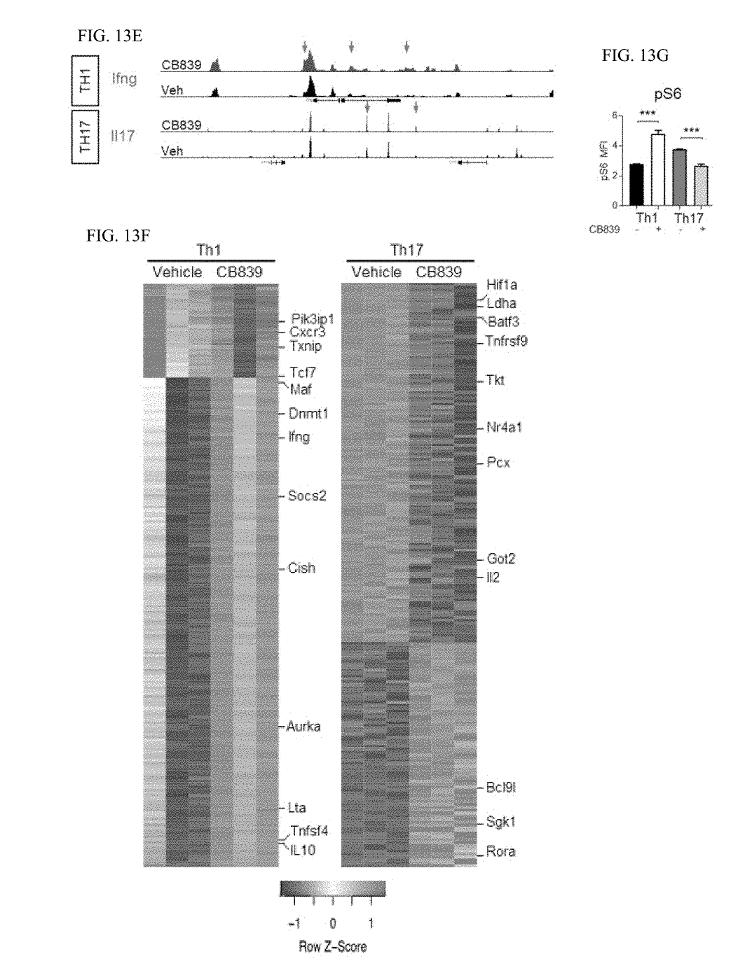

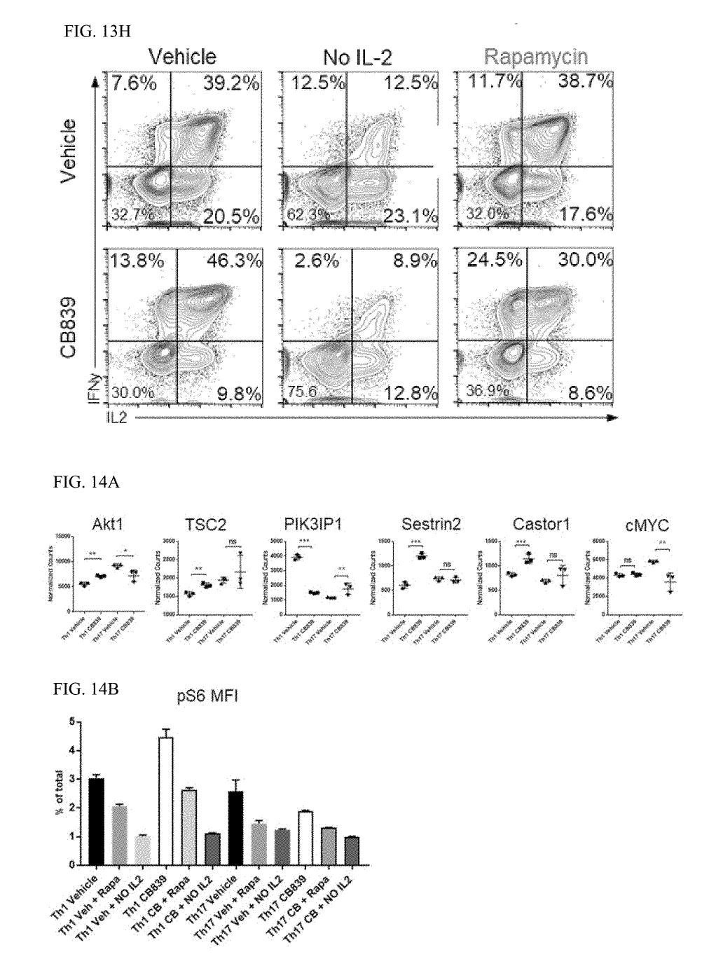

The opposing effects of GLS deficiency on differentiation of Th1 and Th17 cells suggested altered gene expression and epigenetic regulation. Deficient GLS activity may lead to changes in gene expression through production of substrates for epigenetic marks and changes in chromatin status. GLS can affect α-KG and 2-hydroxyglutarate, which can promote or inhibit demethylation reactions (Xu et al. 2017 Nature 548(7666):228-233). Based on intracellular metabolite analysis by mass spec, α-KG was reduced in CB839-treated Th1, but not Th17 cells (FIG. 12A). 2-hydroxyglutarate (2-HG), however, increased in both Th1 and Th17 (FIG. 12B). The reduced α-KG in CB839-treated Th1 cells suggested that α-KG may become limiting to regulate Th1 differentiation and function. A cell-permeable α-KG analog, dimethyl-2-oxoglutarate (DM2OG), was therefore tested to determine if provision of α-KG could restore normal differentiation of CB839-treated T cells. Consistent with limiting α-KG leading to altered differentiation in Th1 cells, DM2OG reduced IFNγ production of CB839-treated to control levels (FIG. 13A). Consistent with maintenance of α-KG levels, Th17 cells were not rescued and IL17 production was unchanged or further decreased by DM2OG (FIG. 13B), suggesting a distinct mechanism of regulation for Th17 cells by GLS.

Changes in α-KG and 2-HG may lead to changes in histone methylation and chromatin accessibility that influence T cell differentiation (Xu et al. 2017 Nature 548(7666):228-233). Tri-methylation of Histone H3 K4 and K27 was assessed globally by flow cytometry. When normalized for total accessible Histone H3, CB839-treated Th1 and Th17 cells were found to have decreased or increased global H3K4 and H3K27 trimethylation, respectively (FIG. 13C). Because, in principle, multiple epigenetic marks were altered by GLS-deficiency, an Assay for Transposase-Accessible Chromatin sequencing (ATACseq) was performed to determine if GLS regulated chromatin accessibility in Th1 and Th17 cells. Similar to the increased gene expression in CB839-treated Th1 cells, Th1 cells had more genes with regions of increased accessibility than genes with decreased accessibility (FIG. 13D). Conversely, Th17 cells had more genes with regions of reduced accessibility. While partially overlapping, affected genes were largely distinct for Th1 and Th17 cells (FIG. 12C). Key Th1 and Th17 genes showed changes, including the IFNγ and IL17A/F loci in Th1 and Th17 cells, respectively (FIGS. 13E, 12D). Further, Ingenuity Pathway analyses of genes with altered accessibility in the promoter regions of Th1 cells showed changes in networks of cell survival and inflammation as well as lipid metabolism (FIG. 12D). Analysis of promoter regions with altered accessibility identified recognition motifs for canonical T cell differentiation transcription factors, such as AP-1, ETS, and IRF (FIG. 12E). These altered promoter regions were also enriched in CTCF recognition motifs.

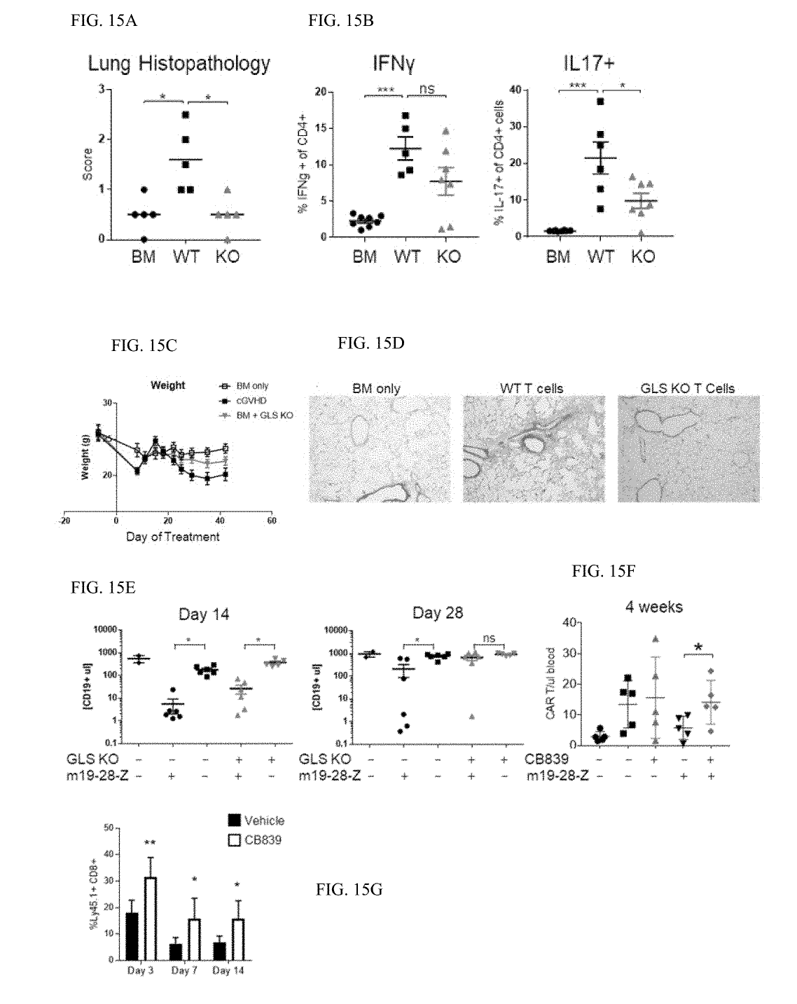

Because altered chromatin accessibility can influence gene expression and T cell differentiation, T cells were cultured in Th1 or Th17 conditions with vehicle or CB839 and examined by RNA sequencing (FIG. 13F). Interestingly, of the 200 genes with the most altered expression in CB839-treated Th1 cells, the majority showed increased expression. Conversely, more of the most changed genes were downregulated in Th17 cells. Gene set enrichment pathway analyses showed that GLS-inhibition led to upregulation of cell cycle, mTORC1, Myc, IL2 signaling, and glycolysis pathways in Th1 (Table 1). Conversely, these gene sets were downregulated in CB839-treated Th17 cells. Indeed, components of the mTORC1 pathway, Pik3ipl, Akt, Tsc2, Sestrin2, and Castor1, were specifically regulated in Th1 cells consistent with increased potential for PI3K/Akt/mTORC1 signaling (FIGS. 14A, 14B).

Signaling through mTORC1 may be altered in Th1 and Th17 cells and contribute to increased Th1 effector function. Levels of the mTORC1 downstream target phosphor-S6 were measured in Th1 and Th17 cells differentiated in the presence or absence of CB839 to determine if mTORC1 activity was altered. Consistent with differential regulation of mTORC1 regulation, GLS-deficiency led to increased phosphor-S6 in Th1 and decreased phosphor-S6 in Th17 cells (FIG. 13G). The IL2 signaling pathway can activate mTORC1 and was increased in Th1 by RNAseq gene set enrichment analysis (Table 1) that drove mTORC1 signaling in GLS-deficient T cells (FIG. 14C). Indeed, culture of Th1 cells without IL2 sharply decreased cytokine production in both control and CB839-treated cultures (FIG. 13H). The role of mTORC1 signaling in GLS-mediated regulation of Th1 cells was directly tested by treatment of cells on day 3 after activation with the mTORC1 inhibitor, rapamycin. While rapamycin treatment at this time had no effect on control Th1 cells, it reduced cytokine production in CB839-treated Th1 cells (FIG. 13H). These data support a model in which GLS-deficiency leads to altered chromatin and gene expression that enhance sensitivity of Th1 cells to IL2 and mTORC1 signaling.

### Example 2

### Glutaminolysis and Graft-vs-Host Disease (cGvHD)

Th17 and Th1 cells were differentially regulated by GLS-deficiency in vitro. Nutrient conditions and regulation, however, differ in vivo and the role of GLS may differ. A model of IL17-dependent chronic Graft-vs-Host Disease (cGvHD) was used to test the dependence of Th17 cells on GLS.

### Materials and Methods

### Mice

Mice were obtained from the Jackson laboratory or as described elsewhere (Young et al., 2011 PLoS One 6(8):e23205). GLS^{fl/fl} animals were obtained as embryonic stem cells from the KOMP and crossed to FLP transgenic animals to delete the Neo cassette. These progeny were then crossed with CD4-CRE transgenic mice to develop the GLS^{fl/fl} CD4-CRE (GLS KO). In all cases comparing wild type to GLS KO, sex-matched and age-matched littermates were used. All procedures were performed under appropriate IACUC-approved protocols.

### In Vivo Graft Versus Host Disease

Induction of Graft vs Host Disease (cGVHD) was performed as described elsewhere (see, e.g., Panoskaltsis-Mortari et al., 2007 Am J Respir Crit Care Med. 176(7):713-723). Briefly, mice were lethally irradiated the day before bone marrow (BM) transplant. Mice were dosed with cyclophosphamide (Cytoxan, Bristol Myers Squibb, Seattle Wash.) at 120 mg/kg/day on days −3 and −2. Recipient irradiated mice were transplanted via caudal vein with 10×10⁶ T-cell depleted allogeneic marrow with 73.5×10³ purified splenic T cells from WT or GLS KO mice, or control (no CD4+ T cells). Mice were assessed for lung elasticity, resistance, and compliance at Day 49 by whole body plethysmography using the Flexivent system (Scireq, Montreal, PQ, Canada). Histological assessment of GVHD was assessed as described elsewhere (see, e.g., Blazar et al., 1998 Blood 92(10):3949-3959).

### In Vivo CAR T Cells

CAR T cells were produced as described elsewhere (see, e.g., Li et al., “Gammaretroviral Production and T Cell Transduction to Genetically Retarget Primary T Cells Against Cancer.” In: Lugli E, ed. T-Cell Differentiation: Methods and Protocols. New York, N.Y.: Springer New York; 2017:111-118). Briefly, spleen T cells were isolated from Thy1.1 B6 mice at day 0. Cells were then activated with mouse CD3/CD28 Dynabeads and 30 IU/ml recombinant human IL2. At day 1 and 2, cells were spin transduced twice with retrovirus carrying CARs. At day 3, cells were fed with fresh medium. At day 4, transduced T cells were harvested, beads removed, evaluated for viability, transduction efficiency, immune phenotype and ready for use. For CB839 treated CAR T cells, CB839 were added to the culture at day 1, 2 and 3 at 1 μM. For in vivo study, C57B6 mice (n=25) were i.p. injected with cyclophosphamide (CTX) at 300 mg/kg. Mice were i.v. injected with 3×10⁵ CAR T cells one day after CTX injection. Peripheral blood (PB) samples were collected 1, 2, 4 and 6 weeks after CAR T injection, stained with B cell and T cell antibodies and subjected to flow cytometry. CountBright beads were added to measure B and T cell numbers.

### Statistical Analysis

Statistical analyses were performed with Prism software (GraphPad) using the student T-test, one-way ANOVA unless otherwise noted. Longitudinal data was analyzed by two-way ANOVA followed by Tukey's test and followed up with one-way ANOVA or T-test as specified. Statistically significant results are indicated (*p<0.05) and n.s. indicates select non-significant data. Error bars show mean±Standard Deviation unless otherwise indicated. RNA-Seq data were analyzed by DESeq2 in R.

### Results

### GLS is Essential In Vivo For Inflammatory Effector T Cell Responses

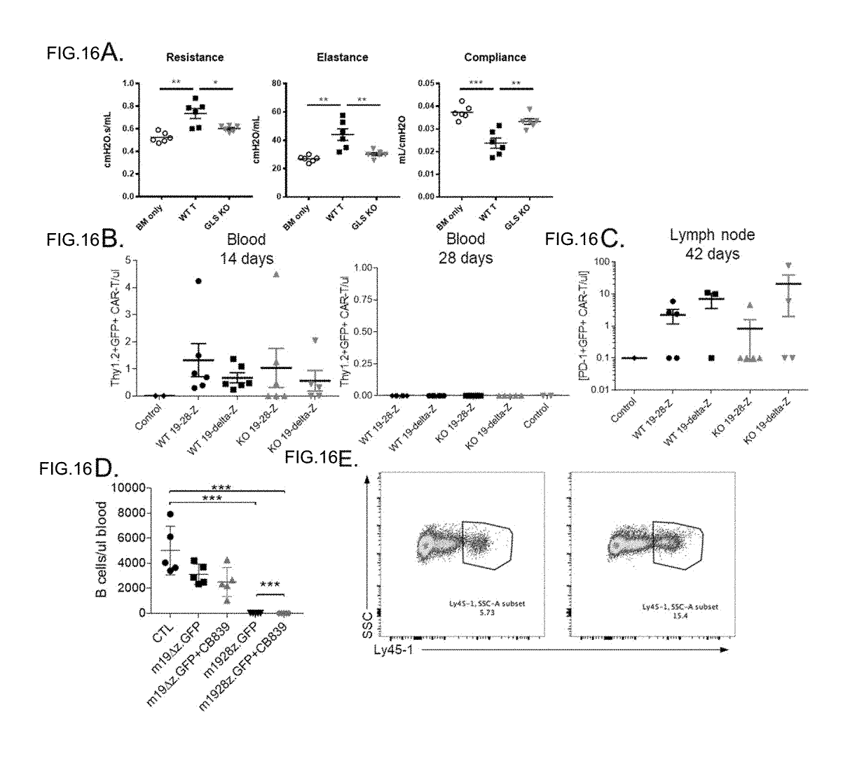

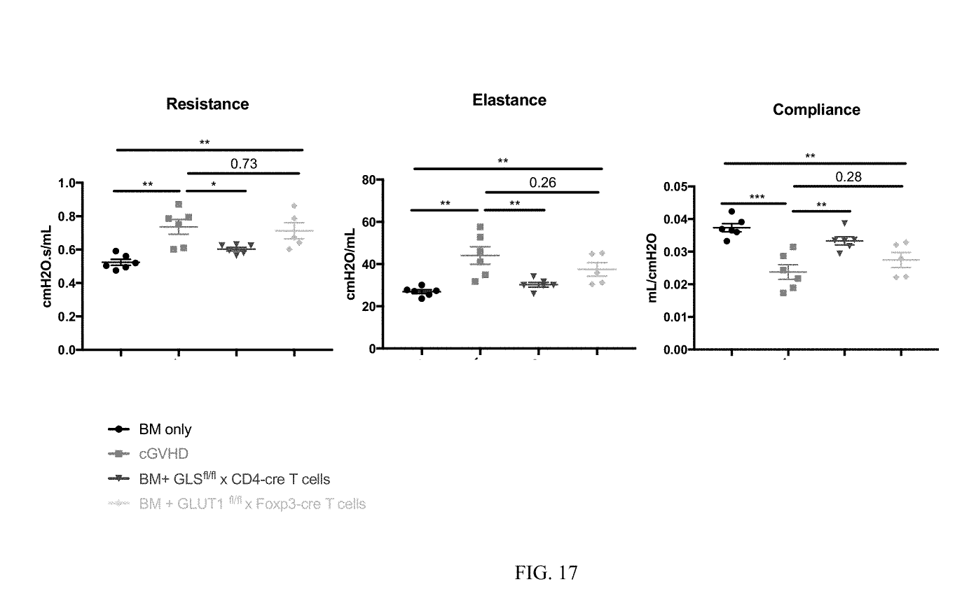

Allogenic bone marrow was transplanted alone or with control and GLS^{fl/fl} CD4-Cre T cells to induce cGvHD. Recipient mice were weighed regularly and GLS-deficient allogenic T cells were found to induced less weight loss than control T cells (FIG. 15A). Inflammation in cGvHD is multi-focal and includes lung. Consistent with reduced ability of GLS-deficient T cells to induce cGvHD, GLS-deficient T cells caused significantly less airway functional impairment than control T cells (FIG. 16A). Further, histological examination of lungs showed GLS-deficient T cells led to reduced immune infiltrate and lower clinical inflammation scores (FIGS. 15B, 15C). Immunologically, CD4 T cells from GLS KO had reduced numbers of IL-17 and IFNγ producing cells, indicating an in vivo deficit to produce inflammatory cytokines (FIG. 15D).

The role of GLS to increased Th1 and CTL differentiation and function was next tested in vivo. Control and GLS^{fl/fl}CD4-Cre T cells were first tested in a Chimeric Antigen Receptor (CAR) model for ability to eliminate B cell targets and persist in vivo. T cells were in vitro transduced with CAR-T expression vectors either lacking a cytoplasmic tail or with a CD3ζ-D28 intracellular tail and adoptively transferred. 14 days after T cell transfer CD19 expressing targets were significantly reduced by both control and GLS-deficient CAR-T cells (FIG. 15E). After 28 days, however, CD19 expressing targets had accumulated in recipients of GLS-deficient CAR-T cells. Thus, although Th1 and CD8 differentiation were increased with GLS-deficiency and CAR-T cells were initially functional, T cells appeared unable to sustain an effector response in vivo in the absence of GLS activity. Because GLS-inhibition could alter Th1 chromatin accessibility, however, it was possible that transient treatment with CB839 may induce long lasting effect. T cells were treated with vehicle or CB839 during in vitro transduction to express CAR and tested for subsequent in vivo function. Untreated and in vitro CB839-treated CAR-T cells were equivalently capable of eliminating CD19⁺ targets in vivo (FIG. 16D). However, in vitro CB839-treated m19-28-Z CAR-T cells accumulated in vivo to a greater extent than control m19-28-Z CAR-T cells (FIG. 15F). This increased ability of Th1 and CD8 effector T cells to proliferate or persist following transient GLS inhibition was not specific to CAR-T cells. CD8 T cells bearing a Pmel-specific TCR transgene treated with CB839 in vitro prior to adoptive transfer also accumulated to greater numbers when challenged with a Pmel-expressing vaccinia virus (FIG. 15G). Thus, chronic or complete GLS deficiency impairs T cell responses in vivo, while transient inhibition may reprogram Th1 and CD8 CTL to enhance effector function and cell numbers in vivo.

### GLS Deficiency Improves Pulmonary Function

Mice that received control and GLS^{fl/fl} CD4-Cre T cells were assessed for lung elasticity, resistance, and compliance. GLS deficiency improved pulmonary function in the mice by decreasing resistance, decreasing elastance, and increasing compliance (FIG. 17).

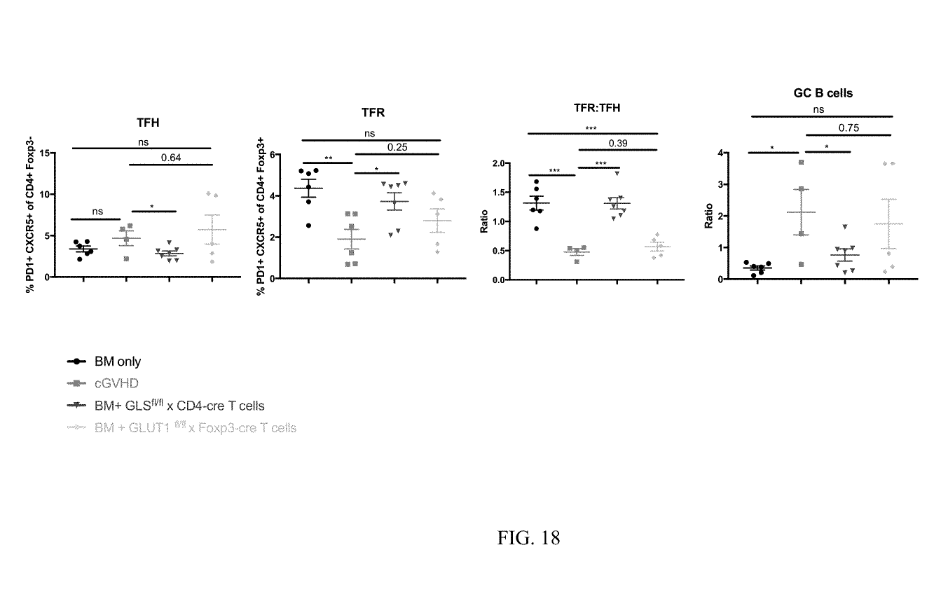

T cell subsets and B cells were identified and quantified. GLS deficiency alters lymphocyte numbers and percentages by decreasing T_{FH} and GC B cell frequencies and improving T_{FR}:T_{FH} ratios (FIG. 18).

### Example 3

### Distinct Regulation of Th17 and Th1 Cell Differentiation by Glutaminase-Dependent Metabolism

Activated T cells differentiate into functional subsets with distinct metabolic programs. Glutaminase (GLS) converts glutamine to glutamate to support the tricarboxylic acid cycle and redox and epigenetic reactions. This example identifies a key role for GLS in T cell activation and specification. Though GLS-deficiency diminished initial T cell activation, proliferation and impaired differentiation of Th17 cells, loss of GLS also increased Tbet to promote differentiation and effector function of CD4 Th1 and CD8 CTL cells.

### Results

### GLS and Glutaminolysis Contribute to T Cell Metabolism Upon Activation

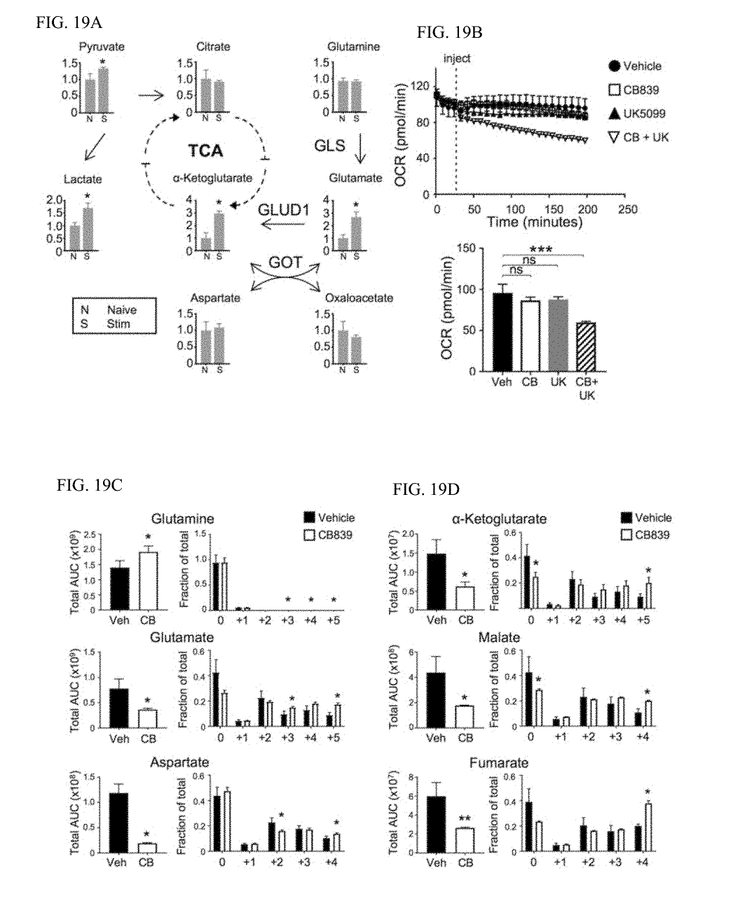

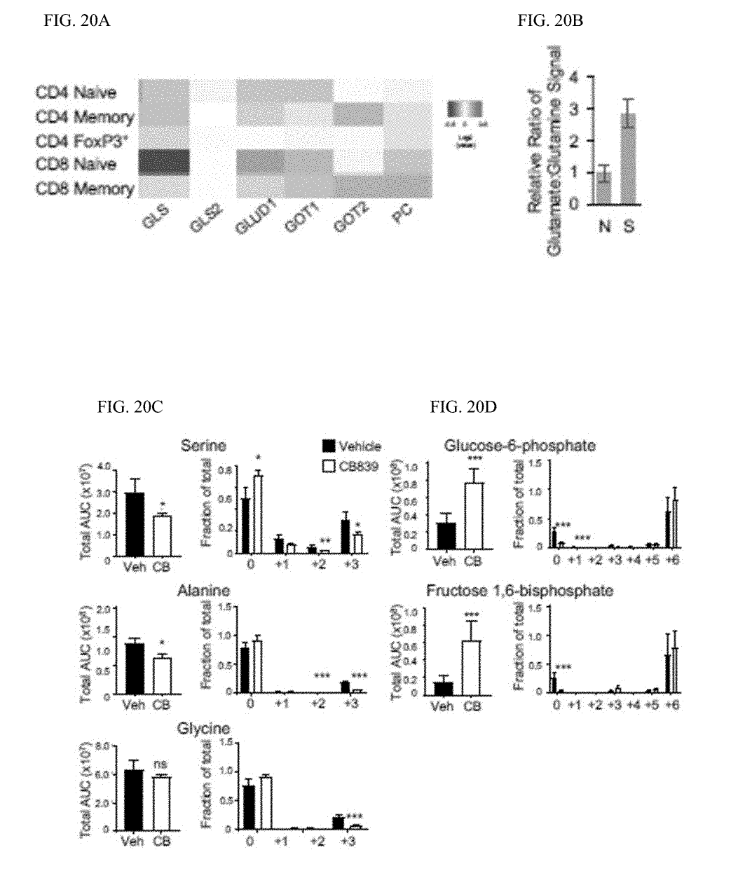

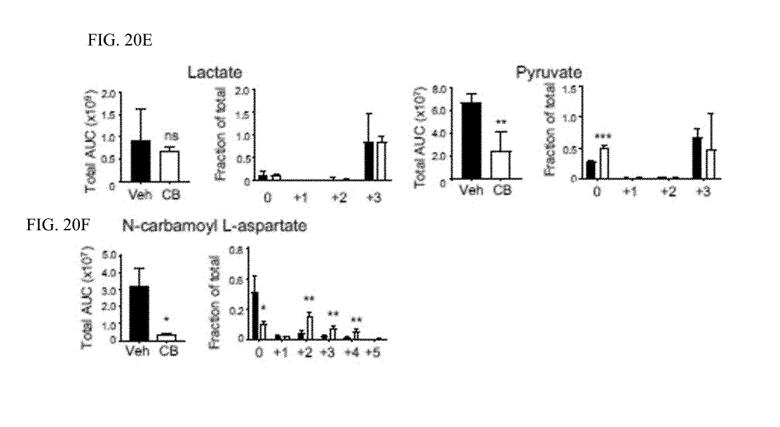

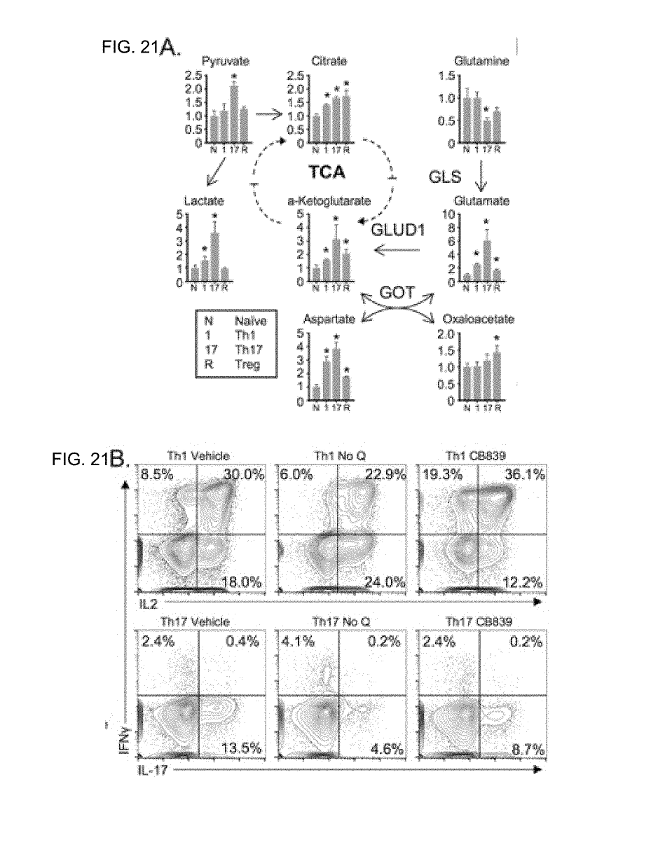

Activated T cells have significant metabolic requirements to support proliferation and differentiation. To determine the relative roles of glucose and glutamine in these processes, intracellular metabolites were measured following activation of CD4 T cells. In addition to increased pyruvate and lactate, glutamate and α-KG levels increased, suggesting elevated glutamine metabolism (FIG. 19A). Intracellular glutamate is primarily generated from glutamine by GLS or from α-KG and aspartate by GOT1 and GOT2 and is converted to α-KG by Glutamate Dehydrogenase 1 (GLUD1), which are each expressed in CD4 and CD8 T cells (FIG. 20A). The increased levels of both α-KG, glutamate, and high relative ratio of glutamine to glutamate (FIG. 20B), suggested GLS as a key source of glutamate and α-KG. To determine the relative roles of glutaminolysis and glycolysis as fuels for mitochondrial metabolism, oxygen consumption of stimulated T cells treated with mitochondrial pyruvate carrier (UK5099) or GLS (CB839) inhibitors was measured. While neither UK5099 nor CB839 were sufficient to reduce T cell respiration alone, the combination led to a significant decrease in oxygen consumption (FIG. 19B). These data demonstrate that stimulated T cells utilize glycolysis and GLS-dependent glutaminolysis.

To directly determine how inhibition of GLS affects glucose metabolism, CD4 T cells were stimulated in uniformly labeled ¹³C-glucose with or without CB839 and glucose derived carbons were traced. As expected, inhibition of GLS led to increased intracellular glutamine and decreased glutamate (FIG. 19C). Aspartate levels also decreased significantly. Glucose-derived ¹³C was increased in both glutamate and aspartate, indicating a greater fraction of glucose contribution to synthesis of these amino acids. Serine and alanine overall abundance also decreased, while glycine was unchanged and each showed a decreased portion derived from glucose (FIG. 20C). Overall levels of TCA intermediates were also reduced, yet with increased fractional labeling from glucose-derived ¹³C (FIG. 19D). Glycolytic intermediates were more abundant upon GLS-inhibition, suggesting elevated glycolysis (FIG. 20D). However, lactate levels and ¹³C-labeling were unchanged and pyruvate levels decreased (FIG. 20E). Anabolic pathways were also affected, and total levels of the nucleotide precursor N-carbamoyl-aspartate decreased (FIG. 20F). Thus, glucose metabolism increased and a greater fraction of glucose carbon entered the TCA cycle when GLS was impaired.

### CD4 T Cell Subsets Have Distinct Programs of Glutamine Metabolism

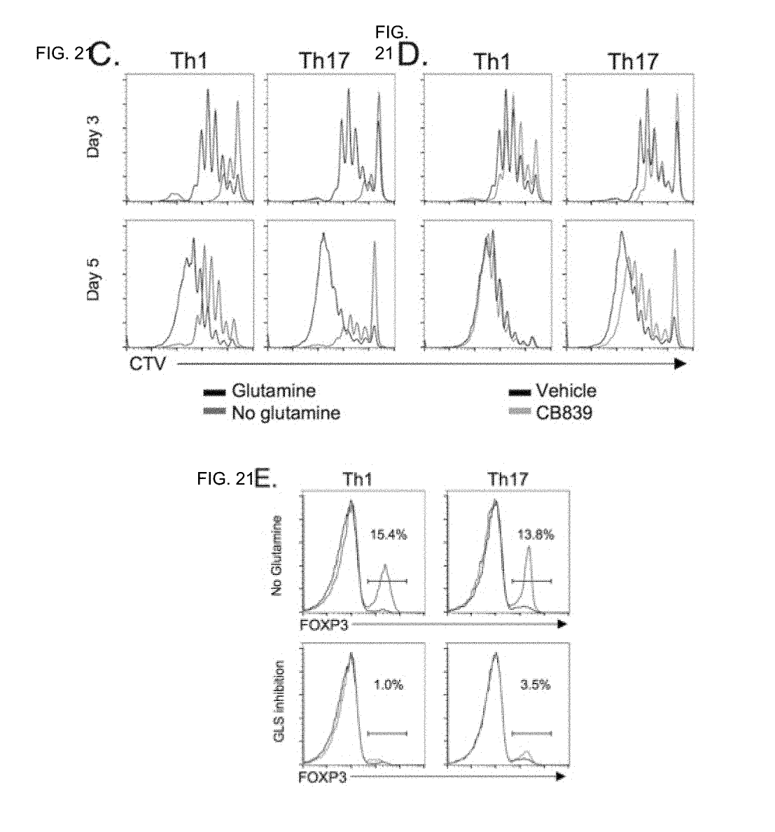

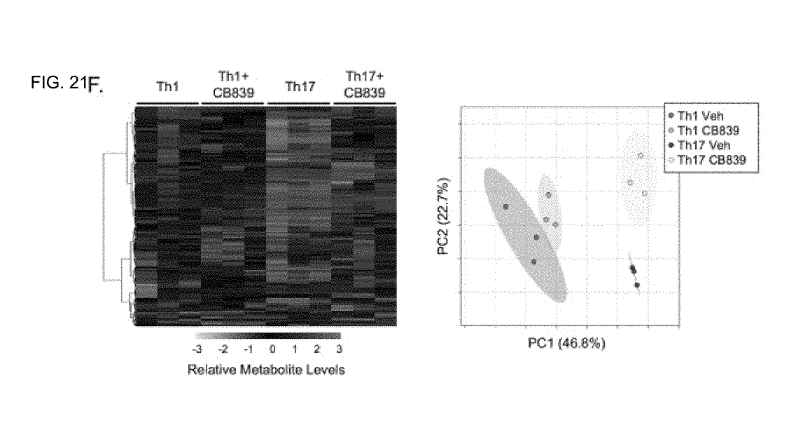

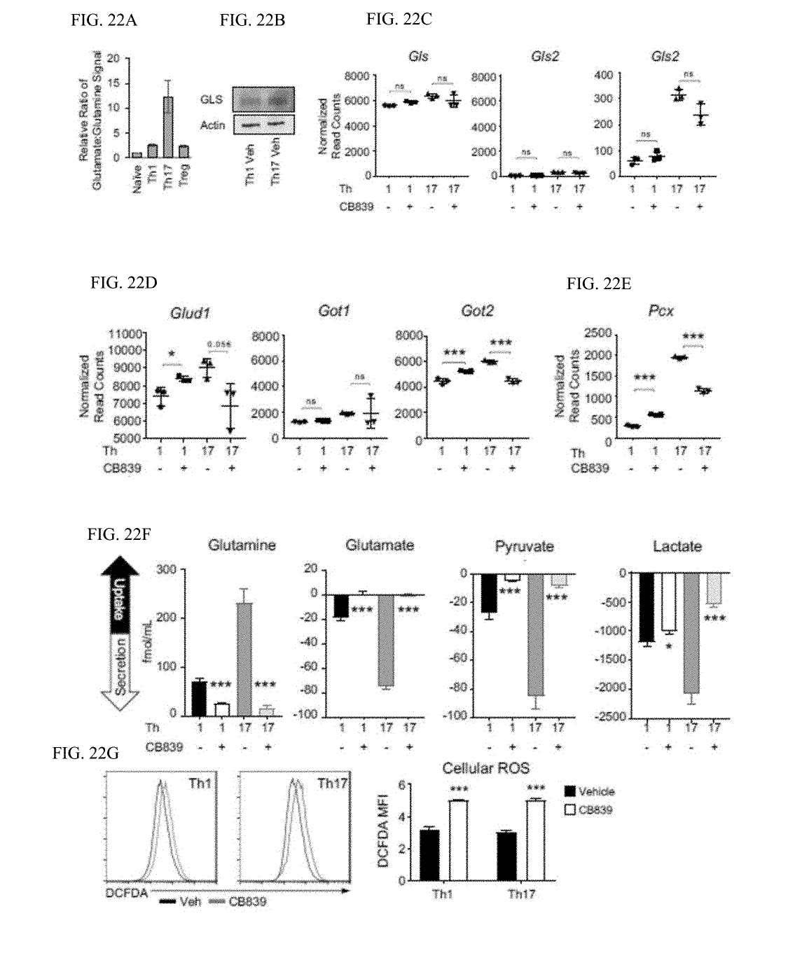

Distinct cytokines lead activated T cells to induce specific metabolic programs. Th1, Th17, and Treg cells were examined to assess if CD4 T cell subsets had different patterns and reliance on glutamine metabolism. T cells activated and differentiated into each subset showed increased glutamate and α-KG levels relative to naïve T cells. This was most pronounced in Th17 cells (FIG. 21A), which also had the highest relative ratio of glutamate to glutamine (FIG. 22A). To test the role of glutamine and GLS in Th1, Th17, and Treg T cell subsets, CD4 T cells were differentiated with or without glutamine, or with GLS inhibitor. Both Th1 and Th17 required glutamine, as glutamine-deficient media markedly reduced Th1 production of IFNγ and Th17 production of IL-17, yet GLS-inhibition decreased cytokine production and proliferation only from Th17 cells and appeared to increase Th1 cytokine secretion (FIG. 21B). Glutamine deficiency reduced proliferation at day three and five in both Th1 and Th17 cells. GLS-inhibition impaired proliferation of both Th1 and Th17 cells after three days in culture (FIG. 21C, D). CB839-treated Th1 cells partially recovered proliferation by day five. Glutamine deprivation also induced Treg even under Th1 and Th17 conditions, yet GLS inhibition failed to do so (FIG. 21E). GLS-deficiency, therefore, has distinct effects on T cell subsets from glutamine deprivation. Several enzymes contribute to regulation of glutaminolysis in T cells. Th17 cells had greater expression of GLS protein than Th1 at protein and RNA levels (FIG. 22B, C). Th1 and Th17 cells expressed low levels of Gls2 mRNA and expressed similar levels of other glutamine and anaplerotic metabolic enzymes (FIGS. 22C-E). Th1 and Th17 cells had distinct metabolic profiles and intracellular metabolites shifted in both Th1 and Th17 cells upon GLS-inhibition, including alanine, aspartate, and glutamate metabolism pathways (FIG. 21F and Table 2). Nutrient uptake and secretion also differed between Th1 and Th17 cells and were modified by GLS inhibition. Glutamine uptake and glutamate, pyruvate, and lactate secretion were higher in Th17 but reduced upon GLS inhibition (FIG. 22F). GLS may contribute to cellular redox regulation through generation of glutamate for glutathione synthesis and ROS increased in both Th1 and Th17 cells when treated with CB839 (FIG. 22G).

TABLE 2
Metabolic pathways altered following CB839 treatment

| | Total | Hits | Raw p | Hits Discovered |
|---|---|---|---|---|
| Th1 Pathway | | | | |
| Alanine, aspartate and | 24 | 8 | 5.34E−08 | Fumaric acid; Pyruvic acid; Ureidosuccinic acid; L-Aspartic Acid; Arginosuccinic |
| glutamate metabolism | | | | acid; L-glutamic acid; L-glutamine; Oxoglutaric acid |
| Citrate cycle (TCA cycle) | 20 | 5 | 0.0001074 | Oxoglutaric acid; L-malic acid; Pyruvic Acid; Fumaric acid; Phosphoenolpyruvate |
| D-Glutamine and | 5 | 3 | 0.00015834 | L-Glutamic acid; L-glutamine; oxoglutaric acid |
| Dglutamate metabolism | | | | |
| Pyrimidine metabolism | 41 | 6 | 0.00048197 | L-Glutamine; 4,5-Dihydroorotic acid; Dihydrouracil; Cytidine monophosphate; |
| | | | | Cytidine; Ureidosuccinic acid |
| Arginine and proline | 44 | 6 | 0.00071444 | L-Glutamine; L-Aspartic acid; Argininosuccinic acid; L-Glutamic acid; L-4- |
| metabolism | | | | Hydroxyglutamate semialdehyde; Fumaric acid |
| Histidine metabolism | 15 | 3 | 0.0060105 | L-Glutamic acid; Methylimidazoleacetic acid; L-Aspartic acid |
| Butanoate metabolism | 22 | 3 | 0.017935 | Oxoglutaric acid; Pyruvic acid; 2-Hydroxyglutarate |
| Pyruvate metabolism | 23 | 3 | 0.020258 | Phosphoenolpyruvic acid; Pyruvic acid; L-Malic acid |
| Nitrogen metabolism | 9 | 2 | 0.021285 | L-Glutamic acid; L-Glutamine |
| Cysteine and methionine | 27 | 3 | 0.031146 | 5′-Methylthioadenosine; 2-Aminoacrylic acid; Pyruvic acid |
| metabolism | | | | |
| Th17 Pathway | | | | |
| Pentose phosphate pathway | 19 | 5 | 0.00433 | Deoxyribose 5-phosphate; D-Ribulose 5-phosphate; Sedoheptulose 7-phosphate; |
| | | | | 6-Phosphogluconic acid; D-Erythrose 4-phosphate |
| Alanine, aspartate and | 24 | 5 | 0.01245 | Argininosuccinic acid; L-Alanine; Ureidosuccinic acid; Succinic acid; |
| glutamate metabolism | | | | Glucosamine 6-phosphate |
| Phenylalanine, tyrosine and | 4 | 2 | 0.02017 | L-Phenylalanine; L-Tyrosine |
| tryptophan biosynthesis | | | | |
| Purine metabolism | 68 | 8 | 0.04882 | Xanthine; D-Ribulose 5-phosphate; ADP; Deoxyinosine; Hypoxanthine; |
| | | | | Guanosine triphosphate; Guanosine; Adenosine diphosphate ribose |

### GLS-Deficiency Has Little Effect on Resting T Cells But Modulates Activation

A GLS^{fl/fl} model was generated and crossed to CD4-Cre to specifically delete GLS late in T cell thymic development to test the role of GLS in T cells. Although GLS^{fl/fl} CD4-Cre T cells efficiently deleted Gls compared to control GLS^{fl/fl} T cells (FIG. 23A), lymphocyte frequencies and numbers were unaltered (FIG. 23B). Treg cells have been previously shown to be increased by ASCT2 or GOT1 deficiency (Nakaya et al., 2014; Xu et al., 2017b), but were unchanged with GLS-deficiency. Resting GLS^{fl/fl} CD4-Cre+CD4 T cells also had normal cell size and phenotype (FIG. 23C).

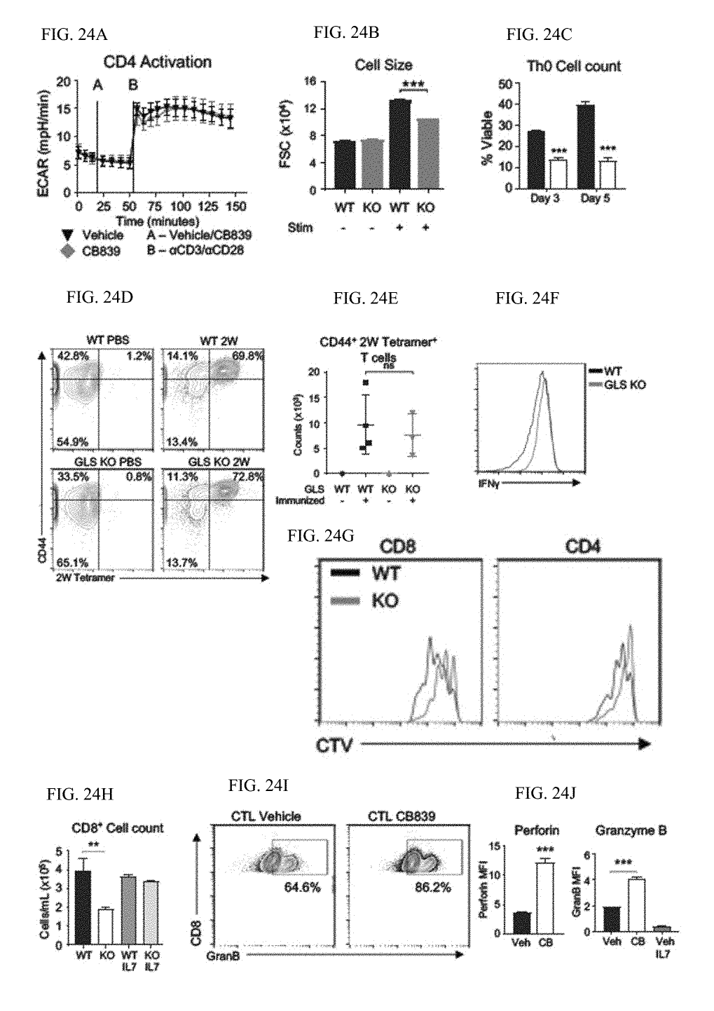

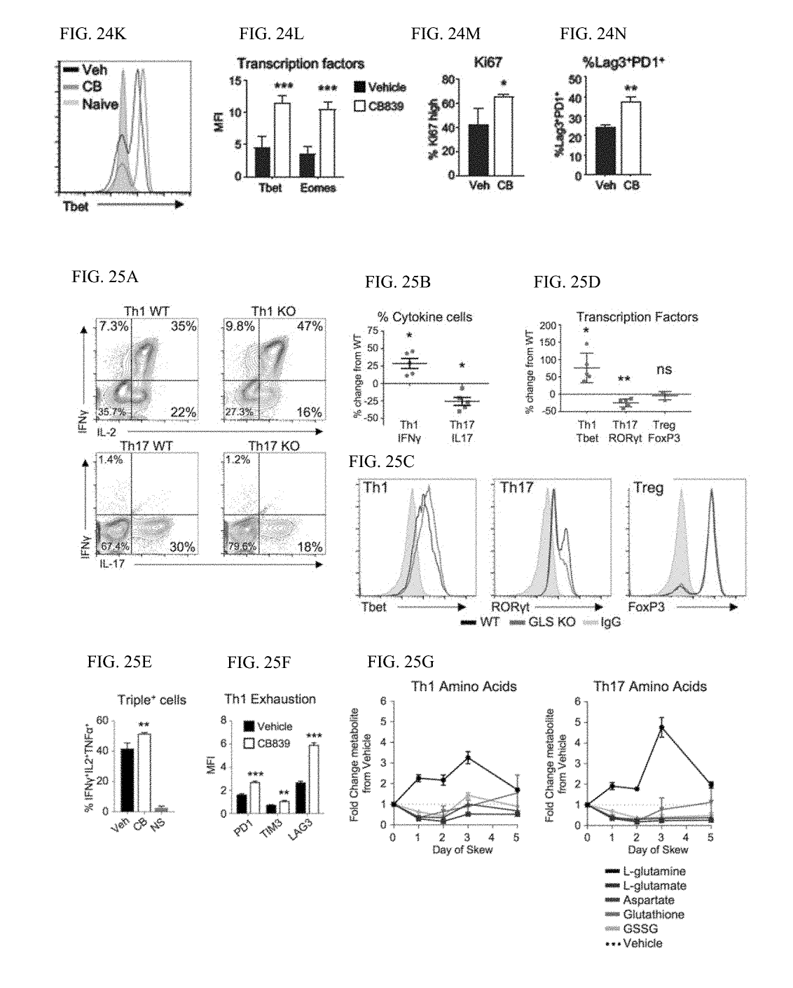

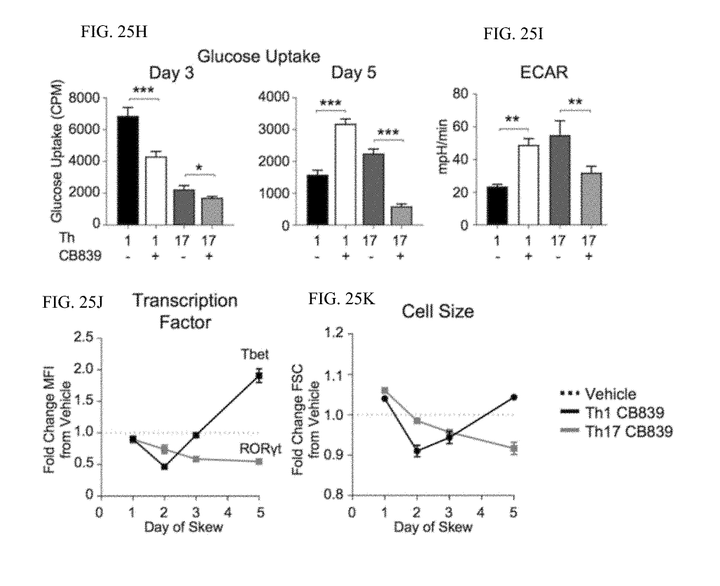

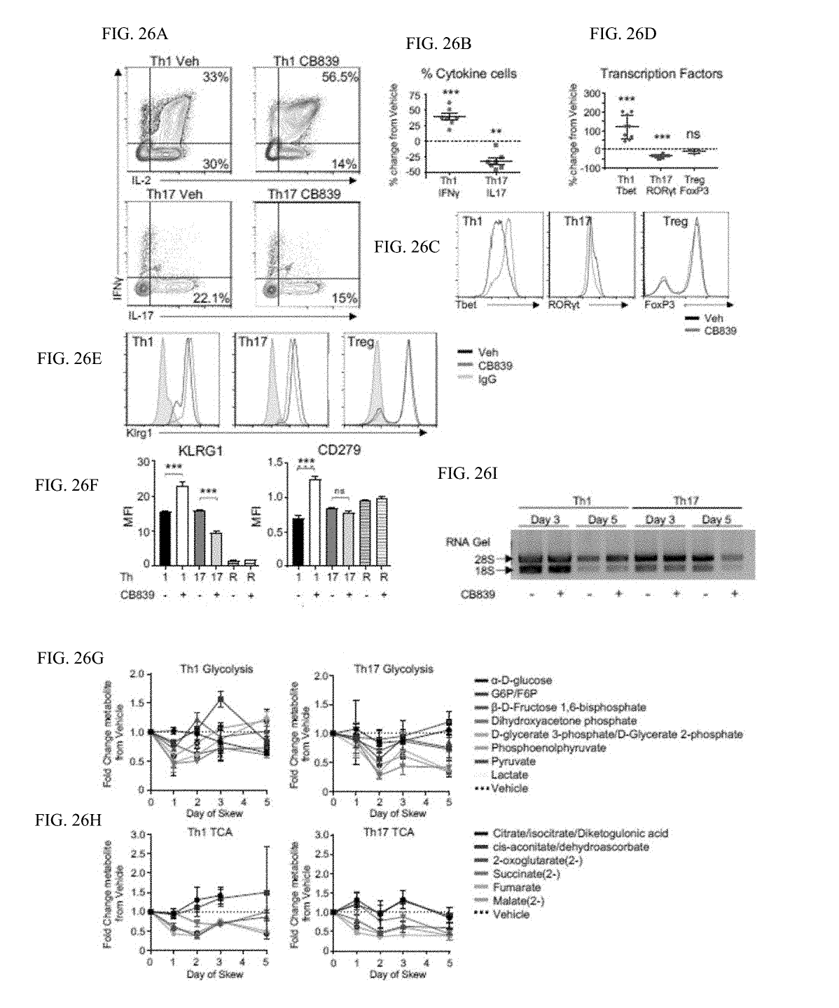

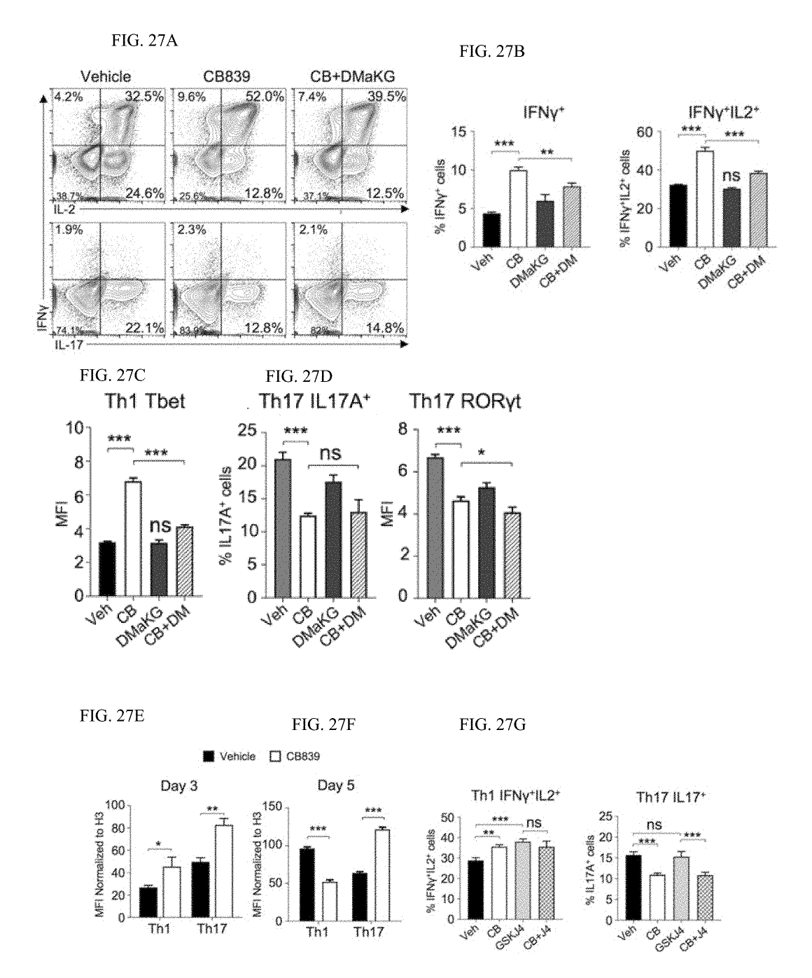

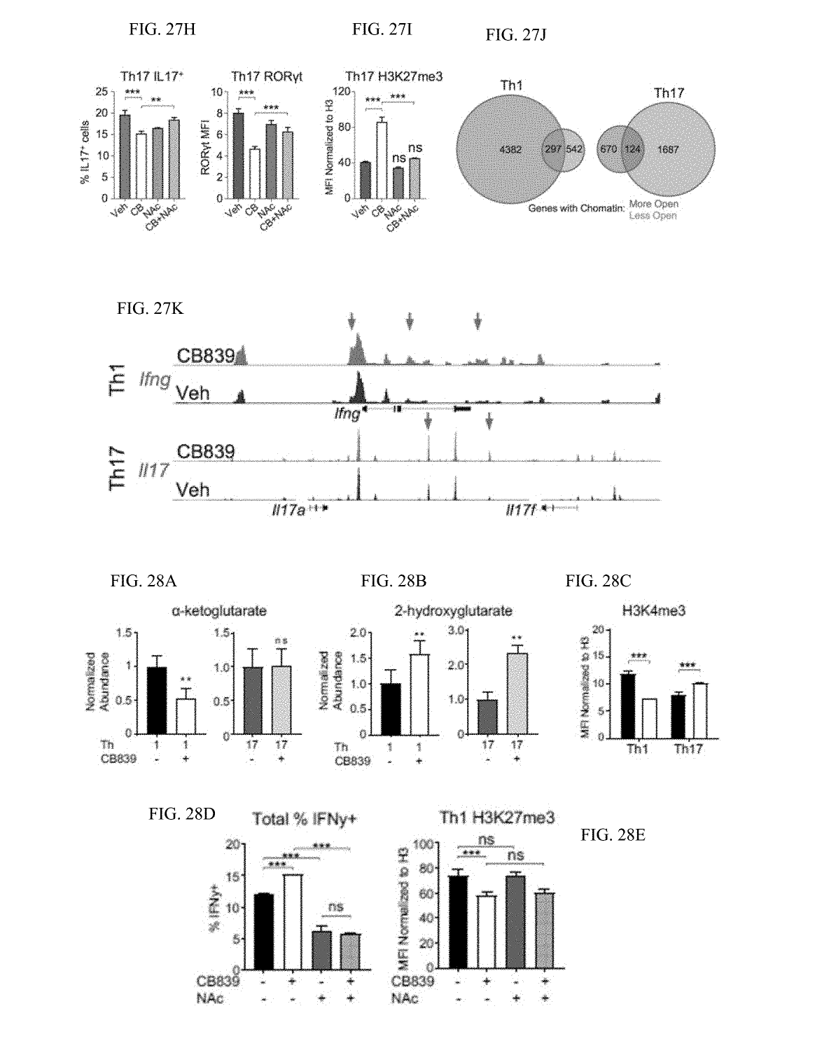

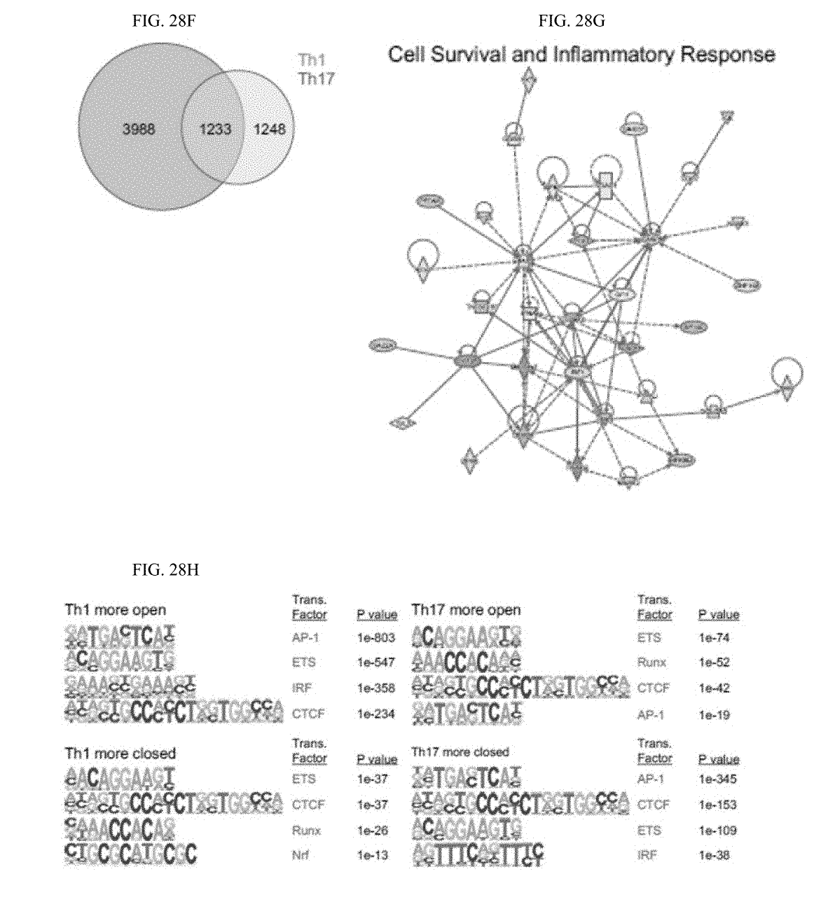

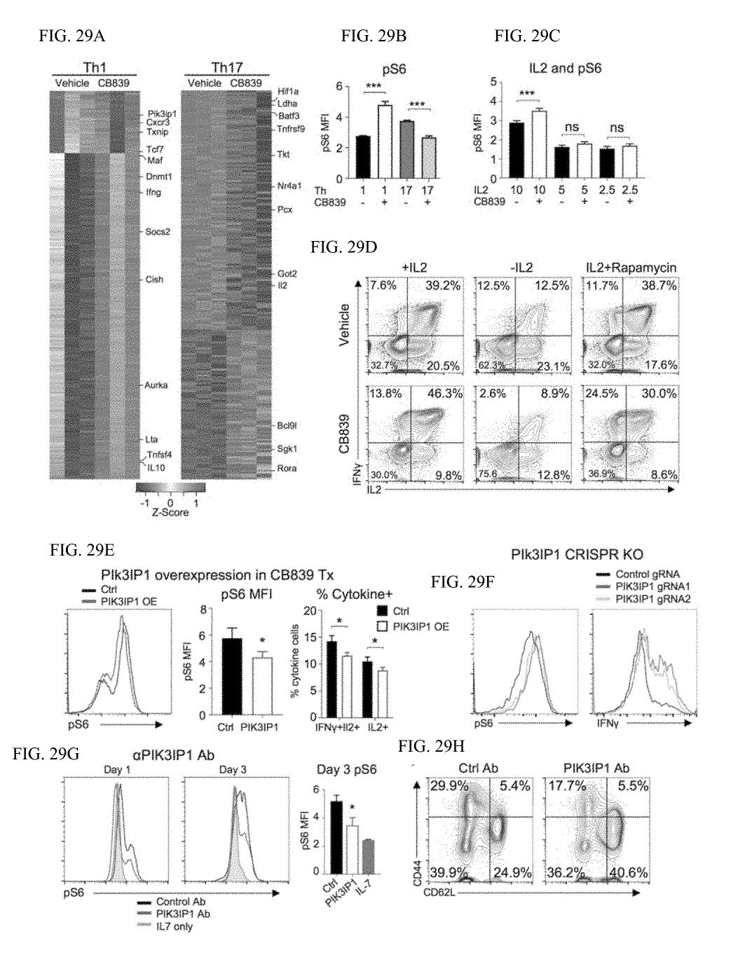

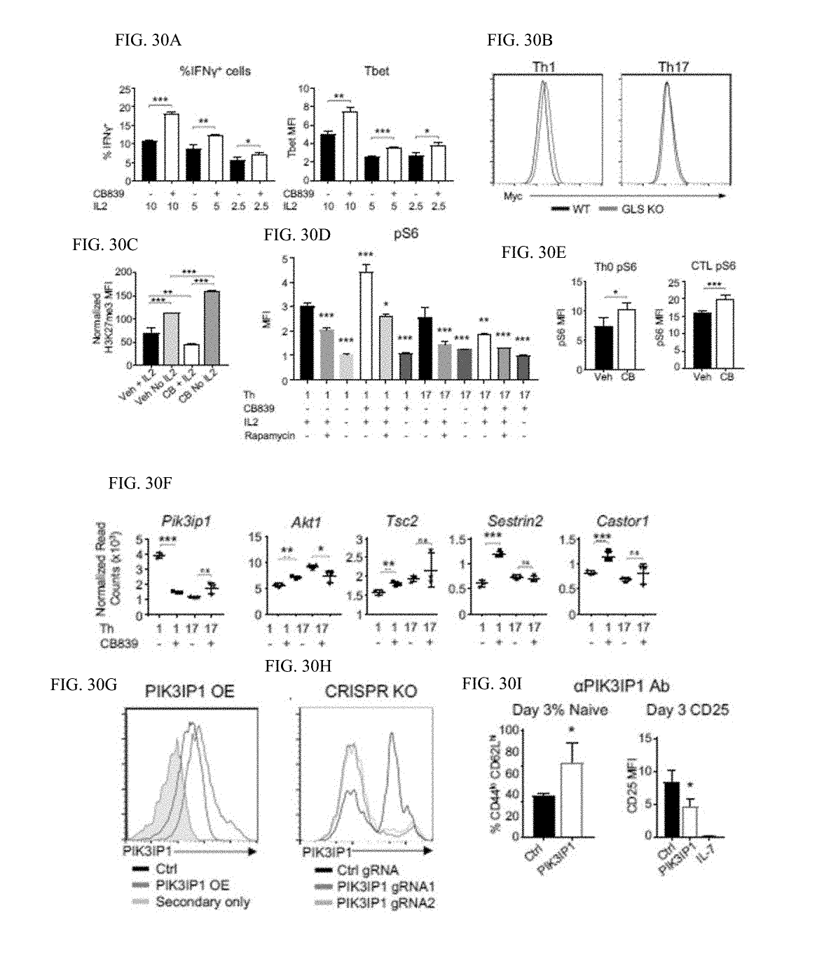

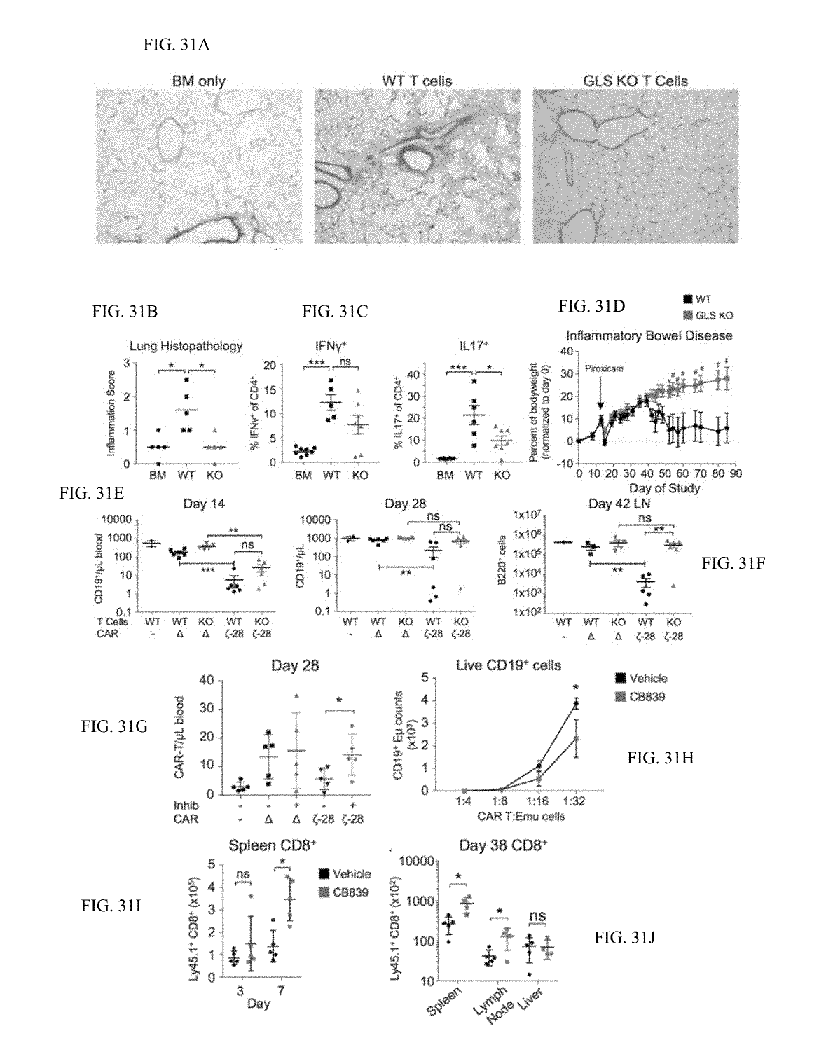

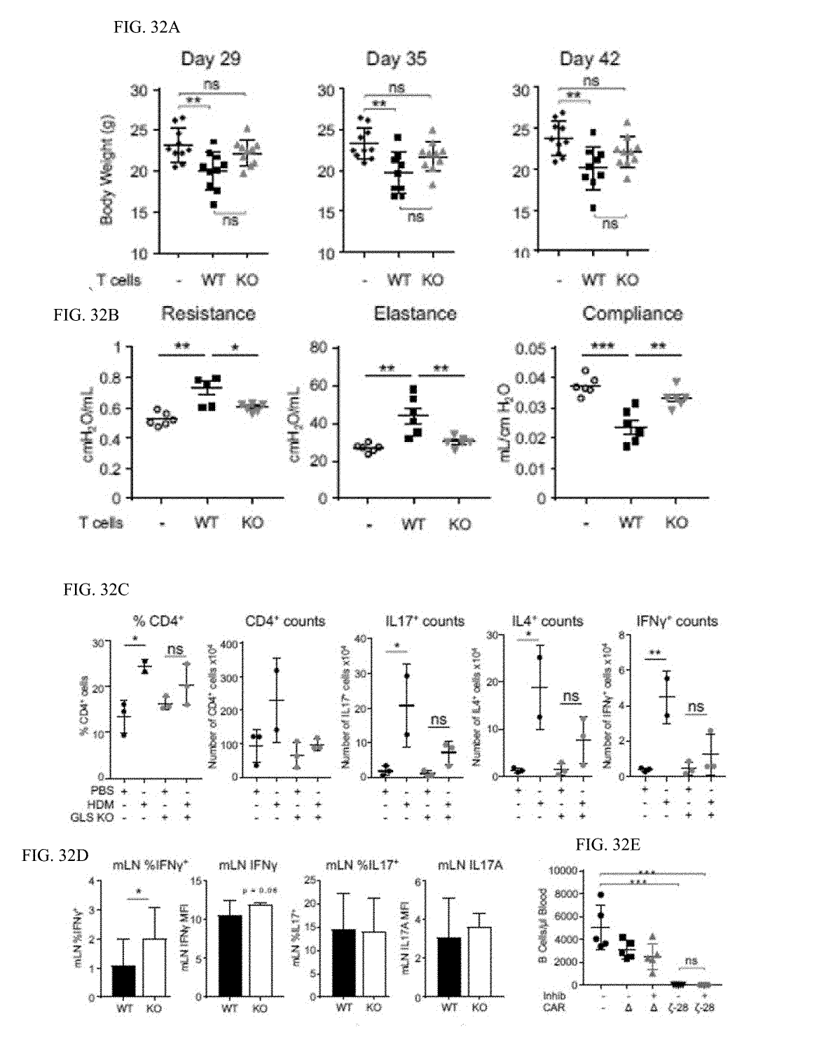

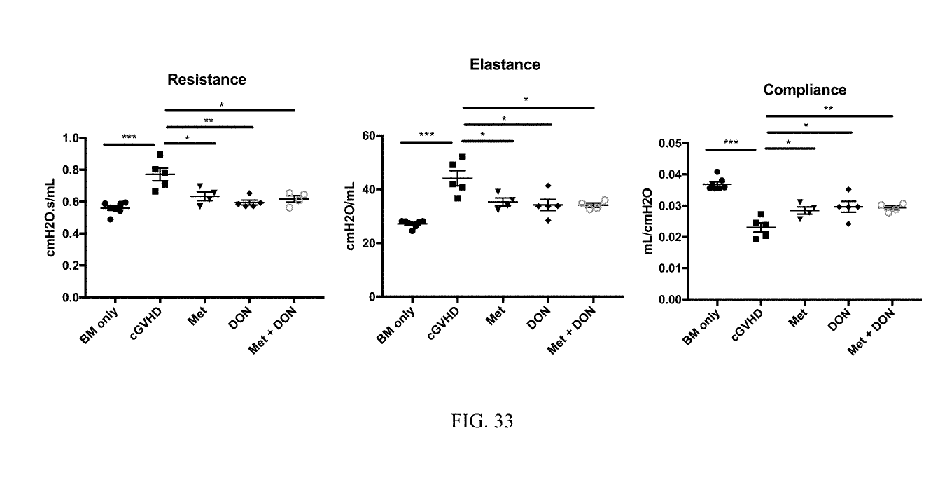

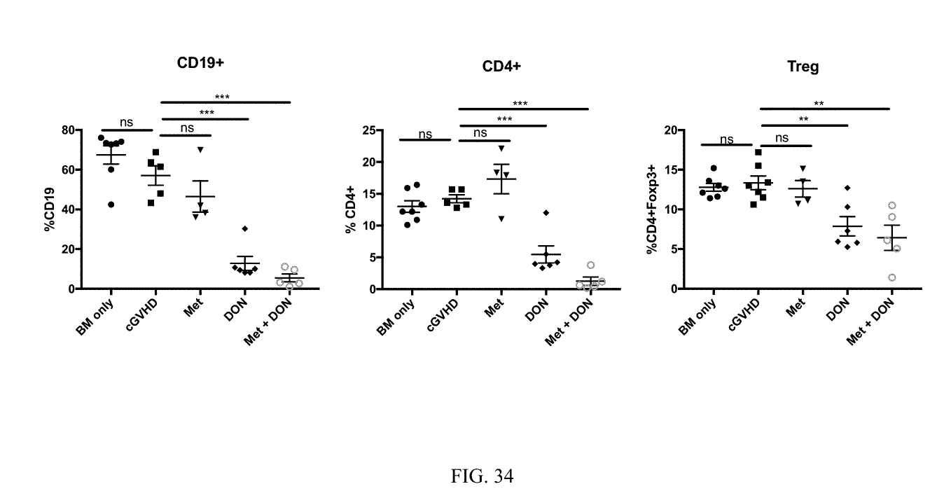

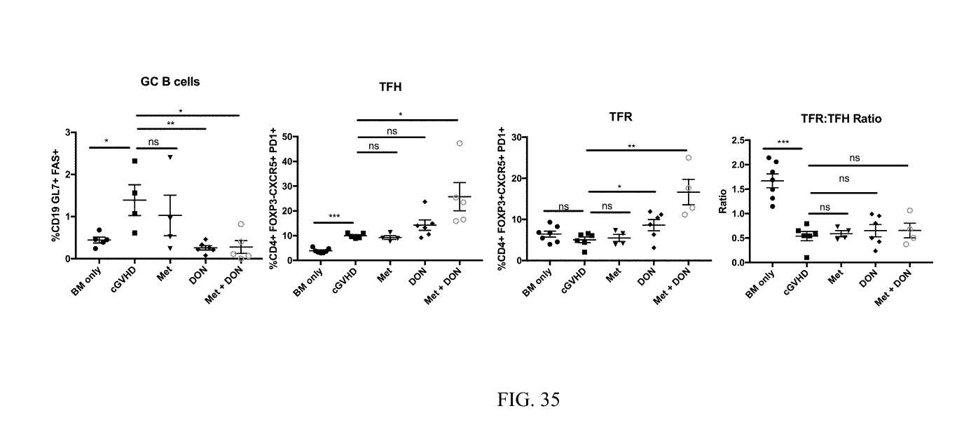

GLS-deficiency did, however, impact T cell activation. Measurement of immediate lactate secretion showed that acute GLS inhibition did not impair immediate events in T cell activation to rapidly induce glycolysis (FIG. 24A). However, in vitro stimulated GLS-deficient T cells failed to efficiently undergo blastogenesis and increase in cell size in the first two days (FIG. 24B). GLS-deficient CD4 T cells had reduced induction of CD25 and CD44, and downregulation of CD62L (FIG. 23D) at 48 hours. In addition, in vitro accumulation of viable stimulated T cells was reduced by GLS-deficiency (FIG. 24C). By day five of stimulation in IL2 (Th0 conditions), however, GLS-deficient CD4 T cells had adapted and activation markers were similar to control (FIG. 23E).

Delayed activation marker expression and proliferation of GLS-deficient T cells suggested impaired function and differentiation. Surprisingly, a greater frequency of GLS^{fl/fl} CD4-Cre+T cells produced IFNγ after five days in Th0 conditions than did control T cells (FIG. 23F, G). In addition, GLS-deficient cells that expressed IFNγ did so to a higher level than IFNγ-producing control T cells. IFNγ expression is regulated in part by the transcription factor, Tbet, and Tbet levels were elevated in activated GLS^{fl/fl} CD4-Cre+Th0 T cells (FIG. 23H). Similarly, inhibition of GLS with CB839 also led to greater expression of IFNγ and Tbet (FIGS. 23I-K).

The ability of T cells to adapt to GLS-deficiency and display enhanced function in vitro suggested in vivo responses may be altered. Control and GLS^{fl/fl} CD4-Cre mice were immunized, therefore, with 2W peptide to measure proliferation and IFNγ secretion. At eight days after immunization, 2W-MHC tetramer positive CD4 T cells proliferated similarly regardless of GLS expression (FIG. 24D, E). At day fifteen, IFNγ levels, however, were increased in GLS-deficient 2W-MHC tetramer positive T cells (FIG. 24F). In contrast, proliferation to weaker homeostatic cues was reduced for GLS^{fl/fl} CD4-Cre T cells in both spleen and lymph node compared to wild type T cells five days after transfer to recipient RAG1^{-/-} mice (FIG. 24G).

The dependence of CD8 T cells on GLS was assessed. Similar to CD4 cells, in vitro stimulated GLS^{fl/fl} CD4-Cre+CD8 T cells survived and accumulated less efficiently than control T cells (FIG. 24H). GLS^{fl/fl} CD4-Cre+CD8 T cells had increased expression of the effector protein Granzyme B (FIG. 23L) and Tbet (FIG. 23M). Acute inhibition of GLS with CB839 led to increased Granzyme B and Perforin after five days stimulation (FIG. 241, J). In addition to increased effector proteins, CB839-treated CD8 T cells expressed increased levels of Tbet and Eomes and markers of proliferation (FIG. 24K-M). However,

GLS-inhibition also increased the portion of CD8 T cells that expressed the inhibitory receptors Lag3 and PD-1 (FIG. 24N). GLS-deficiency thus can impair initial activation and proliferation of CD4 and CD8 cells, while promoting Th1-like and CTL effector programs that may ultimately sensitize to inhibition.

### GLS Plays Differential Roles in CD4 T Cell Effector Subsets

Given the differences in glutamine metabolism between Th1 and Th17 cells and spontaneous Th1 -like differentiation with IL2 in Th0 conditions, if GLS-deficiency differentially affected T cell subset specification and function was tested. Control and GLS^{fl/fl} CD4-Cre+or CB839-treated CD4 T cells were differentiated in vitro into Th1 and Th17 subsets. Similar to Th0 cells, a greater percentage of GLS^{fl/fl} CD4-Cre+T cells expressed IFNγ when in Th1 skewing conditions (FIG. 25A, B). Conversely, a decreased percentage of GLS-deficient T cells expressed IL17A when in Th17 skewing conditions. Expression of effector molecules and differentiation in Th1, Th17, and Treg are regulated by Tbet, RORγt, and FoxP3, respectively, and GLS-deficient T cells showed increased Tbet under Th1 conditions and decreased RORγt under Th17 conditions (FIG. 25C, D). In contrast, FoxP3 expression was unchanged in the absence of GLS. Similar results were obtained when GLS was acutely inhibited using CB839, as Th1, Th17, and Treg cytokine production and differentiation were increased, decreased, or unchanged, respectively (FIG. 26A-D).

GLS-deficiency promoted Th1 and suppressed Th17 differentiation and may affect plasticity and terminal fates. However, GLS-deficient T cells stimulated in Th17 conditions that failed to express RORγt and IL17 did not significantly elevate IFNγ or FoxP3 (FIGS. 21E, 25A, FIG. 26A). In contrast, GLS-deficient T cells stimulated in Th1 conditions showed evidence of excessive effector differentiation as the proportion of multi-functional Th1 cells (FIG. 25E) as well as expression of KLRG1 and inhibitory receptors, PD-1, Tim3, and Lag3 were elevated (FIG. 25F and FIG. 26E, F).

It was next assessed how GLS inhibition affected Th1 and Th17 metabolism and differentiation over time. Steady state levels of glutamine rapidly increased while glutamate and aspartate rapidly decreased in both Th1 and Th17 cells upon GLS inhibition (FIG. 25G). While levels of these metabolites partially recovered in GLS inhibitor-treated Th1 cells starting on day three, they remained low in treated Th17 cells. Likewise, oxidized glutathione (GSSG) recovered in Th1 but remained low in Th17. Similar trends of initial decrease followed by recovery in Th1 cells were observed in glycolytic and TCA cycle intermediates (FIG. 26G, H). Consistent with impaired early metabolism, flux measurements showed glucose uptake was reduced in both Th1 and Th17 cells on day three (FIG. 25H). By day five, however, Th1 cells had increased levels of glucose uptake and glycolytic flux relative to controls while Th17 remained impaired by GLS inhibition (FIGS. 25H, I).

Changes in metabolism occurred rapidly upon GLS inhibition and preceded Th1 and Th17 differentiation. Indeed, GLS inhibition led both Th1 and Th17 to have reduced levels of subset transcription factors and prevented an increase in cell size relative to control cells on days one and two after activation (FIG. 25J, K). By day five, however, Th1 cells had recovered and increased both cell size and Tbet expression. These data are consistent with overall changes in biomass, as total rRNA levels per cell were similar in GLS inhibitor or control treated T cells on day three, but Th1 had increased and Th17 had decreased rRNA levels by day five of GLS inhibition (FIG. 26I).

### GLS Affects Gene Expression and Chromatin Accessibility

Deficient GLS activity may alter differentiation through production of cofactors, including α-KG and 2-hydroxyglutarate (2-HG), for epigenetic marks and changes in chromatin status. Based on intracellular metabolite analysis by mass spectrometry, α-KG was reduced in CB839-treated Th1, but not Th17 cells, while 2-HG increased in both Th1 and Th17 (FIGS. 28A, B). The reduced α-KG in CB839-treated Th1 cells suggested that α-KG may become limiting to regulate Th1 differentiation and function. A cell-permeable α-KG analog, dimethyl 2-ketoglutarate (DMaKG), was tested to determine if provision of α-KG could restore normal Th1 specification of CB839-treated T cells (FIG. 27A-C). DMaKG did not reduce cytokine production in Th1 cells by itself. However, DMaKG rectified IFNγ production and Tbet expression of CB839-treated Th1 cells to control levels. In contrast, Th17 cells were not rescued by DMaKG and IL17 production and RORγt were unchanged or further decreased (FIG. 27A, D), suggesting a distinct mechanism of regulation for Th17 cells by GLS.

Histone tri-methylation was globally assessed by flow cytometry. Initially, GLS inhibition led to increased H3K27 tri-methylation (FIG. 27E). At later time points when Th1 differentiation was enhanced, however, CB839-treated Th1 and Th17 cells were found to have decreased or increased global H3K27 trimethylation, respectively (FIG. 27F). H3K4 trimethylation was similarly reduced or increased in Th1 and Th17 cells, respectively, at day five (FIG. 28C). Consistent with altered regulation of demethylation as a cause of Th1 differentiation upon GLS inhibition, treatment of T cells with an inhibitor of the histone demethylase JMJD3 also led to increased cytokine production in Th1 but not Th17 cells at day five (FIG. 27G).

The dependence of Th17 cells on GLS was not rescued by DMaKG, but Th17 cells can be highly sensitive to increased ROS (Gerriets et al., 2015). The glutathione mimic N-acetyl cysteine (NAC) was tested to rescue GLS-deficient Th17 cells. NAC treatment alone modestly reduced Th17 expression of IL17 and RORγt (FIG. 27H) while decreasing IFNγ secretion by Th1 (FIG. 28D). Th17 production of IL17 and expression of RORγt were partially restored to control levels when combined with CB839. The combination did not, however, increase Th1 production of IFNγ. Changes in Th17 inhibition by CB839 may be mediated through chromatin modifications as NAC also restored H3K27 trimethylation in GLS-deficient Th17 cells to control levels (FIG. 271) yet had no effect on H3K27 trimethylation in Th1 cells (FIG. 28E).

Because multiple epigenetic marks may be altered, the Assay for Transposase-Accessible Chromatin sequencing (ATACseq) was performed to determine if GLS deficiency altered chromatin accessibility after five days of Th1 and Th17 differentiation. CB839-treated Th1 cells had more genes with regions of increased accessibility than genes with decreased accessibility (FIG. 27J). Th17 cells however, had more genes with regions of reduced accessibility. While partially overlapping, affected genes were largely distinct for Th1 and Th17 cells (FIG. 28F). Key Th1 and Th17 genes showed changes, including the Ifng and Il17a/f loci in Th1 and Th17 cells, respectively (FIG. 27K). Further, Ingenuity Pathway analyses of genes with altered promoter accessibility in Th1 cells showed changes in networks of cell survival and inflammation (FIG. 27G). Analysis of promoter regions with altered accessibility identified recognition motifs for canonical T cell differentiation transcription factors, including AP-1, ETS, and IRF (FIG. 27H). These altered promoter regions were also enriched in CTCF recognition motifs.

Because altered chromatin accessibility can influence gene expression and T cell differentiation, T cells were cultured in Th1 or Th17 conditions with vehicle or CB839 and examined by RNA sequencing. Of the 200 genes with the most significantly altered expression in CB839-treated Th1 cells, the majority showed increased expression (FIG. 29A). Conversely, more of these genes were downregulated in Th17 cells. Functional annotation using gene set enrichment analyses showed that GLS-inhibition led to upregulation of specific pathways including those related to cell cycle, mTORC1, Myc, and IL2 signaling (Table 3). Similar gene sets were downregulated in Th17 cells treated with CB839.

TABLE 3
Gene Set Enrichment Analysis of Th1 and Th17 Cells Treated with GLS-Inhibitor

| | | | | | | |
|---|---|---|---|---|---|---|
| | # Genes | | # Genes | | | |
| | in Gene | | in Over- | | | FDR |
| Gene Set Name | Set (K) | Description | lap (k) | k/K | p-value | q-value |

Th1 Cells: Increased Pathways with CB839 Treatment

| | | | | | | |
|---|---|---|---|---|---|---|
| HALLMARK_E2F_TARGETS | 200 | Genes encoding cell cycle related targets | 135 | 0.675 | 7.54E−170 | 3.77E−168 |
| | | of E2F transcription factors | | | | |
| HALLMARK_G2M_CHECKPOINT | 200 | Genes involved in the G2/M checkpoint, as | 101 | 0.505 | 3.74E−108 | 9.36E−107 |
| | | in progression through the cell division | | | | |
| | | cycle | | | | |
| HALLMARK_MITOTIC_SPINDLE | 200 | Genes important for mitotic spindle assembly | 56 | 0.28 | 4.54E−43 | 7.57E−42 |
| HALLMARK_MTORC1_SIG- | 200 | Genes up-regulated through activation of | 47 | 0.235 | 1.53E−32 | 1.92E−31 |
| NALING | | mTORC1 complex | | | | |
| HALLMARK_MYC_TARGETS_V1 | 200 | A subgroup of genes regulated by MYC - | 43 | 0.215 | 3.55E−28 | 3.55E−27 |
| | | version 1 (v1) | | | | |
| HALLMARK_IL2_STAT5_SIG- | 200 | Genes up-regulated by STAT5 in response to | 37 | 0.185 | 5.01E−22 | 4.18E−21 |
| NALING | | IL2 stimulation | | | | |
| HALLMARK_GLYCOLYSIS | 200 | Genes encoding proteins involved in | 34 | 0.17 | 3.81E−19 | 2.72E−18 |
| | | glycolysis and gluconeogenesis | | | | |
| HALLMARK_DNA_REPAIR | 150 | Genes involved in DNA repair | 28 | 0.1867 | 4.05E−17 | 2.53E−16 |
| HALLMARK_ESTRO- | 200 | Genes defining late response to estrogen | 30 | 0.15 | 1.60E−15 | 8.01E−15 |
| GEN_RESPONSE_LATE | | | | | | |
| HALLMARK_P53_PATHWAY | 200 | Genes involved in p53 pathways and networks | 30 | 0.15 | 1.60E−15 | 8.01E−15 |

Th1 Cells: Decreased Pathways with CB839 Treatment

| | | | | | | |
|---|---|---|---|---|---|---|
| HALLMARK_KRAS_SIG- | 200 | Genes up-regulated by KRAS activation | 20 | 0.1 | 6.52E−10 | 3.26E−08 |
| NALING_UP | | | | | | |
| HALLMARK_COMPLEMENT | 200 | Genes encoding components of the complement | 18 | 0.09 | 2.43E−08 | 4.05E−07 |
| | | system, which is part of the innate immune | | | | |
| | | system | | | | |
| HALLMARK_INFLAM- | 200 | Genes defining inflammatory response | 18 | 0.09 | 2.43E−08 | 4.05E−07 |
| MATORY_RESPONSE | | | | | | |
| HALLMARK_ALLO- | 200 | Genes up-regulated during transplant rejection | 17 | 0.085 | 1.36E−07 | 1.70E−06 |
| GRAFT_REJECTION | | | | | | |
| HALLMARK_APICAL_JUNCTION | 200 | Genes encoding components of apical junction | 16 | 0.08 | 7.14E−07 | 7.14E−06 |
| | | complex | | | | |
| HALLMARK_APOPTOSIS | 161 | Genes mediating programmed cell death | 13 | 0.0807 | 6.99E−06 | 5.82E−05 |
| | | (apoptosis) by activation of caspases | | | | |
| HALLMARK_INTER- | 200 | Genes up-regulated in response to IFNG | 14 | 0.07 | 1.61E−05 | 1.15E−04 |
| FERON_GAMMA_RESPONSE | | [GeneID = 3458] | | | | |
| HALLMARK_ANGIOGENESIS | 36 | Genes up-regulated during formation of blood | 6 | 0.1667 | 3.86E−05 | 2.41E−04 |
| | | vessels (angiogenesis) | | | | |
| HALLMARK_UV_RESPONSE_DN | 144 | Genes down-regulated in response to ultraviolet | 11 | 0.0764 | 5.83E−05 | 2.65E−04 |
| | | (UV) radiation | | | | |
| HALLMARK_EPITHELIAL_MESEN- | 200 | Genes defining epithelial-mesenchymal | 13 | 0.065 | 6.88E−05 | 2.65E−04 |
| CHYMAL_TRANSITION | | transition, as in wound healing, fibrosis | | | | |
| | | and metastasis | | | | |
| HALLMARK_ESTRO- | 200 | Genes defining early response to estrogen | 13 | 0.065 | 6.88E−05 | 2.65E−04 |
| GEN_RESPONSE_EARLY | | | | | | |

Th17 Cells: Increased Pathways with CB839 Treatment

| | | | | | | |
|---|---|---|---|---|---|---|
| HALLMARK_INTER- | 200 | Genes up-regulated in response to IFNG | 25 | 0.125 | 2.94E−13 | 1.47E−11 |
| FERON_GAMMA_RESPONSE | | [GeneID = 3458] | | | | |
| HALLMARK_APICAL_JUNCTION | 200 | Genes encoding components of apical junction | 19 | 0.095 | 2.09E−08 | 3.48E−07 |
| | | complex | | | | |
| HALLMARK_P53_PATHWAY | 200 | Genes involved in p53 pathways and networks | 19 | 0.095 | 2.09E−08 | 3.48E−07 |
| HALLMARK_MYOGENESIS | 200 | Genes involved in development of skeletal | 18 | 0.09 | 1.12E−07 | 1.40E−06 |
| | | muscle (myogenesis) | | | | |
| HALLMARK_COMPLEMENT | 200 | Genes encoding components of the complement | 17 | 0.085 | 5.62E−07 | 4.01E−06 |
| | | system, which is part of the innate immune | | | | |
| | | system | | | | |
| HALLMARK_TNFA_SIG- | 200 | Genes regulated by NF-kB in response to TNF | 17 | 0.085 | 5.62E−07 | 4.01E−06 |
| NALING_VIA_NFKB | | [GeneID = 7124] | | | | |
| HALLMARK_XENO- | 200 | Genes encoding proteins involved in processing | 17 | 0.085 | 5.62E−07 | 4.01E−06 |
| BIOTIC_METABOLISM | | of drugs and other xenobiotics | | | | |
| HALLMARK_EPITHELIAL_MESEN- | 200 | Genes defining epithelial-mesenchymal | 16 | 0.08 | 2.66E−06 | 1.48E−05 |
| CHYMAL_TRANSITION | | transition, as in wound healing, fibrosis | | | | |
| | | and metastasis | | | | |
| HALLMARK_INFLAM- | 200 | Genes defining inflammatory response | 16 | 0.08 | 2.66E−06 | 1.48E−05 |
| MATORY_RESPONSE | | | | | | |
| HALLMARK_HYPOXIA | 200 | Genes up-regulated in response to low oxygen | 15 | 0.075 | 1.18E−05 | 5.90E−05 |
| | | levels (hypoxia) | | | | |

Th17 Cells: Decreased Pathways with CB839 Treatment

| | | | | | | |
|---|---|---|---|---|---|---|
| HALLMARK_E2F_TARGETS | 200 | Genes encoding cell cycle related targets of | 107 | 0.535 | 6.77E−130 | 3.38E−128 |
| | | E2F transcription factors | | | | |
| HALLMARK_G2M_CHECKPOINT | 200 | Genes involved in the G2/M checkpoint, as | 94 | 0.47 | 7.15E−107 | 1.79E−105 |
| | | in progression through the cell division | | | | |
| | | cycle | | | | |
| HALLMARK_MYC_TARGETS_V1 | 200 | A subgroup of genes regulated by MYC - | 75 | 0.375 | 4.76E−76 | 7.94E−75 |
| | | version 1 (v1) | | | | |
| HALLMARK_MTORC1_SIG- | 200 | Genes up-regulated through activation of | 70 | 0.35 | 1.64E−68 | 2.05E−67 |
| NALING | | mTORC1 complex | | | | |
| HALLMARK_MYC_TARGETS_V2 | 58 | A subgroup of genes regulated by MYC - | 31 | 0.5345 | 3.73E−38 | 3.73E−37 |
| | | version 2 (v2) | | | | |
| HALLMARK_IL2_STAT5_SIG- | 200 | Genes up-regulated by STAT5 in response to | 39 | 0.195 | 7.99E−28 | 6.66E−27 |
| NALING | | IL2 stimulation | | | | |
| HALLMARK_CHOLES- | 74 | Genes involved in cholesterol homeostasis | 25 | 0.3378 | 8.83E−25 | 6.31E−24 |
| TEROL_HOMEOSTASIS | | | | | | |
| HALLMARK_UNFOLD- | 113 | Genes up-regulated during unfolded protein | 29 | 0.2566 | 1.17E−24 | 7.29E−24 |
| ED_PROTEIN_RESPONSE | | response, a cellular stress response related | | | | |
| | | to the endoplasmic reticulum | | | | |
| HALLMARK_TNFA_SIG- | 200 | Genes regulated by NF-kB in response to TNF | 34 | 0.17 | 2.14E−22 | 1.19E−21 |
| NALING_VIA_NFKB | | [GeneID = 7124] | | | | |
| HALLMARK_GLYCOLYSIS | 200 | Genes encoding proteins involved in glycolysis | 31 | 0.155 | 2.53E−19 | 1.26E−18 |
| | | and gluconeogenesis | | | | |

IL2 signaling activates mTORC1 to promote Myc signaling, glycolysis, and Th1 effector differentiation. Given enrichment in these pathways by RNAseq, the contribution of IL2/mTORC1 signaling was tested to increased effector function of GLS-deficient Th1 cells. Levels of the mTORC1 downstream target phospho-S6 were measured in Th1 and Th17 cells differentiated in IL2 and the presence or absence of CB839. GLS-inhibition led to increased phospho-S6 in Th1 and decreased phospho-S6 in Th17 cells (FIG. 29B). IL2 played a key role to promote phospho-S6, as increased phospho-S6, IFNγ, and Tbet in CB839-treated Th1 were dependent on IL2 (FIG. 29C, FIG. 30A). Consistent with mTOR regulation of Myc protein, GLS-inhibition modestly increased Myc in Th1 but not Th17 cells (FIG. 30B). While GLS-inhibition in the presence of IL2 led to enhanced differentiation and a hypomethylated state, T cells hypermethylated H3K27 upon treatment with CB839 in the absence of IL2 (FIG. 30C). The role of mTORC1 signaling in GLS-mediated regulation of Th1 cells was directly tested by treatment of cells on day three after activation with rapamycin. While rapamycin treatment at this time had no effect on control Th1 cells, it reduced phospho-S6 and cytokine production in CB839-treated Th1 cells (FIG. 29D, FIG. 30D). A similar mechanism may occur for regulation of Th0 and CTL, as GLS-inhibition also led to enhanced phospho-S6 for these cells in the presence of IL2 (FIG. 30E).

Several regulators of mTORC1 signaling were altered by GLS-inhibition in Th1 cells by RNA-Seq, including Pik3ip1, Akt, Tsc2, Sestrin2, and Castor1 (FIG. 30F). Of these, Pik3ip1 was most strongly downregulated in Th1 cells by GLS inhibition. Restoring PIK3IP1 in CB839-treated Th1 cells by retroviral transduction was sufficient to reduce phospho-S6, cytokine secretion, and Tbet expression (FIG. 29E, FIG. 30G). Conversely, CRISPR genetic deletion of Pik3ip1 in primary T cells led to increased phospho-S6 and IFNγ production (FIG. 29F, FIG. 30H). PIK3IP1 is a transmembrane protein and treatment of stimulated T cells with anti-PIK3IP1 antibody directed against the extracellular domain suppressed phospho-S6 (FIG. 29G) and T cell activation as evidenced by downregulation of CD25, CD44, and CD62L (FIG. 29H, FIG. 30I). Together, these data suggest that PIK3IP1 levels can contribute to mTORC1 activity and effector function in Th1 cells while Th17 cells are dependent on GLS-mediated regulation of cellular redox state.

### GLS Regulates In Vivo For Inflammatory Effector T Cell Responses

It was next tested if Th17 cells require GLS to elicit inflammation in vivo. Allogenic bone marrow was transplanted alone or with control and GLSfl/flCD4-Cre+ T cells to induce a model of IL17-dependent chronic Graft-vs-Host Disease (cGvHD). Recipient mice were weighed regularly and GLS-deficient allogenic T cells led to less weight loss than control T cells (32A). cGvHD is a multi-organ disease (Panoskaltsis-Mortari et al., 2007) and mouse models of cGvHD include lung inflammation. Histological examination showed that GLS-deficient T cells reduced lung immune infiltrate and clinical inflammation score (FIG. 31A, B) and caused significantly less airway functional impairment than control T cells (FIG. 32B). Immunologically, GLS deficiency reduced IL17 producing CD4 cells, with a trend towards reduced IFNγ (FIG. 31C). GLS was also critical in an independent model of Th17-mediated lung inflammation, in which control and GLS-deficient animals sensitized and challenged in the airway with House Dust Mite antigen and LPS failed to accumulate CD4 T cells and produce inflammatory cytokine in the lung (FIG. 32C). Inflammatory bowel disease (IBD) also involves Th17 cells and we found that while adoptive transfer of control T cells led to weight loss and inflammation, mice that received GLS-deficient T cells maintained weight (FIG. 31D). Despite partial protection from disease, a greater percentage of GLS-deficient T cells in the mesenteric lymph nodes produced IFNγ, consistent with a preferential Th1 response (FIG. 32D).

The role of GLS-deficiency to enhance Th1 and CTL function was next tested in vivo. Control and GLS^{fl/fl} CD4-Cre T cells were evaluated in a murine Chimeric Antigen Receptor (CAR) model for the ability to eliminate endogenous target B cells and persist in vivo. T cells were in vitro transduced with CAR-T expression vectors either lacking a cytoplasmic tail (Δ) or with a CD3ζ-CD28 (28-ζ) intracellular tail and adoptively transferred into animals conditioned with cyclophosphamide. Fourteen days after T cell transfer, endogenous CD19-expressing B cells were significantly reduced by both control and GLS^{fl/fl} CD4-Cre CAR-T cells (FIG. 31E). After 28 days, however, B cells had accumulated in recipients of GLSfl/flCD4-Cre CAR-T cells and were fully recovered in lymph nodes by day 42 (FIG. 31E, F). Consistent with upregulation of inhibitory receptors upon activation, GLS-deficient T cells appeared unable to sustain an effector response in the absence of GLS activity in vivo.

Because GLS-inhibition altered chromatin accessibility in Th1 cells in vitro, it was possible that transient treatment with CB839 could induce long lasting effect. T cells were treated with vehicle or CB839 during in vitro transduction to express CARs and tested for subsequent in vivo function. Vehicle and CB839-treated CAR T cells were equally capable of eliminating CD19+ targets in vivo (FIG. 32E). In vitro CB839-treated CAR-T cells accumulated in vivo to a greater extent than untreated CAR-T cells (FIG. 31G) and showed greater ability to eliminate B cell leukemia cells in vitro (FIG. 31H). This increased ability of Th1 and CD8 effector T cells to proliferate or persist following transient GLS inhibition was not specific to CAR T cells. CD8 T cells bearing a Pmel-specific TCR transgene treated with CB839 in vitro prior to adoptive transfer also accumulated to greater numbers in vivo by day 7 when challenged with an antigen-expressing vaccinia virus (FIG. 31I) and increased cell numbers persisted for greater than 5 weeks (FIG. 31J). Thus, chronic or complete GLS deficiency impairs T cell responses in vivo, while transient in vitro inhibition may enhance subsequent Th1 and CD8 CTL effector function and long-lasting cell numbers in vivo.

### Materials and Methods

### Mice

Mice were obtained from the Jackson laboratory or described previously. GLS^{fl/fl} animals were obtained as Glstmla(KOMP)Mbp embryonic stem cells (Project ID: CSD29307) from the KOMP that were blastocyst microinjected to generate mice (Duke University Transgenic and Knockout Shared Resource) and crossed to FLP transgenic animals. Progeny were then crossed with CD4-CRE transgenic mice to develop the GLS^{fl/fl} CD4-CRE (GLS KO). In all cases comparing wild type to GLS KO, sex-matched and age-matched littermates were used (8 to 14 weeks of age unless otherwise stated). Animals were genotyped for floxed alleles and CRE allele. All procedures were performed under IACUC-approved protocols.

### T Cell In Vitro Activation and Skew Experiments

T cells were cultured in RPMI 1640 supplemented with glutamine, HEPES, BME, and Pen/Strep unless otherwise noted. CB839 was dosed at 1 μM (activation) or 500 nM (differentiation), GSKJ4 (Selleckchem, Cat #: S7070) at 1 μM, dimethyl-2-oxoglutarate (DMaKG) (Sigma Aldrich, Cat #: 349631) at 1.5 mM. and rapamycin (Sigma, Cat #: 553210) at 5 nM. Briefly, naïve CD4 T cells were isolated from wild type animals (WT) and GLS1fl/fl CD4-CRE+ mice (GLS KO) and activated over various time points via 5 ug/mL anti-CD³/ₐnti-CD28 antibodies plate bound (ThermoFisher, CD3: Cat #16-0031-85, CD28: Cat #16-0281-85). Non-stimulated CD4 samples were maintained using 10 ng/mL IL-7 (Peprotech, Cat #: 217-17). For skewing experiments, naïve CD4 T cells from WT or KO animals were plated with subset-specific cytokines and stimulated with feeder layer of irradiated splenocytes. Th0 experiments were run in skewing condition (+αCD3 antibody) without additional cytokines. After 3 days, cells were split with fresh media and stimulated with or without 10 ng/mL IL-2 (Cat #: 14-8021-64) for a further 2 days. For intracellular cytokine stains, cells were re-stimulated using PMA/ionomycin in the presence of GolgiPlug (Cat #: 555029) for 4 hours, then fixed and stained for intracellular subset-specific cytokines using fix/perm kit (Cat #: 554714). For all other intracellular or intranuclear stains such as transcription factor, pS6, C-MYC, H3K4me3, H3K27me3, and total H3 protein, cells were removed from media, stained for surface markers, fixed, then stained for intracellular proteins using fix/perm kit (Cat #00-5223-56, 00-5123-43). Cell proliferation was assessed by staining naïve CD4+ cells with Cell Trace Violet proliferative dye at 5 μM (Cat #: c34557).

### Homeostatic Proliferation

Homeostatic proliferation was measured as previously described (Jacobs et al., 2010). Briefly, naïve CD4+ and CD8+ T cells were isolated from GLSfl/flCD4-Cre and wild-type Thy1.1+ mice. Cells were mixed in a 1:1 ratio and stained with proliferative dye CellTrace Violet (Cat #: c34557). Cells were transplanted by i.v. injection into recipient RAG knockout mice 8 weeks of age. Five days after injection, spleen and mesenteric lymph node were collected, homogenized, and stained with antibodies against CD4, CD8, and Thy1.1 for flow cytometry analysis.

### ATAC-Sequencing Experiments

Crude nuclei pellets for ATAC-seq were isolated according to Buenrostro et. al (Buenrostro et al., 2013) with modifications. Briefly, naïve CD4 T cells were skewed to Th1 and Th17 subsets in vitro with vehicle or in the presence of 0.5 μM CB839. At Day 5, T cells were re-isolated for CD4+ cells using CD4+ negative selection kit (Cat #: 130-104-454). 1×105 cells were removed for nuclei extraction in ATAC-Seq lysing buffer. Cells were exposed to Tn5+ adaptor proteins from Nextera DNA for 30 min at 37° C. and immediately placed on ice. Transposed eluate was amplified via PCR using Nextera DNA preparation kit (Cat #: FC-121-1030), NEBNext High-fidelity 2× PCR mix (Cat #: M0541), and multiplexed (Cat #: FC-121-1011). Samples were purified using Zymo DNA cleanup kit (Cat #: D4011). QC of samples was run on bioanalyzer before being sent for sequencing.

### RNA Sequencing Experiments

Th1 and Th17 cells were skewed with or without CB839 over 5 days and total RNA extracted for RNAseq (Cat #: 74104). RNA was sent to VANderbilt Technologies for Advanced GEnomics (VANTAGE) core at Vanderbilt University. Libraries were prepared using 50 ng of total RNA using the NEBNext Ultra RNA Library Kit for Illumina (Cat #E7530) and sequenced on HiSeq3000 at 75 bp paired-end. Each sample was analyzed in triplicate. Sequencing reads were aligned against the Mouse GENCODE genome, Version M14 (Jan. 2017 freeze, GRCm38, Ensembl 89) using the Spliced Transcripts Alignment to a Reference (STAR) software (ref: 26187010 and 23104886). Reads were preprocessed and index using SAMtools (ref: 19505943). Mapped reads were assigned to gene features and quantified using featureCounts (ref: 24227677). Normalization and differential expression was performed using DESeq2 (Love et al., 2014). Skewed lymphocytes with and without CB839 were compared in both Th1 and Th17 groups. The top most significantly differentially expressed genes (FDR<0.01 and Log2 difference greater than 0.5 in magnitude) were considered for subsequent functional enrichment using Geneset Enrichment Analysis. The top 200 most differentially expressed genes were used for unsupervised hierarchical cluster analysis and visualized using heatmap representations.

### PCR

Pan T cells were isolated and purified using Miltenyi isolation kit (Cat #: 130-095-130). Genomic DNA was generated using Kapa express Extract kit (Cat #: KR0370). Primers targeted over exon 10 and exon 11 were generated for wild type band with a melting temperature of 54° C.: Forward: ACGAGAAAGTGGAGATCG (SEQ ID NO:17); Reverse: GCCTTCTGGAAAACA (SEQ ID NO:18). PCR product was then run on a 1% agarose gel with ethidium bromide and visualized by GelDoc XR (Cat #: 1708195).

### Glucose Uptake

Glucose uptake assays were performed as previously described (Macintyre et al.,2014). Naïve CD4+ T cells were differentiated into Th1 and Th17 cells, in triplicate, in the presence or absence of CB839 over five days and spun down after reisolation using CD4 kit as previously described. At day 3 and 5, cells were removed, washed twice in PBS, counted, then rested in 1 mL Kreb's Ringers HEPES (KRH) for at least 10 minutes. Cells were spun and resuspended to 5×105 cells/50 μL KRH for glucose uptake assay. Briefly, 3H-2-deoxyglucose was suspended in KRH bubble layered in oil, and cells were added to this bubble. Cells were incubated for 10 minutes at 37° C. Immediately after incubation, reaction was quenched with 200 μM phloretin (Calbiochem, Cat #: 524488). Cells were spun, washed, and then resuspended in scintilation fluid for counting on Beckman-Coulter scintillation counter (3H, 1 min/sample read).

### Extracellular Flux Analyses (Seahorse)

Experiments were carried out on Agilent Seahorse XF96 bioanalyzer (Agilent). Briefly, wild type CD4+ cells were isolated as previous and activated for 3 days on αCD3/CD28 coated plates as previously described, or skewed to Th1 and Th17 subsets as described above. T cells were isolated and spun onto XF96 Cell-Tak (BD Bioscience, Cat #: 354240) coated plates and rested in Seahorse XF RPMI 1640 media supplemented with glutamine, sodium pyruvate, and glucose. For immediate metabolic response, 1 μM CB839 and 5 μM UK5099 (Cat #: PZ0160-5MG) were injected separately or in combination, and OCR and ECAR measured. For activation response, 1 uM CB839 was injected into IL-7 maintained naïve CD4+ T cells in seahorse medium and allowed to incubate for 20 minutes, followed by soluble αCD3/CD28 injection.

### Mass Spectrometry

¹³C Tracing. To measure 13C-Glucose tracing in T cell activation, CD4 cells were stimulated on 5 μg/mL anti-CD3/CD28 for 3 days. At day 3, cells were pooled, washed 3× in PBS, and re-stimulated in presence of 1 uM CB839 or Vehicle (DMSO) and 11 mM 13C glucose (Cambridge Isotope Labs, Cat #: CLM-1396-1). Cells were incubated for 24 hours at 37 oC, then scraped and combined in triplicate. Cells were rinsed with 0.9% saline and metabolites were extracted in methanol. Metabolites measured by LC High-Resolution Mass Spectrometer (LC-HRMS) using a Q-Exactive machine as previously described(Liberti et al., 2017). The time-dependent glucose labeling pattern was modeled as with the following equation:

$$\frac{[ {X}^{*} ]}{{X}^{T}} = 1 - {e}^{- \frac{{f}_{X}}{{X}^{T}} t}$$

In which [X*] is the concentration of labeled glucose, is the total concentration (both labeled and unlabeled) of glucose, is the glucose production flux. This model was fit to glucose MIDs using the fit( ) function in MATLAB to determine relative glucose production fluxes. Relative glucose pool sizes were estimated from MS signal intensities.

Differentiation. CD4 cells were isolated as previously described and differentiated in subset-specific medium in the presence of vehicle or CB839 (in triplicate) for 3 days, split at day 3 with new media and IL-2, then allowed to incubate a further 2 days. At day 5, wells were combined, cells washed 1× in MACS buffer and re-isolated for CD4 via AutoMACS Pro automated magnetic separator (Miltenyi, Cat #: 130-092-545). Metabolites from Th1 and Th17 cells were extracted and analyzed by LC-HRMS using a Q-Exactive as described previously (Gerriets et al., 2015). Data were range scaled and analyzed using Metaboanalyst 3.5 (Xia and Wishart, 2002) to generate heat maps and for principle component analyses.

### Immunoblotting

Immunoblots were performed as previously described (Jacobs et al., 2008) with the following modifications. Cells lysed with RIPA buffer and Halt protease/phosphatase cocktail inhibitors (Life Tech, Cat #: 78443). Protein was quantified by Pierce BCA kit II (Cat #: 23227). Actin blots were visualized by near infrared fluorescence via Licorr Odyssey imager. GLS blots were visualized by chemiluminescence using anti-rabbit conjugated horseradish peroxidase. The antibodies used for westerns were: GLS (Cat #: GTX81012, 1:1000), β-Actin (Cat#: 8226, 1:10,000).

Viral Infection with PIK3IP1

Naïve CD4+ T cells were isolated from wild type C57BL6 mice. T cells were stimulated in Th1 and Th17 skewing conditions plus vehicle of CB839 as previously described. These were incubated for 16 hours with a feeder layer of irradiated splenocytes. Plasmid constructs MSCV-PIK3IP-IRES-Thy1.1 (“PIK3IP1”) and control vector MSCVIRES-Thy1.1 (“Control”) were used to transfect Plat-E cells. T cells were then infected with cell supernatant containing retrovirus and polybrene and rested for 48 hours. Cells were split at Day 3 in new media containing IL-2 (10 ng/mL) and then incubated for 48 hours before removing for intracellular cytokine and transcription factor staining by flow cytometry as described above.

### CRISPR/CAS9 PIK3IP1

Naïve CD4+ T cells were isolated from Cas9 transgenic mice (The Jackson Laboratory, Stock #024858) aged 10-12 weeks old. T cells were plated on an αCD3/CD28 coated 24-well plate and one day after activation, cells were transduced with viral supernatant prepared from PLAT-E cells (Cat #: RV-101) transfected with a solution of 2000 μg DNA (empty vector pMx-U6-empty-GFP or two different PIK3IP1 targeting guide RNA containing vectors pMx-U6-PIK3IP1-GFP). T cells with the viral particles were centrifuged at 2000 rpm for 2 hours at 37° C., followed by incubation for 2 hours at 37° C. and 5% CO2. The media was then replaced with 1 mL fresh Th1 skewing media and incubated overnight. This was repeated a second time on day 2 of T cell activation. Cells were collected ten days post activation for pS6, intracellular cytokine production, and transcription factor staining by flow cytometry as described.

### PIK3IP1 Antibody In Vitro

Naïve CD4+ T cells were isolated from C57BL6 mice and activated on αCD3/CD28-coated 24 well plates at 1×106 cells/well with either control antibody (Cat #bs-0295P) or PIK3IP1 antibody (Cat #16826-1-AP) at 0.5 μg/mL. Cells were incubated at 37° C. for 72 hours and cells removed at 24, 48, and 72 hours for flow cytometry analysis of activation.

### In Vivo Graft Versus Host Disease

Induction of Graft vs Host Disease (cGVHD) was performed as previously described (Panoskaltsis-Mortari et al., 2007). Briefly, mice were lethally irradiated the day before bone marrow transplant. Mice were dosed with cyclophosphamide (Cytoxan, Bristol Myers Squibb, Seattle Wash.) at 120 mg/kg/day on days −3 and −2. Recipient irradiated mice were transplanted via caudal vein with 15×106 T-cell depleted allogeneic marrow with 1×106 cells splenic CD4+ cells from WT or GLS KO mice, or control (no CD4+ T cells). Mice were assessed for lung elasticity, resistance, and compliance at Day 28 by whole body plethysmography using the Flexivent system (Scireq, Montreal, PQ, Canada). Histological assessment of GVHD was assessed as previously described (Blazar et al., 1998).

### Asthma Model

Female mice were administered intranasal sensitization of either PBS alone or a combination of 100 μg house dust mite extract (Greer, Lenoir, N.C.) and 0.1 ug LPS from Escherichia coli 0111:B4 (Sigma, St. Louis, Mo.) in 50 ul of PBS. Sensitizations were performed on Day 0, 7, and 14. Mice were harvested 24 hours post-challenge, and lung homogenates were digested to single cells and analyzed for cytokine production and transcription factors by flow cytometry.

### In Vivo Vaccinia Viral Response

Spleens from pmel-1 Ly5.1 (B6.Cg-Thy-1a/Cy Tg [TcraTcrb] 8Rest/J) mice were used to generate a single cell suspension and treated with ACK buffer to lyse red blood cells. Splenocytes were stimulated in vitro with 1 μM human glycoprotein 100 nine-mer peptide (hgp10025-33) and expanded in culture medium containing IL-2 for 7 days along with 1 μM CB839 or DMSO vehicle. Subsequently, one million CD8+ cells from each condition were transferred by IV injection into recipient Ly5.2 C57BL/6 mice. Immediately following transfer, mice were infected with rhgp100 vaccinina virus (1×10⁷ plaque-forming units (PFU)). At the indicated time points following transfer, recipient mouse blood or tissues were collected for analysis.

### Immunization With 2W Peptide

10-14 week old GLS WT and KO animals were injected with 10 μg 2 W peptide (Genscript, Peptide EAWGALANWAVDSA) emulsified with Complete Freunds Adjuvant or PBS control and injected subcutaneously in the rear flank as previously described (Moon et al., 2007) and rested for 8 days. At day 8, inguinal lymph nodes and spleens were removed and isolated. MHCII-specific CD4 cells were isolated and purified with APC-conjugated tetramers (generously provided by Dr. Marc Jenkins laboratory, Minneapolis, Minn.) using Miltenyi LS magnetic columns (Cat #: 130-042-401) and stained for extracellular and intracellular targets. Intracellular IFNγ was measured in a separate experiment on day 15 after immunization.

In vitro CAR T Cell Co-Culture With Target Eμ B ALL Cells

T cells were isolated from wild type C57BL6 spleens using the Pan T Cell isolation kit (Cat #: 130-095-130) and were activated on anti-CD3 anti-CD28 coated plates with IL2 for four days with or without CB839. On days 1 and 2, T cells were transduced with retrovirus produced by Plat-E cells carrying the CAR construct targeting CD19 with GFP reporter. On day 4, CAR T cells were washed three times to remove any drug remnants and plated to equal concentrations on a 96 well plate at 5×105 cells per well and serial dilutions thereof. 5×105 Emu cells, a CD19+ B cell acute lymphoblastic leukemia cell line

(Generously provided by Dr. Davila Lab) were then added to every well to assay cell numbers. CD19+ and GFP+events were stained and counted by flow for each well after 72 hours.

### In Vivo CAR T Cells

CAR T cells were produced as previously described (Li et al., 2017). Briefly, spleen T cells were isolated from wild-type B6, Thy1.1, or GLS KO mice at day 0. Cells were then activated with mouse CD3/CD28 Dynabeads and 30 IU/mL recombinant human IL2. At day 1 and 2, cells were spin transduced twice with retrovirus carrying CARs. At day 3, cells were fed with fresh medium. At day 4, transduced T cells were harvested, beads removed, evaluated for viability, transduction efficiency, immune phenotype and ready for use. For CB839 treated CAR T cells, compound was added to the culture at day 1, 2 and 3. For in vivo study, C57B6 mice (n=25) were i.p. injected with cyclophosphamide (CTX) at 300 mg/kg. Mice were i.v. injected with 3×105 CAR T cells one day after CTX injection. Peripheral blood (PB) samples were collected after CAR T injection, stained with B cell and T cell antibodies and subjected to flow cytometry. CountBright beads were added to measure B and T cell numbers.

### Colitis/IBD Induction

Colitis was induced by adoptive transfer of 0.4×10⁶ purified (>99% purity) CD4+ CD25-CD45RB^{hi} cells i.p. in 200 ul of PBS. Spleen and lymph node suspensions were used first to purify CD4+ cells using magnetic bead cell separation with a StemCell Kit and these cells were stained with anti-CD4, anti-CD25 and anti-CD45RB for further flow sorting using a FACS Diva flow cytometer (Becton-Dickinson) with purities over 95% of the indicated populations. Mice that received adoptive transfers of different cell genotypes were always cohoused in the same cages to avoid differences due to microbiota composition divergence during colitis development. Mice were treated with the NSAID Piroxicam to induce gut damage and initiate disease and animals were weighed over time. Mice that reached humane endpoints and were euthanized were maintained in the analysis at the final weight. At the end of the experiment, mesenteric lymph nodes were isolated and single cell suspensions were analyzed for cytokine production.

### Statistical Analysis

Statistical analyses were performed with Prism software version 7.01 (GraphPad Software, La Jolla Calif., USA, www.graphpad.com) using the student T-test, oneway ANOVA, or one-sample T-test. Longitudinal data was analyzed by two-way ANOVA followed by Tukey's test and followed up with one-way ANOVA or T-test at one specific time point as specified. Statistically significant results are indicated (*p<0.05, **p<0.01, ***p<0.001) and ns indicates select non-significant data. Error bars show mean±Standard Deviation unless otherwise indicated. RNA-Seq data were analyzed by DESeq2 (Love et al., 2014) in R (Team, 2017).

### Example 4

### Treating and/or Preventing Graft Versus Host Disease

### Materials and Methods

### Mice

Mice were obtained from the Jackson laboratory or as described elsewhere (Young et al., 2011 PLoS One 6(8):e23205). GLS^{fl/fl} animals were obtained as embryonic stem cells from the KOMP and crossed to FLP transgenic animals to delete the Neo cassette. These progeny were then crossed with CD4-CRE transgenic mice to develop the GLS^{fl/fl} CD4-CRE (GLS KO). In all cases comparing wild type to GLS KO, sex-matched and age-matched littermates were used. All procedures were performed under appropriate IACUC-approved protocols.

### In Vivo Graft Versus Host Disease

Induction of Graft vs Host Disease (cGVHD) was performed as described elsewhere (see, e.g., Panoskaltsis-Mortari et al., 2007 Am J Respir Crit Care Med. 176(7):713-723). Briefly, mice were lethally irradiated the day before bone marrow (BM) transplant. Mice were dosed with cyclophosphamide (Cytoxan, Bristol Myers Squibb, Seattle Wash.) at 120 mg/kg/day on days −3 and −2. Recipient irradiated mice were transplanted via caudal vein with 10×10⁶ T-cell depleted allogeneic marrow with 73.5×10³ purified splenic T cells from WT or GLS KO mice, or control (no CD4+ T cells). Mice were assessed for lung elasticity, resistance, and compliance at Day 28 by whole body plethysmography using the Flexivent system (Scireq, Montreal, PQ, Canada). Histological assessment of GVHD was assessed as described elsewhere (see, e.g., Blazar et al., 1998 Blood 92(10):3949-3959).

### Treatment Groups

6-Diazo-5-Oxo-L-Norleucine (DON) was administered to mice conditioned with cyclophosphamide (Cytoxan, Bristol Myers Squibb, Seattle Wash.) and total body irradiation.

TABLE 4
Treatment conditions.

| | | Conditioning | TBI | | TCD | Purified | |
|---|---|---|---|---|---|---|---|
| Group | N | D -3 and -2 | D -1 | Recipient | BM | Splenic T cells | Day 28-56 |
| 1 | 10 | 120 mg/kg | 830X | B10.BR | WT B6 | — | — |
| | | Cytoxan | | | 10 × 10⁶ | | |
| 2 | 10 | 120 mg/kg | 830X | B10.BR | WT B6 | WT B6 | — |
| | | Cytoxan | | | 10 × 10⁶ | 73.5 × 10³ | |
| 3 | 5 | 120 mg/kg | 830X | B10.BR | WT B6 | WT B6 | Metformin |
| | | Cytoxan | | | 10 × 10⁶ | 73.5 × 10³ | 150 mg/kg |
| | | | | | | | Daily, IP |
| 4 | 8 | 120 mg/kg | 830X | B10.BR | WT B6 | WT B6 | DON |
| | | Cytoxan | | | 10 × 10⁶ | 73.5 × 10³ | 1.6 mg/kg |
| | | | | | | | EOD, IP |
| 5 | 7 | 120 mg/kg | 830X | B10.BR | WT B6 | WT B6 | Metformin/DON |
| | | Cytoxan | | | 10 × 10⁶ | 73.5 × 10³ | Combination |

### Results

To determine if inhibiting GLS can improve cGVHD, DON (with or without metformin) was administered to mice that were transplanted with WT T cell depleted bone marrow with WT purified splenic T cells beginning on day 28 after transplant.

Administering DON, with or without metformin, to mice improved pulmonary function in the mice (FIG. 33).

Administering DON, with or without metformin, to mice reduced the percentage of lymphocytes in the mice (FIG. 34).

Administering DON, with or without metformin, to mice decreased GC B cell frequency, increased T_{FH} frequency, and improved T_{FR}:T_{FH} ratios in mice (FIG. 35).

Together FIG. 33, FIG. 34, and FIG. 35 show that DON can improve pulmonary functions, decrease GC B cell frequencies, and increase T_{FR} frequencies.

These results demonstrate that DON can be used treat and/or prevent GVHD.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or preventing graft-versus-host disease (GVHD) in a subject, said method comprising:
administering a therapeutically effective amount of a glutaminolysis inhibitor to the subject.

2. The method of claim 1, wherein the glutaminolysis inhibitor is 6-Diazo-5-Oxo-L-Norleucine (DON).

3. The method of claim 2, wherein the DON is administered to the subject at a dose of about 0.5 mg to about 50 mg of the DON per kilogram (kg) of the subject.

4. The method of claim 3, wherein the DON is administered to the subject at a dose of about 1.6 mg of the DON per kg of the subject.

5. The method of claim 1, wherein the glutaminolysis inhibitor is administered to the subject at least once a day.

6. The method of claim 1, wherein the glutaminolysis inhibitor is administered intraperitoneally.

7. The method of claim 1, wherein the subject has received a hematopoietic stem cell transplant.

8. The method of claim 7, wherein the hematopoietic stem cell transplant is an allogeneic hematopoietic stem-cell transplant.

9. The method of claim 7, wherein the hematopoietic stem cell transplant is a bone marrow transplant.

10. The method of claim 1, wherein the administering occurs prior to the subject receiving the hematopoietic stem cell transplant.

11. The method of claim 1, wherein the administering occurs coincidentally with the subject receiving the hematopoietic stem cell transplant.

12. The method of claim 1, wherein the administering occurs after the subject has received the hematopoietic stem cell transplant.

13. The method of claim 1, wherein GVHD is treated in the subject when the GVHD or one or more symptoms associated with the GVHD is reversed, alleviated or inhibited.

14. The method of claim 1, wherein GVHD is prevented in the subject when the GVHD or one or more symptoms associated with GVHD is avoided or precluded.

15. The method of claim 1, wherein the GVHD is chronic GVHD.

16. The method of claim 1, wherein the GVHD is acute GVHD.

17. A method of treating or preventing graft-versus-host disease (GVHD) in a subject, said method comprising:
contacting donor T cells with a therapeutically effective amount of a glutaminolysis inhibitor.

18. The method of claim 17, wherein the glutaminolysis inhibitor is 6-Diazo-5-Oxo-L-Norleucine (DON).

19. The method of claim 17, wherein the donor T cells are from hematopoietic stem cells.

20. The method of claim 17, wherein the donor T cells are contacted ex vivo.
